SEQUENCE LISTING (10 sequences)

SEQ ID NO: 1 - DNA, 95 bp, Homo sapiens
  source 1..95: mol_type = genomic DNA
tgaaggtttaattccgcatgttttagagctagaaatagcaagttaaaataaggctagtccgttatcaacttgaaaaagtggcaccgagtcggtgc

SEQ ID NO: 2 - DNA, 96 bp, Homo sapiens
  source 1..96: mol_type = genomic DNA
cgttgctgcctctttgggttgttttagagctagaaatagcaagttaaaataaggctagtccgttatcaacttgaaaaagtggcaccgagtcggtgc

SEQ ID NO: 3 - DNA, 22 bp, Homo sapiens
  source 1..22: mol_type = genomic DNA
tgaaggtttaattccgcatagg

SEQ ID NO: 4 - DNA, 23 bp, Homo sapiens
  source 1..23: mol_type = genomic DNA
cgttgctgcctctttgggtttgg

SEQ ID NO: 5 - RNA, 26 nt, Homo sapiens
  source 1..26: mol_type = genomic RNA
ttaattccgcataggttatttcdtdt

SEQ ID NO: 6 - RNA, 26 nt, Homo sapiens
  source 1..26: mol_type = genomic RNA
gaaataacctatgcggaattaadtdt

SEQ ID NO: 7 - RNA, 25 nt, Homo sapiens
  source 1..25: mol_type = genomic RNA
aagataagacagataattcccdtdt

SEQ ID NO: 8 - RNA, 25 nt, Homo sapiens
  source 1..25: mol_type = genomic RNA
gggaattatctgtcttatcttdtdt

SEQ ID NO: 9 - RNA, 22 nt, Homo sapiens
  source 1..22: mol_type = genomic RNA
gaaataacctatgcggaattaa

SEQ ID NO: 10 - RNA, 21 nt, Homo sapiens
  source 1..21: mol_type = genomic RNA
gggaattatctgtcttatctt

(19) United States
(12) Patent Application Publication
(10) Pub. No.: US 2024/0166998 A1
(43) Pub. Date: May 23, 2024

(54) METHOD FOR PRODUCING HEMATOPOIETIC CELLS FROM STEM CELLS USING VASCULAR ORGANOIDS

(71) Applicant: Artec Biotech, Inc., Chicago, IL (US)

(72) Inventors: Yekaterina Galat, Chicago, IL (US); Xenia A. Glukhova, Chicago, IL (US); Igor P. Beletsky, Chicago, IL (US); Vasiliy Galat, Chicago, IL (US)

(21) Appl. No.: 18/492,472

(22) Filed: Oct. 23, 2023

Related U.S. Application Data
(63) Continuation of application No. PCT/US2022/025992, filed on Apr. 22, 2022.
(60) Provisional application No. 63/256,142, filed on Oct. 15, 2021.
(60) Provisional application No. 63/178,284, filed on Apr. 22, 2021.

(51) Int. Cl. C12N 5/0783; A61K 35/17; A61P 35/02; C12N 9/22; C12N 15/113

(52) U.S. Cl. CPC C12N 5/0646; A61K 35/17; A61P 35/02; C12N 9/22; C12N 15/1138; C12N 2310/14; C12N 2310/20; C12N 2500/38; C12N 2500/84; C12N 2501/125; C12N 2501/2302; C12N 2501/2303; C12N 2501/2307; C12N 2501/2315; C12N 2501/26; C12N 2501/415; C12N 2506/02; C12N 2506/45; C12N 2510/00

(57) ABSTRACT

Disclosed herein are compositions and methods for a cell culture system for differentiating stem cells into, e.g., engraftable hematopoietic progenitor cells (HPCs), myeloid and/or lymphoid hematopoietic cells. In particular, the invention relates to producing hemogenic clusters of cells from pluripotent stem cells (e.g., embryonic stem cells (ESCs) or induced pluripotent stem cells (iPSCs)), culturing the clusters of cells to form a vascular organoid, and derivation of HPCs, natural killer (NK) cells, or myeloid cells using the vascular organoid. The present disclosure further relates to methods of modifying various stem cells and/or hematopoietic cells to, e.g., suppress the proliferation of tumor cells, eliminate senescent cells, modulate pathogen infection (e.g., bacterial infection or viral infection) or inhibit pathogen infection, and uses thereof. In certain aspects, stem cells and/or NK cells provided herein lack expression of NKG2A and/or function, or show reduced expression and/or function of NKG2A. In certain other aspects, stem cells and/or NK cells provided herein comprise modified NKG2A. Methods of using cells of the present disclosure, e.g., in the treatment of cancer and infectious disease are also provided.

### CROSS-REFERENCE TO RELATED APPLICATIONS

The present application claims priority to PCT application serial no. PCT/US2022/025992, filed on Apr. 22, 2022, which claims priority to U.S. provisional application Ser. No. 63/178,284, filed on Apr. 22, 2021 and U.S. provisional application Ser. No. 63/256,142, filed Oct. 15, 2021, each herein incorporated by reference in their entireties.

### SEQUENCE LISTING

The present application contains a Sequence Listing which is submitted electronically in XML format and is incorporated by reference in its entirety. Said XML file was created on Feb. 14, 2024, is named XML Sequence ABI-102-US-CON.xml and 9,980 bytes in size.

### FIELD OF THE INVENTION

Disclosed herein are compositions and methods for a cell culture system for differentiating stem cells into, e.g., engraftable hematopoietic progenitor cells (HPCs), myeloid and/or lymphoid hematopoietic cells. The present disclosure further relates to methods of modifying stem cells and/or hematopoietic cells. Methods of using such cells, e.g., in the treatment of cancer and infectious diseases are also provided.

### BACKGROUND

The generation of human hematopoietic progenitor cells from embryonic or induced pluripotent stem cells (iPSCs) would benefit myriad areas of research including hematopoiesis, cellular therapy, cancer immunotherapy, induced transplant tolerance, treatment for autoimmune diseases, and drug/toxicity testing. Substantial progress has been made in the development of effective approaches for hematopoietic differentiation from human pluripotent stem cells (hPSCs) [1-4]. In combination with gene correction systems, hematopoietic differentiation provides an avenue for disease modeling [5, 6] and cellular therapies [7, 8]. Transplantation of hematopoietic stem cells (HSCs), which originate via the definitive hematopoietic program, offers potential treatment for a variety of hematological disorders [9, 10]. A hallmark of definitive hematopoiesis is the capacity of hematopoietic progenitors to produce cells of lymphoid lineage [11]. To date, the generation of various types of lymphoid cells, such as T lymphocytes [12-15], natural killer (NK) cells [16], and induced NK (INK) cells [17], and limited lymphoid B-cell potential from hPSCs has been reported.

The development of a fully defined system for generation of functional hematopoietic cell types, especially lymphocytes, remains a significant challenge. The identification of molecular mechanisms and factors driving the hematopoietic specification of various blood lineages from hPSCs is critical in overcoming this limitation [19]. Originally, hematopoietic induction was established by co-culture with mouse stromal cells [20]. Currently, there are two main approaches to refining the conditions for induction of blood lineages. The first is based on selection of appropriate transcriptional regulators in gain-of-function experiments [21-23]. The second approach relies on the development of a cytokine regimen based on manipulations of the pathways of embryonic hematopoiesis [13, 24-26]. The signaling landscape is crucial for fate determination at the initial stages of differentiation. Presently, feeder-free defined systems for hematopoietic induction from pluripotent stem cells, in particular, include prolonged incubations with various cytokines that make the differentiation process complex and time consuming.

Evidence that immunotherapy targeting senescent cells combats aging and chronic diseases, and subsequently extends the healthy lifespan continues to mount [27]. Cellular anti-senescence therapy is an attractive approach as it avoids harmful side effects and toxicity associated with senolytics, small molecules, gene therapy and immunomodulators. NK cells play an instrumental role in immunosurveillance and senescent cell elimination, thus emerging as a front-line candidate to fight aging. NKs are also considered a first line of immune defense against cancer and viral infection. It has been hypothesized that SARS-COV2, similar to other viruses (e.g. influenza virus, IFV) can induce senescence to increase replication rate, thereby leading to massive presence of senescent cells in elderly and comorbid patients, which may exacerbate the mortality rate [28]. However, during aging and over the course of various cancers and infections, NKs undergo progressive functional exhaustion, rendering these cells unable to kill infected cells or cancer cells. Often, this is associated with the increased expression of the inhibitory receptor NKG2A, which binds to HLA-E that is frequently up-regulated on cancer and senescent cells [30]. It has recently been shown that blocking the interaction between HLA-E and NKG2A boosts immune responses of NKs against senescent cells [30]. Deriving NKs from iPSCs, in particular, would offer various advantages as therapeutic tools, since such cells may exhibit a “rejuvenated” phenotype as described for other type of cells [41], which is especially important for immunocompromised and/or aging patients with weakened immune systems. A major advantage of hPSC derived-NK technology is that it also allows for genetic modification(s) e.g., knockout, or knockdown genes associated with functional exhaustion, further enhancing the immune response and cell killing capabilities.

### SUMMARY OF THE INVENTION

As specified in the Background section, above, there is a need in the art for a more efficient, cost effective and controllable approach for hematopoietic differentiation of pluripotent stem cells to provide reliable source of patient-specific vascular progenitors, hematopoietic stem cells, myeloid and lymphoid cells for cellular therapies including lymphocyte immune-therapies, e.g., NK cell therapies, as well to support disease modeling and drug screening research efforts. There exists a need for NK cell therapies, in particular, that are not impeded by functional exhaustion that may be useful for combating cellular senescence, cancer, and/or infectious diseases.

Disclosed herein are compositions and methods for a cell culture system for differentiating stem cells into e.g., engraftable hematopoietic progenitor cells (HPCs), myeloid and/or lymphoid hematopoietic cells. The present disclosure further relates to methods of modifying stem cells and/or hematopoietic cells. Methods of using such cells, e.g., in the treatment of ischemia, stem cell transplant, neutropenia, cancer and infectious diseases are also provided.

In one aspect is provided a method for producing a population of hematopoietic progenitor cells from pluripotent stem cells comprising: a) plating the pluripotent stem cells at a seeding density of 1-5×10⁶ cells per 60 mm dish and culturing them overnight to produce colonies of 10-100 cells, b) inducing differentiation of the cells generated in step (a) by incubating said cells for about 2 days in an induction media comprising ascorbic acid and a Wnt activator, c) removing the Wnt activator and continuing incubation for about 4-7 days to produce a first population of hematopoietic progenitor cells, and d) optionally, purifying the first population of hematopoietic progenitor cells generated in step (c).

In certain aspects of the methods for producing a population of hematopoietic progenitor cells from pluripotent stem cells disclosed herein, the method may further comprise: e) detaching cell layers formed in step (c) enzymatically and/or mechanically, and plating them onto matrix-coated dishes or onto a monolayer of feeder cells, f) culturing the cells plated in step (e) for about 2-5 days in alpha-MEM differentiation media comprising 10% FBS, ascorbic acid, and one or more cytokines, resulting in the formation of a second population of hematopoietic progenitor cells, and g) optionally, purifying the second population of hematopoietic progenitor cells generated in step (f).

In certain aspects of the methods for producing a population of hematopoietic progenitor cells from pluripotent stem cells disclosed herein, the method may further comprise: e) detaching cell layers formed in step (c) enzymatically and/or mechanically, and plating them onto matrix-coated dishes or onto a monolayer of feeder cells, f) culturing the cells plated in step (e) for about 9-13 days in alpha-MEM differentiation media comprising 10% FBS, ascorbic acid, and one or more cytokines, resulting in the formation of a third population of hematopoietic progenitor cells from a three-dimensional vascular organoid, and g) optionally, purifying the third population of hematopoietic progenitor cells generated in step (f).

In various embodiments of any of the above-described methods, the purifying step may be achieved by isolating CD34+ cells. In some embodiments, step (a) may further comprises dissociating the pluripotent cells into single cells using a Ca²⁺ and Mg²⁺ free phosphate-buffered saline (PBS) solution before the plating step. In some embodiments, the pluripotent stem cells may be plated onto a plate that is coated with fibronectin. In some embodiments, the one or more cytokines used in step (f) comprise SCF, IL-3, and TPO.

In another aspect is provided a method for producing natural killer (NK) cells from pluripotent stem cells comprising: a) plating the pluripotent stem cells at a seeding density of 1-5×10⁶ cells per 60 mm dish and culturing them overnight to produce colonies of 10-100 cells, b) inducing differentiation of the cells generated in step (a) by incubating said cells for about 2 days in an induction media comprising ascorbic acid and a Wnt activator, c) removing the Wnt activator and continuing incubation for about 3 days, d) detaching cell layers formed in step (c) enzymatically and/or mechanically, and plating them onto matrix-coated dishes or onto a monolayer of feeder cells, e) culturing the cells plated in step (d) for about 12 days in alpha-MEM differentiation media comprising 10% FBS, ascorbic acid, SCF, IL-3, IL-7, IL-15, and FLT3-L, resulting in the formation of a three-dimensional vascular organoid which promotes differentiation of NK cells, f) collecting floating cells formed during step (e) and re-plating them onto matrix-coated dishes or onto a semi-confluent monolayer of feeder cells, g) culturing the replated cells of step (f) for about 10-16 days in maturation media comprising cytokines and the Wnt activator, but not comprising IL-3, to promote NK cell maturation, and h) optionally, purifying NK cells generated in step (g). In some embodiments, the purification of NK cells may be achieved by isolating CD56 cells.

In yet another aspect is provided a method for producing myeloid cells from pluripotent stem cells comprising: a) plating the pluripotent stem cells at a seeding density of 1-5×10⁶ cells per 60 mm dish and culturing them overnight to produce colonies of 10-100 cells, b) inducing differentiation of the cells generated in step (a) by incubating said cells for about 2 days in an induction media comprising ascorbic acid and a Wnt activator, c) removing the Wnt activator and continuing incubation for about 3 days, d) detaching cell layers formed in step (c) enzymatically and/or mechanically, and plating them onto matrix-coated dishes or onto a monolayer of feeder cells, e) culturing the cells plated in step (d) for about 12 days in alpha-MEM differentiation media comprising 10% FBS, ascorbic acid, and cytokines specific to a particular myeloid lineage, resulting in the formation of a three-dimensional vascular organoid which promotes differentiation of myeloid cells, f) collecting floating cells formed during step (e) and re-plating them onto matrix-coated dishes or onto a semi-confluent monolayer of feeder cells, g) culturing the replated cells of step (f) for about 7-14 days in maturation media comprising cytokines to promote myeloid cell maturation, and h) optionally, purifying the myeloid cells generated in step (g). In some embodiments, the purification of myeloid cells is achieved by isolating CD15+ or CD14+ or CD11b+ or CD33+ or CD235+ cells.

In some embodiments of the methods for producing NK cells disclosed herein, the cytokines present in the maturation media of step (g) may comprise SCF, IL-7, and/or IL-15.

In some embodiments of any of the methods described herein, in step (a) the cells may be cultured overnight in the presence of Fibroblast Growth Factor 2 (FGF2). In some embodiments, the FGF2 may be present at a concentration of 20-100 ng/mL. In some embodiments, step (c) may further comprise adding vascular endothelial growth factor (VEGF). In some embodiments, VEGF may be added at a concentration of 20-100 ng/ml. In some embodiments, the ascorbic acid may present in the induction media of step (b) at a concentration of 60 μg/mL.

In some embodiments of any of the methods for producing any of various cells disclosed herein, the Wnt activator may be selected from, for example, Wnt4 protein, CHIR99021 (CAS registry number 252917-06-9), SB-216763, BIO (6-bromoindirubin-3′-oxime), LY2090314, WAY-316606, ABC99, (hetero)arylpyrimidines, IQ1, QS11, Deoxycholic acid (DCA) and 2-amino-4-[3,4-(methylenedioxy)benzyl-amino]-6-(3-methoxyphenyl)pyrimidine. In certain embodiments, the Wnt activator may be CHIR99021 (CAS registry number 252917-06-9). In some embodiments, CHIR99021 may be present in the induction media of step (b) at a concentration of 3-8 μM. In various embodiments, the CHIR99021 may be present at a concentration of 6 μM.

In some embodiments of any of the methods for producing any of various cells disclosed herein, the feeder cells may be stromal cells. In some embodiments, the stromal cells may be OP9, OP9-Jagged2, OP9-DLL1, OP9-DLL3, or OP9-DLL4 cells.

In various embodiments, no cytokines may be present in steps (a)-(c).

In some embodiments, the pluripotent stem cells may be induced pluripotent stem cells (iPSCs) or embryonic stem cells (ESCs). In some embodiments, the pluripotent stem cells may be iPSCs. In some embodiments, the iPSCs may be generated from somatic cells. In some embodiments, the somatic cells may be peripheral blood mononuclear cells (PBMCs), peripheral blood natural killer cells (PBNKs), epithelial cells, fibroblasts, or adipocytes. In some embodiments, the iPSCs may be generated from endothelial cells, HPCs, lymphoid cells, or myeloid cells. In some embodiments, the iPSCs may be generated from NK cells.

In various embodiments, the iPSCs may be generated by the induction of expression of Oct3/4, Sox2, Klf4, and c-Myc. In various embodiments, the iPSCs may be generated by the induction of expression of Oct3/4, NANOG, Sox2, and Lin28.

In various embodiments, the methods for producing NK cells from pluripotent stem cells disclosed herein may further comprise expanding the NK cells produced in step (g) by culturing them in expansion media comprising IL-2. In some embodiments, the expansion media may comprise 50-300 U IL-2. In some embodiments, the expansion media may comprise RPMI-1640 supplemented with 5-10% FBS.

In various embodiments, the methods for producing NK cells from pluripotent stem cells disclosed herein may further comprise expanding NK cells produced in step (g) by culturing them in the presence of allogeneic feeder cells. In some embodiments, the allogeneic feeder cells may be cancer cells or their plasma membrane particles. In some embodiments, the allogeneic feeder cells may be irradiated K562 or membrane-bound IL15 or IL-21 expressing K562.

In some embodiments of any of the methods for producing any of various cells disclosed herein, the matrix-coated dishes may be coated with fibronectin, gelatin, or collagen.

In some embodiments of any of the methods for producing any of various cells or populations thereof of the present disclosure, step (d) may further comprise purifying CD31/CD34/CD144 triple-positive cells. In some embodiments, the purifying of the CD31/CD34/CD144 triple-positive cells may occur after the detaching step.

In another aspect is provided a method of suppressing expression of NKG2A gene in a natural killer (NK) cell comprising subjecting the NK cell or a progenitor stem cell used to generate the NK cell to a CRISPR-Cas editing system and a guide RNA (gRNA) comprising the sequence selected from SEQ ID NO: 1-4.

In another aspect is provided a method of transiently suppressing expression of NKG2A gene in a natural killer (NK) cell comprising administering to the NK cell or expressing in the NK cell a siRNA comprising the sequence selected from SEQ ID NO: 5-10.

In various embodiments of any of the methods of suppressing expression and/or transiently suppressing expression of NKG2A gene in a natural killer (NK) cell disclosed herein, the NK cell may be generated using any of the methods for producing NK cells from pluripotent stem cells of the present disclosure.

In one aspect is provided a natural killer (NK) cell produced by any of the methods for producing such cells from pluripotent stem cells of the present disclosure. In another aspect is provided a pharmaceutical composition comprising any of various NK cells of the present disclosure produced by any of the methods for producing such cells disclosed herein.

In one aspect is provided a population of HPCs produced by any of the methods for producing such cells from pluripotent stem cells of the present disclosure. In another aspect is provided a pharmaceutical composition comprising any of various population(s) of HPCs of the present disclosure produced by any of the methods for producing such cells disclosed herein.

In one aspect is provided a population of myeloid cells produced by any of the methods for producing such cells from pluripotent stem cells of the present disclosure. In another aspect is provided a pharmaceutical composition comprising any of various myeloid cells produced by any of the methods for producing such cells disclosed herein.

In yet another aspect is provided a method of treating a disease or disorder in a subject in need thereof comprising administering to the subject a therapeutically effective amount of the NK cells or the pharmaceutical composition disclosed herein. In some embodiments, the disease or the disorder is an infection, a cancer, an autoimmune disease, myocardial infarction/ischemia, or liver cirrhosis.

In various embodiments, the cancer may be a solid cancer. In some embodiments, the solid cancer may be a brain cancer. In some embodiments, the brain cancer may be a glioma or a glioblastoma.

In various embodiments, the cancer may be a lymphoma or a leukemia.

In various embodiments, the infection may be a bacterial infection or a viral infection. In some embodiments, the viral infection is SARS-COV-2.

In one aspect is provided a method for inducing elimination of senescent cells or cancer cells or virus-infected cells in a subject in need thereof comprising administering to the subject a therapeutically effective amount of the NK cells or their pharmaceutical compositions disclosed herein.

In another aspect is provided a method for treating cancer, or an autoimmune disease, or neutropenia or non-malignant blood disorder in a subject in need thereof comprising administering to the subject a therapeutically effective amount of the HPCs or their pharmaceutical compositions disclosed herein.

In still yet another aspect is provided a method for treating infection, or cancer, or an autoimmune disease, or neutropenia, or non-malignant blood disorder in a subject in need thereof comprising administering to the subject a therapeutically effective amount of the myeloid cells or their pharmaceutical composition disclosed herein.

These and other aspects described herein will be apparent to those of ordinary skill in the art in the following description, claims and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows generation of iPSC-derived hematopoietic progenitors. Hematopoietic colony forming unit (CFU) assay and corresponding cell morphologies: BFU—erythroid, M—macrophage, GM—granulocyte-macrophage, GEMM—granulocyte, erythroid, monocyte and megakaryocyte.

FIG. 2 shows an image of a vascular organoid. Hematopoietic progenitor cells expressing CD43 develop inside of a tubular vascular organoid formed by endothelial cells stained for VE-Cadherin.

FIG. 3 shows an image depicting blood flow inside of a vascular organoid. Hematopoietic progenitor cells expressing CD43 developed and flowed along the organoid vasculature.

FIG. 4 shows induced pluripotent stem cell (iPSC)-derived natural killer (NK) cell (iPSC-NK cell) definition markers as determined by flow cytometry analysis. These data demonstrate efficient production of NK cells (CD56⁺ CD16⁺ CD3⁻) from iPSCs utilizing various methods disclosed herein.

FIG. 5 shows functional markers of iPSC-derived NK cells (or iPSC-NK cells) as determined by flow cytometry analysis. Exemplar flow analysis data of iPSC-NK cells demonstrate expression of inhibitory receptors CD94, NKG2A; activating cytotoxicity receptor NKp46; ADCC receptor CD16; inhibitory killer immunoglobulin-like receptors (KIRs); Perforin.

FIG. 6 shows an image of the morphologic appearance of iPSC-derived NK cells. iPSC-derived NK cells expanded in vitro as cell clusters.

FIG. 7 shows cytotoxicity of iPSC-NK cells against K562 cells. Representative results of cytotoxicity assay demonstrate 70% of killed K562 cells at the 5:1 E:T (Effector:Target) ratio. Acute myeloid leukemia (AML) K562 leukemia cells were pre-stained using a PKH67 fluorescent cell linker kit. NK and K562 cells were combined at three different E:T ratios (5:1, 10:1, 25:1). Following 2 hours incubation, propidium iodide (PI) was added and the specimens were analyzed by flow cytometry.

FIG. 8 shows comparative cytotoxicity of iPSC-NK cells and peripheral blood (PB) NKs against SF8628 diffuse intrinsic pontine gliomas (DIPG) cells. An exemplar image (left) shows larger sized DIPG cells with predominately apoptotic appearance, and killed DIPG cells by fluorescence (PI) at the 5:1 E:T ratio. A line graph (right) shows percent NK cell killing of DIPG cells as determined by cytotoxicity assay across various E:T ratios.

FIG. 9 demonstrates cytotoxic penetration of glioblastoma multiforme (GBM) 6634 spheroid by iPSC NKs during 90 minute exposure. Live staining reveals the NK cells and apoptotic GBM cells are labeled with Annexin5.

FIG. 10 shows cell lysis of cancer cells upon 15 minute interaction with iPSC-NK cells. NK cells and apoptotic cancer cells are individually labeled. Dead cells are unstained.

FIG. 11 shows a schematic diagram of pGR-NKG2A-1 and pGR-NKG2A-2 vectors. 295-572 (base pair) bp—CBh promoter; 819-5090 bp—endonuclease SpCas9; 5121-5328 bp—PolyA Signal; 5552-6007 bp—f1 ori; 6289-6393 bp—bla promoter; 6394-7254 bp—bla; 7425-8013 bp ColE1 ori; 8075-8315 bp—U6 promoter; 8322-8419 bp—a guide RNA coding sequence.

FIG. 12 displays a graphical representation of cytotoxicity against senescent (SEN) cells. Cytotoxicity of modified NK inhibitory receptor CD94/NK group 2 member A (NKG2A) knockout (KO) NKs over wild type (NKG2A intact) iPSC-NK cells was revealed after 4 hr incubation with senescent IMR-90 fibroblasts at day 7 after 10 Gy irradiation. Cell death was measured by assessing cell viability using violet red staining to quantify viable fibroblast cells combined with propidium iodide (PI)-based viability assays. iPSC NK cells were stained in using CellTracker Green CMFDA (5-chloromethylfluorescein diacetate) dye.

FIG. 13 shows a iPSC-NK functional marker interface diagram. Activating cytotoxicity receptor NKp46; ADCC receptor CD16; inhibitory killer immunoglobulin-like receptors (KIRs); Perforin; Inhibitory receptor CD94, NKG2A.

FIG. 14 shows Fas ligand (FasL) activation in NK cells. Induction of FasL expression (CD178) was achieved via overnight incubation of NKs and DIPGs (SF8628). Both peripheral blood mononuclear cell (PBMC)- and induced pluripotent stem cell (iPSC)-derived NKs responded to the DIPG stimulus, as indicated by the elevated cell numbers expressing FasL in comparison to untreated cells.

FIG. 15 shows cytotoxicity of iPSC-NK cells against glioblastoma. Exemplary images show a GBM (6634) spheroid (left) that was eliminated (lysed) by overnight exposure to iPSC NKs (right).

FIG. 16 shows cytotoxicity of iPSC-NK cells against U251 glioma cells. Representative images show adherent U251 glioma cells (left) that are nearly 90% killed at 1 hr post-exposure at a 1:5 E:T ratio (right).

FIG. 17 shows hematopoietic stem cells (HSCs) created in a vascular organoid exhibited an engraftable signature, expressing CD34 and CD90 but not CD73.

FIG. 18 shows cytotoxicity of iPSC-NK cells against resistant breast carcinoma line BT-474 (ATCC HTB-20). The graph shows viability of BT-474 control and senescent cells after 24 hr exposure at a 1:1 and 1:5 E:T ratio. Viability was determined with neutral red stain release. Optical densities in the control wells were accepted 100%. The senescent cells were lysed by NK cells with a higher rate as compared to control.

FIG. 19 depicts a heat map showing top differentially expressed genes in iPSC derived NK cells compared to NK cells expanded from peripheral blood (PB-NK) from the same patient.

FIG. 20 depicts in vivo chronic myelogenous leukemia (CML) tumor burden with iPSC-NK treatment. Representative bioluminescence images showing CML tumor burden in the two groups of mice on day 4, day 11 and day 32 post tumor inoculation. iPSC-NK treatment was administered on day 4 post tumor injection. For each image, the treated group is on the left and the untreated control group is on the right. The images show that iPSC-NK treated mice are cleared from tumor and do not produce tumor-initiated bioluminescent signal (top panel). Tumor flux data collected from day 1 (inoculation) through day 11 post-inoculation show reduced tumor progression in mice that received iPSC-NK cell treatment as compared to control mice (bottom panel).

FIG. 21 depicts exemplary cytometric flow analysis of endothelial cells showing that 53% of the CD31 CD144 cells are negative for CD73 after CHIR99021 induction [58].

FIG. 22 depicts exemplary cytometric flow analysis showing the CD31⁺ CD34⁺ fraction at hematopoietic progenitor cells at time point 1 (Day 4-7 of differentiation) (HPC1) of CHIR99021 induction.

FIG. 23 shows a graph demonstrating that the percentage of human CD45 cells evaluated at week 8, week 12, week 13, and week 23 continued to increase. Error bars represent standard error of the mean (SEM) calculated for at least two independent experiments.

FIG. 24 depicts exemplary cytometric flow analysis evaluated at 8 weeks post injection, showing the percentage of human of CD45⁺ engrafted cells generated by utilizing a hematopoietic progenitor cells at time point 1 (HPC1) differentiation time point.

FIG. 25 depicts exemplary cytometric flow analysis evaluated at 21 weeks post injection, showing the percentage of human of CD45⁺ engrafted cells generated by utilizing the cells at a HPC1 differentiation time point.

FIG. 26 depicts exemplary cytometric flow analysis evaluated at 8 weeks post injection, showing the percentage of human CD45⁺ engrafted cells generated by utilizing hematopoietic progenitor cells at time point 2 (Day 7-10 of differentiation) (HPC2) differentiation time point.

FIG. 27 depicts exemplary cytometric flow analysis evaluated at 12 weeks post injection, showing the percentage of human CD45⁺ engrafted cells generated by utilizing hematopoietic stem cells at time point 3 (Day 14-18 of differentiation) (HPC3) differentiation time point.

FIG. 28 depicts exemplary cytometric flow analysis evaluated at 12 weeks post injection, showing the percentage of human CD45⁺ engrafted cells generated by utilizing human cord blood (hUSB).

FIG. 29 shows a phase-contrast photomicrograph (left) of Jurkat cells co-incubated with induced pluripotent stem cells (iPSC)-derived natural killer (NK) cells for 2 hours. Propidium iodide (PI) staining (right) shows a majority of Jurkat cells are non-viable (killed).

FIG. 30 demonstrates increased performance of three-dimensional (3D) organoid-derived iPSC-NK cells compared to two-dimensional (2D) derived iPSC-NKs. The organoid-derived iPSC-NK cells exhibited much higher cytotoxisity (70% vs. 30%) at much lower E:T (Effector:Target) ratio (50:1 vs. 5:1).

FIG. 31 shows an exemplary photomicrograph depicting fluorescent staining of iPSC-derived endothelial cells (ECs) cells seeded onto 3D-printed gelatin scaffolds. Fluorescent staining reveals calcein (green) and endothelial marker vascular endothelial (VE)-cadherin (red) with deoxyribonucleic acid (DNA) stain (4′,6-diamidino-2-phenylindole) DAPI (blue) overlay.

### DETAILED DESCRIPTION

### Definitions

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

The terminology used herein is for the purpose of describing particular embodiments only and is not intended to be limiting. As used herein, the indefinite articles “a”, “an” and “the” should be understood to include plural reference unless the context clearly indicates otherwise.

The terms “abrogate”, “abrogation” “eliminate”, or “elimination” of expression of a gene or gene product (e.g., RNA or protein) refers to a complete loss of the transcription and/or translation of a gene or a complete loss of the gene product (e.g., RNA or protein). Expression of a gene or gene product (e.g., RNA or protein) can be detected by standard art known methods such as those described herein, as compared to a control, e.g., an unmodified cell.

The terms “activation” or “stimulation” means to induce a change in their biologic state by which the cells (e.g., T cells and NK cells) express activation markers, produce cytokines, proliferate and/or become cytotoxic to target cells. All these changes can be produced by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T cell and/or NK cell proliferation and/or upregulation or downregulation of key molecules.

The term “allogeneic” refers to any material that is derived from a different individual of the same animal species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The terms “Cas9” “Cas9 protein” or “Cas9 molecule” refer to an enzyme from bacterial Type II CRISPR/Cas system responsible for DNA cleavage. Cas9 used herein also includes wild-type protein as well as functional and non-functional variants thereof.

The term “chimeric antigen receptor” or “CAR” as used herein is defined as a cell-surface receptor comprising an extracellular target-binding domain, a transmembrane domain and a cytoplasmic domain, comprising a lymphocyte activation domain and optionally at least one co-stimulatory signaling domain, all in a combination that is not naturally found together on a single protein. This particularly includes receptors wherein the extracellular domain and the cytoplasmic domain are not naturally found together on a single receptor protein. Chimeric antigen receptors may be introduced to lymphocytes such as T cells and natural killer (NK) cells.

The terms “CRISPR system,” “Cas system” or “CRISPR/Cas system” refer to a set of molecules comprising an RNA-guided nuclease or other effector molecule and a guide RNA (gRNA) molecule that together are necessary and sufficient to direct and effect modification of nucleic acid at a target sequence by the RNA-guided nuclease or other effector molecule. In some embodiments, a CRISPR system comprises a gRNA and a Cas protein, e.g., a Cas9 protein. In some embodiments, a CRISPR system comprises two or more gRNAs and a Cas protein, e.g., a Cas9 protein. Such systems comprising a Cas9 or modified Cas9 molecule are referred to herein as “Cas9 systems” or “CRISPR/Cas9 systems.” In one example, the gRNA molecule and Cas molecule may be complexed, to form a ribonuclear protein (RNP) complex.

The term “crRNA” when used in connection with a gRNA molecule, is a portion of the gRNA molecule that comprises a targeting domain and a region that interacts with a tracrRNA.

The term “differentiation” refers to a method of decreasing the potency or proliferation of a cell or moving the cell to a more developmentally restricted state.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like

The term “embryonic stem cell” refers to a pluripotent cell isolated from an embryo that is maintained in in vitro cell culture. Such cells may be rapidly dividing cultured cells isolated from cultured embryos that retain in culture the ability to give rise, in vivo, to any of various cell types that comprise the adult animal, including the germ cells. Embryonic stem cells may be cultured with or without feeder cells. Embryonic stem cells may be established from embryonic cells isolated from embryos at any stage of development, including blastocyst stage embryos and pre-blastocyst stage embryos. Embryonic stem cells may have a rounded cell morphology and may grow in rounded cell clumps on feeder layers. Embryonic stem cells are well known to a person of ordinary skill in the art.

The term “endothelial cell” refers to any of various cells that may be able to form a barrier, or line any of various organs and/or cavities, e.g., blood vessels. Not wishing to be bound by theory, endothelial cells may express any number of receptors on their surface such as, but not limited to, pattern recognition receptors (PRRs), which may recognize pathogen-associated molecular patterns (PAMPs) from microorganisms; secrete cytokines; and/or may release antimicrobial peptides.

The term “expansion” refers to the outcome of cell division and cell death.

The terms “express” and “expression” means allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular, or transmembrane.

The term “feeder cells” or “feeders” refers to cells of one type that are co-cultured with cells of a second type to provide an environment in which the cells of the second type can grow, as the feeder cells provide growth factors and nutrients for the support of the second cell type. The feeder cells are optionally from a different species as the cells they are supporting. For example, certain types of human cells, including stem cells, can be supported by primary cultures of mouse embryonic fibroblasts, or immortalized mouse embryonic fibroblasts. The feeder cells may typically be inactivated when being co-cultured with other cells by irradiation or treatment with an anti-mitotic agent such as mitomycin to prevent them from outgrowing the cells they are supporting. Feeder cells may include endothelial cells, stromal cells (e.g., epithelial cells or fibroblasts), and leukemic cells. A non-limiting example of a feeder cell type may be a human feeder, such as a human skin fibroblast. Yet another non-limiting example of a feeder cell type may be mouse embryonic fibroblasts (MEFs). In general, various feeder cells may be used in part to maintain pluripotency, direct differentiation towards a certain lineage and promote maturation to a specialized cell types, such as an effector cell.

The term “feeder-free” (FF) environment refers to an environment such as a culture condition, cell culture or culture media which is essentially free of feeder or stromal cells, and/or which has not been pre-conditioned by the cultivation of feeder cells. “Pre-conditioned” medium refers to a medium harvested after feeder cells have been cultivated within the medium for a period of time, such as for at least one day. Pre-conditioned medium contains many mediator substances, including growth factors and cytokines secreted by the feeder cells cultivated in the medium.

The term “gene editing nuclease” as used herein refers to a polypeptide or protein comprising one or more DNA-binding domains or components and one or more DNA-cutting domains or components. The term also encompasses isolated nucleic acids, e.g., one or more vectors, encoding said DNA-binding and DNA-nuclease domains or components. Gene editing nucleases are used for modifying the nucleic acid of a target gene and/or for modulating the expression of a target gene. For example, the one or more DNA-binding domains or components are associated with the one or more DNA-cutting domains or components, such that the one or more DNA-binding domains target the one or more DNA-cutting domains or components to a specific nucleic acid site. Gene editing nuclease that can be used in the present disclosure include but are not limited to, Clustered Regularly Interspaced Short Palindromic Repeats (CRISPR)/Cas nucleases, zinc finger nucleases (ZFN), transcription activator-like effector nucleases (TALENs), and meganucleases.

The term “granulocyte” refers to any of neutrophils, eosinophils, basophils or mast cells. Granulocytes may perform any number of functions such as, but not limited to, identification, ingestion, and/or destruction of microbial pathogens, e.g., via receptors, oxidative mechanisms, and/or enzymes, including lysozyme, collagenase, and elastase.

The terms “guide RNA”, “guide RNA molecule”, “gRNA molecule” or “gRNA” are used interchangeably, and refer to a set of nucleic acid molecules that promote sequence-specific targeting of an RNA-guided nuclease or other effector molecule (typically in complex with the gRNA molecule) to a target sequence. In some embodiments, targeting is accomplished through hybridization of a portion of the gRNA to DNA (e.g., through the gRNA targeting domain), and by binding of a portion of the gRNA molecule to the RNA-guided nuclease or other effector molecule (e.g., through at least the tracrRNA). In some embodiments, a gRNA molecule consists of a single contiguous polynucleotide molecule, referred to herein as a “single guide RNA” or “sgRNA” and the like. In other embodiments, a gRNA molecule consists of a plurality, usually two, polynucleotide molecules, which are themselves capable of association, usually through hybridization, referred to herein as a “dual guide RNA” or “dgRNA”, and the like. gRNA molecules are described in more detail below, but generally include a targeting domain and a tracrRNA. In some embodiments the targeting domain and tracrRNA are disposed on a single polynucleotide. In other embodiments, the targeting domain and tracr are disposed on separate polynucleotides.

The term “hematopoietic cell” refers to any of various blood cells, such as, but not limited to, e.g., myeloid and lymphoid cell types including macrophages, erythrocytes, neutrophils, T cells, natural killer cells, and B cells.

The term “hematopoietic progenitor cell” or “HPC” as used herein, refers to progenitor cells relating to or involved in the formation of blood cells, including hematopoietic stem cells.

The term “hematopoietic stem cell” or “HSC” as used herein, refers to stem cells capable of self-renewal and giving rise to both committed myeloid and committed lymphoid progenitors.

The term “hemogenic endothelium” refers to a specialized subset of endothelial cells that possess or acquire blood-forming potential.

The term “induced pluripotent stem cells” or, iPSCs, means that the stem cells are produced from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature.

The term “modify” or “modification” when used in connection with a nucleic acid, e.g., a target sequence, refers to a chemical difference at or near the target sequence relative to its natural state. In some embodiments, a modification comprises an insertion and/or deletion of one or more nucleotides. In some embodiments, a modification comprises a DNA strand break (e.g., a double strand DNA break).

The term “monocyte” or “macrophage” refers to any of various cells that may have the capacity to remove substances from the body, e.g., via phagocytosis or ingestion. While not wishing to be bound by theory, monocytes that may circulate in blood may migrate to an inflammatory site and transform themselves into macrophages. Macrophages may have any number of different functions comprising, e.g., i) they may be phagocytic and/or exhibit a microbicidal mechanism such as through oxygen-dependent or -independent mechanism(s); ii) they may be able to present antigen(s) and/or activate lymphocyte(s); iii) they may release and/or stimulate cytokine secretion; iv) they may modulate an immune response; v) they may participate in tissue reorganization such as that which may occur with an inflammation process via production of extracellular matrix proteins (e.g., collagen or elastase) and/or matrix metalloproteinases; and/or vi) they may produce cytotoxic factors such as those that may be involved in immunity against tumors. Based on the biological function, there are three populations of macrophages: i) classically activated macrophages or type 1-activated macrophages; ii) alternatively activated macrophages; and iii) type 2-activated macrophages.

The terms “natural killer cell” and “NK cell” are used interchangeably and synonymously herein. NK cell may refer to a differentiated lymphocyte with a CD3− CD16+, CD3− CD56+, CD16+ CD56+ and/or CD57+ TCR− phenotype. NKs may be characterized by their ability to bind to and kill cells that fail to express “self” MHC/HLA antigens by the activation of specific cytolytic enzymes, the ability to kill tumor cells or other diseased cells that express a ligand for NK activating receptors, and/or the ability to release protein molecules called cytokines that stimulate or inhibit the immune response.

The term “NKG2A” refers to a form of a C-type lectin receptor, which may be expressed e.g., on the surface of natural killer (NK) cells. These receptors may stimulate or inhibit cytotoxic activity of NK cells, and therefore they are divided into activating and inhibitory receptors according to their function. In some embodiments described herein, a population of cells comprising NK cells is provided, wherein NK cells are modified such that they lack expression of an NK inhibitory molecule or manifest a reduced expression of an NK inhibitory molecule. In some embodiments, the NK inhibitory molecules is a form of a C-type lectin receptor. In some embodiments, the NK inhibitory molecule is NKG2A.

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers.

The term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell), which is unable to give rise to a complete organism to the more primitive, more pluripotent cell, which is able to give rise to a complete organism (e.g., an embryonic stem cell).

The term “pluripotent stem cell” refers to an unspecialized cell, i.e. not fixed as to developmental potential, capable of differentiation into any one of various cell types. Non-limiting examples of pluripotent stem cells are embryonic stem cells and induced pluripotent stem cells (iPSCs).

The term “polypeptide,” “peptide” or “protein” are used interchangeably and to refer to a polymer of amino acid residues. The terms encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The term “proliferation” refers to an increase in cell division, either symmetric or asymmetric division of cells.

The terms “purify” “purified”, “purification” or equivalents thereof as defined herein and with regard to preparations of cells, e.g., a cell culture, cell sample, or cell population, mean to separate cells from attendant material or to separate from material considered to be undesirable. In some embodiments, the purified cell culture, cell sample, or cell population may comprise at least about 1%, 2%, 3%, 4%, 5%, 6%, 7%, 8%, 9%, 10%, 30%, 40%, 50%, 55%, 60%, 65%, 70%, 75%, 80%, 85%, 90%, 95%, or 100% of a desired cell lineage or a desired cell having a certain cell phenotype, e.g., expressing a certain cell marker or not expressing a certain cell marker gene characteristic of that cell phenotype. The terms “purify” “purified”, “purification” or equivalents thereof as used herein do not necessarily indicate the presence of only one type of cell. Rather, a purified cell culture, cell sample, or cell population may include, for example, any of various cell types disclosed herein, among others. As a non-limiting example, purification of a cell culture, cell sample, or cell population may comprise removal of one or more undesirable ingredient(s) of a media used in cell culturing methods, including serum, buffer, cytokines, and the like, from the cells. In various embodiments, the purification of HPCs disclosed herein may be achieved, for example, by isolating CD34+ cells. In various embodiments, the purification of NK cells disclosed herein may be achieved, for example, by isolating CD56+ cells.

The terms “reduce”, “reduction”, or “decrease” of expression of a gene or gene product (e.g., RNA or protein) refer to an overall decrease of at least 25%, 30%, 40%, 50%, 60%, 70%, 80%, 81%, 82%, 83%, 84%, 85%, 86%, 87%, 88%, 89%, 90%, 91%, 92%, 93%. 94%, 95%, 96% 97%, 98% or 99% up to 100% (abrogation or elimination) in the transcription and/or translation of a gene or in the levels of the gene product (e.g., RNA or protein).

The term “targeting domain” when used in connection with a gRNA, is the portion of the gRNA molecule that recognizes, e.g., is complementary to, a target sequence, e.g., a target sequence within the nucleic acid of a cell, e.g., within a gene.

The term “target sequence” refers to a sequence of nucleic acids complementary, for example fully complementary or partially complementary, to a gRNA targeting domain. In some embodiments, the target sequence is disposed on genomic DNA. In an embodiment the target sequence is adjacent to (either on the same strand or on the complementary strand of DNA) a protospacer adjacent motif (PAM) sequence recognized by a protein having nuclease or other effector activity, e.g., a PAM sequence recognized by Cas9. In embodiments, the target sequence is a target sequence in a gene encoding an endogenous HLA class I molecule or HLA class II molecule

The term “tracrRNA” when used in connection with a gRNA molecule, refers to the portion of the gRNA that binds to a nuclease or other effector molecule. In some embodiments, the tracrRNA comprises a nucleic acid sequence that binds specifically to Cas9. In some embodiments, the tracrRNA comprises a nucleic acid sequence that binds specifically to a crRNA.

The term “transduction” means the introduction of a foreign nucleic acid into a cell using a viral vector.

The term “transfection” means the introduction of an extrinsic or extracellular nucleic acid into a cell using recombinant DNA technology.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician

The term “vascular organoid” refers to a vessel-like structure (tube) of cells capable of forming hematopoietic progenitor cells.

The terms “vector”, “cloning vector” and “expression vector” mean the vehicle by which a DNA or RNA sequence (e.g., a foreign gene) can be introduced into a host cell, so as to modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alpha viral, herpes, lentiviral, retroviral, or vaccinia vector.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The practice of the present disclosure employs, unless otherwise indicated, conventional techniques of statistical analysis, molecular biology (including recombinant techniques), virology, microbiology, cell biology, chemistry and biochemistry, which are within the skill of the art. Such tools and techniques are described in detail in e.g., Sambrook, Fritsch & Maniatis, Molecular Cloning: A Laboratory Manual, Second Edition. Cold Spring Harbor, NY: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nucl. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

### Stem Cells

In certain aspects is provided stem cells, that may be cultured and maintained using of various methods described herein. Stem cells are a basic type of undifferentiated cell that may divide and give rise to any type of cell in the body or self-renew. Stem cells may comprise pluripotent stem cells, which may develop into primary germ cell layers of the embryo, or totipotent stem cells, which have the capacity to form an entire organism. Stem cells may give rise to various other cells types that may possess specialized functions e.g., multipotent stem cells, which may develop into more than one specific type of cells that may develop to form terminally differentiated cells for all different types of tissues in the body. Non-limiting examples of cells that may be generated, cultured, maintained and/or differentiated using any of the methods described herein may be hematopoietic stem cells (HSCs), lymphoid progenitor cells, totipotent cells, or any of various cells that may produce one or more cell types, e.g., NK progenitor. In some embodiments, stem cells of the present disclosure may be maintained in culture in an undifferentiated state for genetic manipulation and unlimited expansion.

### Pluripotent Stem Cells

In certain aspects is provided pluripotent stem cells, e.g. human embryonic stem cells (hESCs) and iPSCs, that may be cultured and maintained using a variety of methods as disclosed herein. Non-limiting examples of hESCs are H1, H9, hES2, hES3, hES4, hES5, hES6, BG01, BG02, BG03, HSF1, HSF6, H1, H7, H9, H13B, and/or H14 etc. In certain aspects, the iPSCs may be derived from various cell types such as, but not limited to, peripheral blood mononuclear cells (PBMCs), peripheral blood natural killer cells (PBNKs), skin (epithelial) cells, fibroblasts, and fat cells (adipocytes). In certain aspects, the iPSCs may be derived from somatic cells.

In some embodiments, the iPSCs may be derived from peripheral blood mononuclear cells (PBMCs). In some embodiments, the pluripotent cells may be derived from peripheral blood natural killer cells (PBNKs). In some embodiments, the pluripotent stem cells may be derived from NK cells. In some embodiments, the pluripotent stem cells may be derived from enriched NK cells.

In some embodiments, the iPSCs may be produced by the induction of expression of any number of embryonic genes. Non-limiting examples of embryonic genes are Oct3/4 (Pou5f1), Sox2, Klf4, c-Myc, NANOG, SOX2 and LIN28 (ONSL). In some embodiments, the expression of embryonic genes may be induced by any number of chemical stimuli such as, but not limited to inhibitors of TGF-β receptor and MEK, e.g., SB-431542 and PD0325901.

In some embodiments, the pluripotent stem cells may be cultured and maintained in the presence of feeder cells. As a non-limiting example, the feeder cells may be mouse embryonic fibroblasts (MEFs). As another non-limiting example, the feeder cells may be OP9-DLL4 cells. In some embodiments, the pluripotent stem cells may be cultured and maintained in the absence of feeder cells using a feeder-free or feeder-independent culture system.

In some embodiments, various matrix components may be used as a substrate for culturing and maintaining pluripotent stem cells disclosed herein. Non-limiting examples of such matrix components include collagen IV, fibronectin, laminin, and vitronectin, Geltrex™, CellBIND®. Matrigel™ may also be used to provide a substrate for cell culture and maintenance of pluripotent stem cells.

Cord blood stem cells are considered superior to bone marrow stem cells in terms of risks of rejection, contamination, and infection. They may also outperform bone marrow in their ability to replace cells damaged or deceased from chemotherapy or radiation treatments. Depending on ethnic background, the chances of finding a cord blood match range between 29% and 79%. Moreover, the quantity of cord blood stem cells may not be adequate for treatment of adults as the stem cell yield from 100 to 200 ml of cord blood would suffice the requirement of only a 10 kg child. Only 8-10% of units will have sufficient volume for an adult. The slow engraftment rates and high cost are other disadvantages. The chance estimate that a person will require autologous transfusion is between 1 in 1,000 to 1 in 200,000. At present, there are no data on the long-term viability of cord blood cells. The storage is expensive, and the quantity may not be sufficient for use in older children or adults [59]. There is an unmet need for new sources of hematopoietic stem cells (HSCs). iPSCs may provide a source of autologous HSCs derived from adult patient cells.

In some embodiments, somatic cells may be collected from a subject, e.g., a human patient, and optionally stored, e.g., in a cell bank, for subsequent derivation of iPSCs useful in the practice of any of the methods of the present disclosure. In some embodiments, iPSCs may be derived from the somatic cells to produce any of various cells disclosed herein, e.g., hemogenic endothelium, hematopoietic progenitor cells (HPCs), hematopoietic stem cells, natural killer (NK) cells, and/or myeloid cells, which may be produced using any of the vascular organoid-based differentiation systems disclosed herein. Such cells may be useful in the practice of any of the methods of treating a disease or disorder in a subject disclosed herein. In some embodiments, such cells may be useful in treating the same subject from which the somatic cells were collected, and may be autologous to the subject undergoing the treatment. In some embodiments, such cells may be useful in treating a different subject from which the somatic cells were, and may be allogenic to the subject undergoing the treatment. In some embodiments, the subject from which the somatic cells are collected may be healthy. In some embodiments, the subject from which the somatic cells are collected may be in need of treatment for a disease or disorder. As a non-limiting example, the subject from which the somatic cells are collected may be in need of cellular therapies while undergoing chemotherapy, e.g., myeloblative chemotherapy.

In some embodiments, methods for a cell culture system for differentiating stem cells disclosed herein may be useful for producing any of various cells of the present disclosure for storage in a cell bank. As a non-limiting example, engraftable hematopoietic progenitor cells (HPC) and/or hematopoietic stem cells (HSC) derived from human pluripotent stem cells (hPSCs) using cell differentiation methods disclosed herein may be stored in a cell bank. Stored cells may optionally be further differentiated using any of the methods of the present disclosure.

### Engraftable Cells

In certain aspects is provided engraftable cells, e.g., engraftable hemogenic endothelium, hematopoietic progenitor cells (HPCs), and/or hematopoietic stem cells (HSCs), and methods for producing such engraftable cells from any of the stem cells or pluripotent stem cells disclosed herein. In some embodiments, engraftment may refer to any process whereby any of various transplanted cells and/or tissues may be incorporated and/or retained. Without wishing to be bound by theory, a non-limiting example of long term engraftment may be recognized when donor hematopoietic cells are registered over period of 2-4 month after HPC/HSC transplant. In some embodiments, an engraftable cell may be distinguished by expression, or lack thereof, of any of various markers or combinations thereof, thereby giving rise to an engraftable cell signature. Such markers may be determined using conventional methodology by those skilled in the art, e.g., by flow cytometry or reverse transcription polymerase chain reaction (rtPCR) approaches. As a non-limiting example, an engraftable cell signature may comprise marker expression of CD34⁺CD90⁺CD73⁻ as determined by flow cytometry. As another non-limiting example, an engraftable cell signature may comprise marker expression of CD34⁺CD31⁺CD144⁺CD43⁻CD45⁻CD73⁻RUNX1⁺ by rt-PCR. As a non-limiting example, an engraftable cell signature may comprise marker expression of CD34⁺CD90⁺CD73⁻ as determined by flow cytometry and marker expression of CD34⁺CD31⁺CD144⁺CD43⁻CD45⁻CD73⁻RUNX1⁺ by rt-PCR.

In certain aspects is provided various populations of hematopoietic progenitor cells (HPCs) and methods for producing the hematopoietic progenitor cell populations from any of the stem cells or pluripotent stem cells disclosed herein to generate e.g., pluripotent stem cell-derived hematopoietic progenitor cells (HPCs).

In certain aspects, the method for producing a population of HPCs from pluripotent stem cells may comprise: a) plating the pluripotent stem cells at a seeding density of 1-5×10⁶ cells per 60 mm dish and culturing them overnight to produce colonies of 10-100 cells, b) inducing differentiation of the cells generated in step (a) by incubating said cells for about 2 days in an induction media comprising ascorbic acid and a Wnt activator, c) removing the Wnt activator and continuing incubation for about 4-7 days to produce a first population of hematopoietic progenitor cells, and d) optionally, purifying the first population of hematopoietic progenitor cells generated in step (c). In some embodiments, the first population of HPCs may comprise engraftable HPCs.

In certain aspects, any of the above-described methods for producing a population of HPCs may further comprise: e) detaching cell layers formed in step (c) enzymatically and/or mechanically, and plating them onto matrix-coated dishes or onto a monolayer of feeder cells, f) culturing the cells plated in step (e) for about 2-5 days in alpha-MEM differentiation media comprising e.g., 10% FBS, ascorbic acid, and/or one or more cytokines, resulting in the formation of a second population of hematopoietic progenitor cells, and g) optionally, purifying the second population of hematopoietic progenitor cells generated in step (f). In some embodiments, the second population of HPCs may comprise engraftable HPCs.

In certain aspects, any of the above-described methods for producing a population of HPCs may further comprise: e) detaching cell layers formed in step (c) enzymatically and/or mechanically, and plating them onto matrix-coated dishes or onto a monolayer of feeder cells, f) culturing the cells plated in step (e) for about 9-13 days in alpha-MEM differentiation media comprising e.g., 10% FBS, ascorbic acid, and/or one or more cytokines, resulting in the formation of a third population of hematopoietic progenitor cells from a three-dimensional vascular organoid, and g) optionally, purifying the third population of hematopoietic progenitor cells generated in step (f). In some embodiments, the third population of HPCs may comprise engraftable HPCs.

In various embodiments of the present disclosure, the first population of HPCs is also referred to as HPC1, the second population of HPCs is also referred to as HPC2, and the third population of HPCs is also referred to as HPC3. The first population of HPCs or HPC1 may include hematopoietic progenitor cells that arise during the monolayer hematopoietic induction method as described herein and express CD34. The second population of HPCs or HPC2 may include hematopoietic progenitor cells that arise after the progenitors obtained by the monolayer induction method as described herein are conditioned in hemogenic conditions, containing cytokines for hematopoietic stem cell differentiation. In some embodiments, HPC2 may express CD34. In some embodiments, HPC2 may not express CD34. The third population of HPCs or HPC3 may include hematopoietic progenitor cells that arise from the vascular organoid grown in media containing cytokines for hematopoietic stem cell differentiation. In some embodiments, HPC3 may express CD34. In some embodiments, HPC3 may not express CD34.

In some embodiments, the method for producing populations of HPCs may comprise plating pluripotent stem cells and culturing them in the presence of a growth factor such as, but not limited to, Fibroblast Growth Factor 2 (FGF2). As a non-limiting example, the FGF2 may be present at a concentration of 20-100 ng/mL. In some embodiments, the method for producing HPCs may comprise plating pluripotent stem cells and culturing them in absence of a growth factor.

In some embodiments, the plating of the pluripotent stem cells may be at a seeding density of 1-5×10⁶ cells per 60 mm dish. As a non-limiting example, the plating of the pluripotent stem cells may be at a seeding density of 1×10⁶ cells per 60 mm dish, 2×10⁶ cells per 60 mm dish, 3×10⁶ cells per 60 mm dish, 4×10⁶ cells per 60 mm dish, or 5×10⁶ cells per 60 mm dish. In certain embodiments, the pluripotent stem cells may be cultured for any period of time. As a non-limiting example, the pluripotent stem cells may be cultured overnight. In certain embodiments, the pluripotent stem cells may be cultured for any period of time, e.g., overnight, to produce colonies of cells comprising any number of cells such as, but not limited to, 10-100 cells. As a non-limiting example, the colonies of cells may comprise 10 cells, 12 cells, 14 cells, 16 cells, 18 cells, 20 cells, 22 cells, 24 cells, 26 cells, 28 cells, 30 cells, 32 cells, 34 cells, 36 cells, 38 cells, 40 cells, 42 cells, 44 cells, 46 cells, 48 cells, 50 cells, 52 cells, 54 cells, 56 cells, 58 cells, 60 cells, 62 cells, 64 cells, 66 cells, 68 cells, 70 cells, 72 cells, 74 cells, 76 cells, 78 cells, 80 cells, 82 cells, 84 cells, 86 cells, 88 cells, 90 cells, 92 cells, 94 cells, 96 cells, 98 cells, or 100 cells.

In some embodiments, the colonies of cells may be induced to differentiate by any one of various methods disclosed herein. In some embodiments, the colonies of cells may be induced to differentiate by incubating said cells for any number of days in an induction media. In some embodiments, the colonies of cells may be induced to differentiate by incubating said cells for about 2 days in an induction media. In some embodiments, the induction media may comprise ascorbic acid. In certain embodiments, the ascorbic acid may be present in the induction media at the concentration of 60 μg/mL. In some embodiments the induction media may comprise a Wnt activator. In some embodiments, the induction media may comprise ascorbic acid and a Wnt activator. In certain embodiments, the Wnt activator may be a Wnt protein such as, but not limited to a Wnt4 protein. In certain embodiments, the Wnt activator may target Glycogen synthase kinase-3 (GSK-3). Non-limiting examples of Wnt activators that may target GSK-3 include SB-216763 (Coghlan et al., 2000 [31]), CHIR99021 (PubChem), BIO (6-bromoindirubin-3′-oxime) (Sato et al, 2004 [32]), and LY2090314 (Atkinson et al., 2015 [33]). In certain embodiments, the Wnt activator may target any one of Secreted frizzled-related protein (SFRP), Notum, Protein phosphatase 2 (PP2A), ADP-ribosylation factor GTPase-activating protein 1 (ARFGAP1), and beta-catenin. Non-limiting examples of such Wnt activators may include WAY-316606 (Bodine et al., 2009 [34]), ABC99 (Suciu et al, 2018 [35]), (hetero)arylpyrimidines (Gilbert et al., 2009 [36]), IQ-1 (Miyabayashi et al., 2007 [37]), QS11 (Zhang et al., 2007 [38]), DCA (Pai et al., 2004 [39]), and 2-amino-4-[3,4-(methylenedioxy)benzyl-amino]-6-(3-methoxyphenyl)pyrimidine (Liu et al., 2005 [40]). In some embodiments, the Wnt activator may be removed from the media.

In some embodiments, vascular endothelial growth factor (VEGF) may be added to the culture media. In certain embodiments, VEGF may not be added to the culture media.

In some embodiments, the cells generated using any of the methods described above may be further incubated for a period of time such as, but not limited to, about 3 days to produce, for example, hematopoietic progenitor cells. In some embodiments, the hematopoietic progenitor cells may form a cell layer. In some embodiments, the layer of hematopoietic cells may be detached either enzymatically or mechanically. In some embodiments, the layer of hematopoietic progenitor cells may comprise CD31/CD34/CD144 triple-positive cells that may be optionally purified using conventional methodology by those skilled in the art.

In some embodiments, the hematopoietic cells may be plated onto a matrix-coated dish.

In some embodiments, the matrix coated dish may be coated with, as an example, fibronectin, gelatin or collagen.

In some embodiments, the hematopoietic cells may be plated onto a fibronectin coated dishes. In some embodiments, the hematopoietic cells may be plated onto a monolayer of feeder cells. The monolayer of feeder cells may be confluent. The monolayer of feeder cells may be non-confluent e.g., semi-confluent. The monolayer of feeder cells may be about 60% confluent to overconfluent.

In some embodiments, the plated cells, e.g., HPCs, may be cultured for any number of days, e.g., for about 2-5 days or for about 9-13 days, in a differentiation media, said differentiation media may comprise alpha-MEM differentiation media comprising e.g., 10% FBS, ascorbic acid, SCF, IL-3, and TPO or a different combination of cytokines specific for HPC differentiation. In some embodiments, the differentiation media may comprise, for example, SCF, IL-3, and TPO. In some embodiments, the differentiation media may comprise 10% FBS, ascorbic acid, SCF, IL-3, and TPO or a different combination with or without IL-3. In some embodiments, the differentiation media does not comprise cytokines and no cytokines are present.

In some embodiments, any of the various method steps of culturing, preparing, and/or purifying any of the cells disclosed herein may exclude cytokines such that no cytokines are present. As non-limiting example, steps (a)-(c) of any of the above disclosed methods may exclude cytokines such that no cytokines are present.

In some embodiments, the plated cells, e.g., HPCs, may be cultured for at least about 1 day, at least about 2 days, at least about 3 days, at least about 4 days, at least about 5 days, at least about 6 days, at least about 7 days, at least about 8 days, at least about 9 days, at least about 10 days, at least about 11 days, or at least about 12 days in a differentiation media. In some embodiments, the plated hematopoietic cells may be cultured for 2-5 in differentiation media. In some embodiments, the plated hematopoietic cells may be cultured for 9-13 in differentiation media.

In some embodiments, the culturing of the plated cells for about 2-5 days in a differentiation media comprising, e.g., 10% FBS, ascorbic acid, one or more cytokines, such as, but not limited to SCF, IL-3, and TPO, may result in the formation of a second population of HPCs.

In some embodiments, the second population of HPCs may be optionally purified. In some embodiments, the purification of the second population of HPCs may be achieved by isolating CD34+ cells. In some embodiments, the second population of HPCs may be engraftable.

In some embodiments, the culturing of the plated cells for about 9-13 days in a differentiation media comprising, e.g., 10% FBS, ascorbic acid, one or more cytokines, such as, but not limited to, SCF, IL-3, and TPO, may result in the formation of a third population of HPCs from a three-dimensional vascular organoid disclosed herein. In some embodiments, the purification of the third population of HPCs may be achieved by isolating CD34+ cells. In some embodiments, the third population of HPCs may be optionally purified. In some embodiments, the third population of HPCs may be engraftable.

In some embodiments, the culturing of the hematopoietic cells with the differentiation media results in the formation of a three-dimensional organoid. In some embodiments, the three-dimensional vascular organoid may promote differentiation of the hematopoietic cells disclosed herein to form hematopoietic progenitor cells, e.g., pluripotent stem cell-derived hematopoietic progenitor cells of the present disclosure. In some embodiments, the cells differentiated using the three-dimensional vascular organoid may be floating cells. In some embodiments, the floating cells differentiated using the three-dimensional vascular organoid may be collected and replated onto a matrix-coated dish. In some embodiments, the floating cells differentiated using the three-dimensional vascular organoid may be collected and replated onto a monolayer of feeder cells which may be confluent. In some embodiments, the floating cells differentiated using the three-dimensional vascular organoid may be collected and replated onto a monolayer of feeder cells which may be non-confluent, e.g. semi-confluent. In some embodiments, the replated floating cells may be cultured for any number of days such as, but not limited to, about 2-7 days. In some embodiments the replated floating cells may be cultured for any number of days in a maturation media. In some embodiments the replated floating cells may be cultured 2-7 days in a maturation media. The maturation media may comprise cytokines such as, but not limited to, SCF, IL-3, and TPO. The maturation media may comprise any cytokines of the present disclosure, but may not comprise IL-3. In some embodiments, the maturation media may comprise cytokines and any number of various Wnt activators disclosed herein. In some embodiments, the maturation media may comprise cytokines but not comprise IL-3, and any number of various Wnt activators disclosed herein. As a non-limiting example, the maturation media may comprise SCF and a Wnt activator disclosed herein, but may not comprise IL-3. In some embodiments, the maturation media may promote HPC maturation thereby producing HPCs derived from pluripotent stem cells, i.e., pluripotent stem cell-derived HPCs.

In some embodiments of any of the above methods for producing pluripotent stem cell-derived HPCs, the FGF2 may be present in the induction media at the concentration of 20-100 ng/mL. As a non-limiting example, the FGF2 may be present in the induction media at a concentration of 20 ng/mL, 22 ng/mL, 24 ng/mL, 26 ng/mL, 28 ng/mL, 30 ng/mL, 32 ng/mL, 34 ng/mL, 36 ng/mL, 38 ng/mL, 40 ng/mL, 42 ng/mL, 44 ng/mL, 46 ng/mL, 48 ng/mL, 50 ng/mL, 52 ng/mL, 54 ng/mL, 56 ng/mL, 58 ng/mL, 60 ng/mL, 62 ng/mL, 64 ng/mL, 66 ng/mL, 68 ng/mL, 70 ng/mL, 72 ng/mL, 74 ng/mL, 76 ng/mL, 78 ng/mL, 80 ng/mL, 82 ng/mL, 84 ng/mL, 86 ng/mL, 88 ng/mL, 90 ng/mL, 92 ng/mL, 94 ng/mL, 96 ng/mL, 98 ng/mL, or 100 ng/mL.

In some embodiments of any of the above methods for producing pluripotent stem cell-derived HPCs, the ascorbic acid may be present in the induction media at the concentration of 20-100 μg/mL. As a non-limiting example, the ascorbic acid may be present in the induction media at a concentration of 20 μg/mL, 22 μg/mL, 24 μg/mL, 26 μg/mL, 28 μg/mL, 30 μg/mL, 32 μg/mL, 34 μg/mL, 36 μg/mL, 38 μg/mL, 40 μg/mL, 42 μg/mL, 44 μg/mL, 46 μg/mL, 48 μg/mL, 50 μg/mL, 52 μg/mL, 54 μg/mL, 56 μg/mL, 58 μg/mL, 60 μg/mL, 62 μg/mL, 64 μg/mL, 66 μg/mL, 68 μg/mL, 70 μg/mL, 72 μg/mL, 74 μg/mL, 76 μg/mL, 78 μg/mL, 80 μg/mL, 82 μg/mL, 84 μg/mL, 86 μg/mL, 88 μg/mL, 90 μg/mL, 92 μg/mL, 94 μg/mL, 96 μg/mL, 98 μg/mL, or 100 μg/mL.

In some embodiments of any of the above methods for producing pluripotent stem cell-derived HPCs, the ascorbic acid may be present in the induction media at the concentration of 60 μg/mL.

In some embodiments of any of the above methods for producing pluripotent stem cell-derived HPCs, the Wnt activator may be CHIR99021 (CAS registry number 252917-06-9). In some embodiments, the CHIR99021 may be present in the induction media disclosed herein. In some embodiments, the CHIR99021 may be absent from the induction media disclosed herein. In some embodiments, the CHIR99021 may present in the induction media at a concentration of about 3-8 μM. As a non-limiting example, the CHIR99021 may be present in the induction media at a concentration of about 3 μM, 4 μM, 5 μM, 6 μM, 7 μM, or 8 μM. In some embodiments, the CHIR99021 may be present in the indication mediate at a concentration of 6 μM.

In some embodiments of any of the above methods for producing pluripotent stem cell-derived HPCs, vascular endothelial growth factor (VEGF) may be added to the culture media. As a non-limiting example, VEGF may be added to the culture media at a concentration of 20-100 ng/mL. In certain embodiments, the VEGF may be added to the culture media at a concentration of 20 ng/mL. In certain embodiments, the VEGF may be added to the culture media at a concentration of 22 ng/mL. In certain embodiments, the VEGF may be added to the culture media at a concentration of 24 ng/mL. In certain embodiments, the VEGF may be added to the culture media at a concentration of 26 ng/mL. In certain embodiments, the VEGF may be added to the culture media at a concentration of 28 ng/mL. In certain embodiments, the VEGF may be added to the culture media at a concentration of 30 ng/mL. In certain embodiments, the VEGF may be added to the culture media at a concentration of 32 ng/mL. In certain embodiments, the VEGF may be added to the culture media at a concentration of 34 ng/mL. In certain embodiments, the VEGF may be added to the culture media at a concentration of 36 ng/mL. In certain embodiments, the VEGF may be added to the culture media at a concentration of 38 ng/mL. In certain embodiments, the VEGF may be added to the culture media at a concentration of 40 ng/mL. In certain embodiments, the VEGF may be added to the culture media at a concentration of 42 ng/mL. In certain embodiments, the VEGF may be added to the culture media at a concentration of 44 ng/mL. In certain embodiments, the VEGF may be added to the culture media at a concentration of 46 ng/mL. In certain embodiments, the VEGF may be added to the culture media at a concentration of 48 ng/mL. In certain embodiments, the VEGF may be added to the culture media at a concentration of 50 ng/mL. In certain embodiments, the VEGF may be added to the culture media at a concentration of 52 ng/mL. In certain embodiments, the VEGF may be added to the culture media at a concentration of 54 ng/mL. In certain embodiments, the VEGF may be added to the culture media at a concentration of 56 ng/mL. In certain embodiments, the VEGF may be added to the culture media at a concentration of 58 ng/mL. In certain embodiments, the VEGF may be added to the culture media at a concentration of 60 ng/mL. In certain embodiments, the VEGF may be added to the culture media at a concentration of 62 ng/mL. In certain embodiments, the VEGF may be added to the culture media at a concentration of 64 ng/mL. In certain embodiments, the VEGF may be added to the culture media at a concentration of 66 ng/mL. In certain embodiments, the VEGF may be added to the culture media at a concentration of 68 ng/mL. In certain embodiments, the VEGF may be added to the culture media at a concentration of 70 ng/mL. In certain embodiments, the VEGF may be added to the culture media at a concentration of 72 ng/mL. In certain embodiments, the VEGF may be added to the culture media at a concentration of 74 ng/mL. In certain embodiments, the VEGF may be added to the culture media at a concentration of 76 ng/mL. In certain embodiments, the VEGF may be added to the culture media at a concentration of 78 ng/mL. In certain embodiments, the VEGF may be added to the culture media at a concentration of 80 ng/mL. In certain embodiments, the VEGF may be added to the culture media at a concentration of 82 ng/mL. In certain embodiments, the VEGF may be added to the culture media at a concentration of 84 ng/mL. In certain embodiments, the VEGF may be added to the culture media at a concentration of 86 ng/mL. In certain embodiments, the VEGF may be added to the culture media at a concentration of 88 ng/mL. In certain embodiments, the VEGF may be added to the culture media at a concentration of 90 ng/mL. In certain embodiments, the VEGF may be added to the culture media at a concentration of 92 ng/mL. In certain embodiments, the VEGF may be added to the culture media at a concentration of 94 ng/mL. In certain embodiments, the VEGF may be added to the culture media at a concentration of 96 ng/mL. In certain embodiments, the VEGF may be added to the culture media at a concentration of 98 ng/mL. In certain embodiments, the VEGF may be added to the culture media at a concentration of 100 ng/mL. In some embodiments of any of the above methods for producing pluripotent stem cell-derived HPCs, vascular endothelial growth factor (VEGF) may be absent from the culture media.

In some embodiments of any of the above methods for producing pluripotent stem cell-derived HPCs, cytokines may be present in the culture media. In some embodiments, cytokines may be absent from the culture media. In some embodiments, any number of cytokines disclosed herein may be present in the culture media. In some embodiments, any number of cytokines disclosed herein may be present in the culture media except for IL-3, which may be absent from the culture media.

In some embodiments of any of the above methods for producing pluripotent stem cell-derived HPCs, the feeder cells may be stromal cells. In some embodiments, the stromal cells may be OP-9 cells. The OP-9 cells may be optionally transduced with a ligand, such as but not limited to a Notch ligand. Non-limiting examples of a Notch ligand are Jagged2, DLL1, DKK3, and DLL4. In some embodiment, the OP-9 cells are OP9-Jagged2, OP9-DLL1, OP9-DLL3, or OP9-DLL4 cells.

In some embodiments of any of the above methods for producing pluripotent stem cell-derived HPCs, the pluripotent stem cells are induced pluripotent stem cells such as, but not limited to induced pluripotent stem cells (iPSCs) or embryonic stem cells (ESCs). In certain embodiments, the pluripotent stem cells are iPSCs. In some embodiments, the iPSCs may be generated from any of various somatic cells. Non-limiting examples of somatic cells may be peripheral blood mononuclear cells (PBMCs), peripheral blood natural killer cells (PBNKs), epithelial cells, fibroblasts, or adipocytes. In some embodiments, the iPSCs may be generated from NK cells. In some embodiments, the iPSCs of the present disclosure are generated by induction of expression of various genes such as but, not limited to, Oct3/4, Sox2, Klf4, c-Myc, NANOG, and Lin28. As a non-limiting example, the iPSCs may be generated by the induction of expression of Oct3/4, Sox2, Klf4, and c-Myc. As another non-limiting example, the iPSCs may be generated by the induction of expression of Oct3/4, NANOG, Sox2, and Lin28.

In some embodiments, the iPSCs may be generated from endothelial cells, hematopoietic progenitor cells (HPCs), lymphoid cells, or myeloid cells.

In some embodiments, the methods of producing HPCs may further comprise a step of purifying HPCs, for example, after maturation. In some embodiments, purification of HPCs may be achieved by isolating CD34+.

### Innate Immune System in the Tube

The innate immune system is a first level line of defense against a variety of pathogens and cancer cells. The cellular components of innate immunity consist, amongst others, of NK cells, macrophages, granulocytes, eosinophils, and endothelial cells. A high level of mutagenicity of certain pathogens (e.g. SARS-CoV-2) and cancer cells emphasize the advantage of cellular immunity over antibody dependent therapies, including vaccines. The cells of innate immune system are developing in the vascular organoid-based differentiation system described herein.

In some embodiments, innate immune system in the tube may collectively refer to any of various cells of the innate immune system such as, but not limited to, cell types of myeloid or lymphoid cell lineage, or combinations thereof, exhibiting any number of immune functions, well known to the skilled artisan, and produced using the vascular organoid-based differentiation system of the present disclosure.

Furthermore, iPSC-derived hematopoietic cells may be developed the way that they will not contain T-cells therefore may be used “off the shelf” as they will not contain T-cells and thus there is no risk for GVHD.

### Natural Killer Cells

In certain aspects is provided natural killer (NK) cells and methods for producing said NK cells from any of the stem cells or pluripotent stem cells disclosed herein to generate e.g., pluripotent stem cell-derived NK cells.

In various embodiments, the method for producing natural killer (NK) cells may comprise: (a) plating the pluripotent stem cells at a seeding density of 1-5×10⁶ cells per 60 mm dish and culturing them overnight to produce colonies of 10-100 cells, b) inducing differentiation of the cells generated in step (a) by incubating said cells for about 2 days in an induction media comprising ascorbic acid and a Wnt activator, c) removing the Wnt activator and continuing incubation for about 3 days, d) detaching cell layers formed in step (c) enzymatically and/or mechanically, and plating them onto matrix-coated dishes or onto a monolayer of feeder cells, e) culturing the cells plated in step (d) for about 12 days in alpha-MEM differentiation media comprising 10% FBS, ascorbic acid, SCF, IL-3, IL-7, IL-15, and FLT3-L, resulting in the formation of a three-dimensional vascular organoid which promotes differentiation of NK cells, f) collecting floating cells formed during step (e) and re-plating them onto matrix-coated dishes or onto a semi-confluent monolayer of feeder cells, g) culturing the replated cells of step (f) for about 10-16 days in maturation media comprising cytokines and the Wnt activator, but not comprising IL-3, to promote NK cell maturation, and h) optionally, purifying NK cells generated in step (g).

In some embodiments, the method for producing NK cells may comprise plating pluripotent stem cells and culturing them in the presence of a growth factor such as, but not limited to, Fibroblast Growth Factor 2 (FGF2). As a non-limiting example, the FGF2 may be present at a concentration of 20-100 ng/mL. In some embodiments, the method for producing NK cells may comprise plating pluripotent stem cells and culturing them in absence of a growth factor.

In some embodiments, the plating of the pluripotent stem cells may be at a seeding density of 1-5×10⁶ cells per 60 mm dish. As a non-limiting example, the plating of the pluripotent stem cells may be at a seeding density of 1×10⁶ cells per 60 mm dish, 2×10⁶ cells per 60 mm dish, 3×10⁶ cells per 60 mm dish, 4×10⁶ cells per 60 mm dish, or 5×10⁶ cells per 60 mm dish. In certain embodiments, the pluripotent stem cells may be cultured for any period of time. As a non-limiting example, the pluripotent stem cells may be cultured overnight. In certain embodiments, the pluripotent stem cells may be cultured for any period of time, e.g., overnight, to produce colonies of cells comprising any number of cells such as, but not limited to, 10-100 cells. As a non-limiting example, the colonies of cells may comprise 10 cells, 12 cells, 14 cells, 16 cells, 18 cells, 20 cells, 22 cells, 24 cells, 26 cells, 28 cells, 30 cells, 32 cells, 34 cells, 36 cells, 38 cells, 40 cells, 42 cells, 44 cells, 46 cells, 48 cells, 50 cells, 52 cells, 54 cells, 56 cells, 58 cells, 60 cells, 62 cells, 64 cells, 66 cells, 68 cells, 70 cells, 72 cells, 74 cells, 76 cells, 78 cells, 80 cells, 82 cells, 84 cells, 86 cells, 88 cells, 90 cells, 92 cells, 94 cells, 96 cells, 98 cells, or 100 cells.

In some embodiments, the colonies of cells may be induced to differentiate by any one of various methods disclosed herein. In some embodiments, the colonies of cells may be induced to differentiate by incubating said cells for any number of days in an induction media. In some embodiments, the colonies of cells may be induced to differentiate by incubating said cells for about 2 days in an induction media. In some embodiments, the induction media may comprise ascorbic acid. In certain embodiments, the ascorbic acid may be present in the induction media at the concentration of 60 μg/mL. In some embodiments the induction media may comprise a Wnt activator. In some embodiments, the induction media may comprise ascorbic acid and a Wnt activator. In certain embodiments, the Wnt activator may be a Wnt protein such as, but not limited to a Wnt4 protein. In certain embodiments, the Wnt activator may target Glycogen synthase kinase-3 (GSK-3). Non-limiting examples of Wnt activators that may target GSK-3 include SB-216763 (Coghlan et al., 2000 [31]), CHIR99021 (PubChem), BIO (6-bromoindirubin-3′-oxime) (Sato et al, 2004 [32]), and LY2090314 (Atkinson et al., 2015 [33]). In certain embodiments, the Wnt activator may target any one of Secreted frizzled-related protein (SFRP), Notum, Protein phosphatase 2 (PP2A), ADP-ribosylation factor GTPase-activating protein 1 (ARFGAP1), and beta-catenin. Non-limiting examples of such Wnt activators may include WAY-316606 (Bodine et al., 2009 [34]), ABC99 (Suciu et al, 2018 [35]), (hetero)arylpyrimidines (Gilbert et al., 2009 [36]), IQ-1 (Miyabayashi et al., 2007 [37]), QS11 (Zhang et al., 2007 [38]), DCA (Pai et al., 2004 [39]), and 2-amino-4-[3,4-(methylenedioxy)benzyl-amino]-6-(3-methoxyphenyl)pyrimidine (Liu et al., 2005 [40]). In some embodiments, the Wnt activator may be removed from the media.

In some embodiments, vascular endothelial growth factor (VEGF) may be added to the culture media. In certain embodiments, VEGF may not be added to the culture media.

In some embodiments, the cells generated using any of the methods described above may be further incubated for a period of time such as, but not limited to, about 3 days to produce hematopoietic progenitor cells. In some embodiments, the hematopoietic progenitor cells may form a cell layer. In some embodiments, the layer of hematopoietic progenitor cells may be detached either enzymatically or mechanically. In some embodiments, the layer of hematopoietic progenitor cells may comprise CD31/CD34/CD144 triple-positive cells that may be optionally purified using conventional methodology by those skilled in the art.

In some embodiments, the hematopoietic cells may be plated onto a matrix-coated dish.

In some embodiments, the matrix coated dish may be coated with, as an example, fibronectin, gelatin or collagen.

In some embodiments, the hematopoietic cells may be plated onto a fibronectin coated dishes. In some embodiments, the hematopoietic cells may be plated onto a monolayer of feeder cells. The monolayer of feeder cells may be confluent. The monolayer of feeder cells may be non-confluent e.g., semi-confluent. The monolayer of feeder cells may be about 60% confluent to overconfluent.

In some embodiments, the plated hematopoietic cells may be cultured for any number of days, e.g., 12 days, in a differentiation media, said differentiation media may comprise alpha-MEM differentiation media comprising e.g., 10% FBS, retinoic acid, MTG, ascorbic acid, SCF, IL-3, IL-7, IL-15, and FLT3-L. In some embodiments, the differentiation media may comprise 10% FBS, ascorbic acid, SCF, IL-3, IL-7, IL-15, and FLT3-L. In some embodiments, the differentiation media does not comprise cytokines and no cytokines are present.

In some embodiments, any of the various method steps of culturing, preparing, and/or purifying any of the cells disclosed herein may exclude cytokines such that no cytokines are present. As non-limiting example, steps (a)-(c) of any of the above disclosed methods may exclude cytokines such that no cytokines are present.

In some embodiments, the culturing of the hematopoietic cells with the differentiation media results in the formation of a three-dimensional organoid. In some embodiments, the three-dimensional vascular organoid may promote differentiation of the hematopoietic cells disclosed herein to form NK cells, e.g., pluripotent stem cell-derived NK cells of the present disclosure. In some embodiments, the cells differentiated using the three-dimensional vascular organoid may be floating cells. In some embodiments, the floating cells differentiated using the three-dimensional vascular organoid may be collected and replated onto a matrix-coated dish. In some embodiments, the floating cells differentiated using the three-dimensional vascular organoid may be collected and replated onto a monolayer of feeder cells which may be confluent. In some embodiments, the floating cells differentiated using the three-dimensional vascular organoid may be collected and replated onto a monolayer of feeder cells which may be non-confluent, e.g. semi-confluent. In some embodiments, the replated floating cells may be cultured for any number of days such as, but not limited to, about 10-16 days. In some embodiments the replated floating cells may be cultured for any number of days in a maturation media. In some embodiments the replated floating cells may be cultured 10-16 days in a maturation media. The maturation media may comprise cytokines such as, but not limited to, SCF, IL-3, IL-7, and/or IL-15. The maturation media may comprise any cytokines of the present disclosure, but may not comprise IL-3. In some embodiments, the maturation media may comprise cytokines and any number of various Wnt activators disclosed herein. In some embodiments, the maturation media may comprise cytokines but not comprise IL-3, and any number of various Wnt activators disclosed herein. As a non-limiting example, the maturation media may comprise SCF, IL-7, IL-15 and a Wnt activator disclosed herein, but may not comprise IL-3. In some embodiments, the maturation media may promote NK cell maturation thereby producing NK cells derived from pluripotent stem cells, i.e., pluripotent stem cell-derived NK cells.

In some embodiments of any of the above methods for producing pluripotent stem cell-derived NK cells, the FGF2 may be present in the induction media at the concentration of 20-100 ng/mL. As a non-limiting example, the FGF2 may be present in the induction media at a concentration of 20 ng/mL, 22 ng/mL, 24 ng/mL, 26 ng/mL, 28 ng/mL, 30 ng/mL, 32 ng/mL, 34 ng/mL, 36 ng/mL, 38 ng/mL, 40 ng/mL, 42 ng/mL, 44 ng/mL, 46 ng/mL, 48 ng/mL, 50 ng/mL, 52 ng/mL, 54 ng/mL, 56 ng/mL, 58 ng/mL, 60 ng/mL, 62 ng/mL, 64 ng/mL, 66 ng/mL, 68 ng/mL, 70 ng/mL, 72 ng/mL, 74 ng/mL, 76 ng/mL, 78 ng/mL, 80 ng/mL, 82 ng/mL, 84 ng/mL, 86 ng/mL, 88 ng/mL, 90 ng/mL, 92 ng/mL, 94 ng/mL, 96 ng/mL, 98 ng/mL, or 100 ng/mL.

In some embodiments of any of the above methods for producing pluripotent stem cell-derived NK cells, the ascorbic acid may be present in the induction media at the concentration of 20-100 μg/mL. As a non-limiting example, the ascorbic acid may be present in the induction media at a concentration of 20 μg/mL, 22 μg/mL, 24 μg/mL, 26 μg/mL, 28 μg/mL, 30 μg/mL, 32 μg/mL, 34 μg/mL, 36 μg/mL, 38 μg/mL, 40 μg/mL, 42 μg/mL, 44 μg/mL, 46 μg/mL, 48 μg/mL, 50 μg/mL, 52 μg/mL, 54 μg/mL, 56 μg/mL, 58 μg/mL, 60 μg/mL, 62 μg/mL, 64 μg/mL, 66 μg/mL, 68 μg/mL, 70 μg/mL, 72 μg/mL, 74 μg/mL, 76 μg/mL, 78 μg/mL, 80 μg/mL, 82 μg/mL, 84 μg/mL, 86 μg/mL, 88 μg/mL, 90 μg/mL, 92 μg/mL, 94 μg/mL, 96 μg/mL, 98 μg/mL, or 100 μg/mL.

In some embodiments of any of the above methods for producing pluripotent stem cell-derived NK cells, the ascorbic acid may be present in the induction media at the concentration of 60 μg/mL.

In some embodiments of any of the above methods for producing pluripotent stem cell-derived NK cells, the Wnt activator may be CHIR99021 (CAS registry number 252917-06-9). In some embodiments, the CHIR99021 may be present in the induction media disclosed herein. In some embodiments, the CHIR99021 may be absent from the induction media disclosed herein. In some embodiments, the CHIR99021 may present in the induction media at a concentration of about 3-8 μM. As a non-limiting example, the CHIR99021 may be present in the induction media at a concentration of about 3 μM, 4 μM, 5 μM, 6 μM, 7 μM, or 8 μM.

In some embodiments of any of the above methods for producing pluripotent stem cell-derived NK cells, vascular endothelial growth factor (VEGF) may be added to the culture media. As a non-limiting example, VEGF may be added to the culture media at a concentration of 20-100 ng/mL. In certain embodiments, the VEGF may be added to the culture media at a concentration of 20 ng/mL. In certain embodiments, the VEGF may be added to the culture media at a concentration of 22 ng/mL. In certain embodiments, the VEGF may be added to the culture media at a concentration of 24 ng/mL. In certain embodiments, the VEGF may be added to the culture media at a concentration of 26 ng/mL. In certain embodiments, the VEGF may be added to the culture media at a concentration of 28 ng/mL. In certain embodiments, the VEGF may be added to the culture media at a concentration of 30 ng/mL. In certain embodiments, the VEGF may be added to the culture media at a concentration of 32 ng/mL. In certain embodiments, the VEGF may be added to the culture media at a concentration of 34 ng/mL. In certain embodiments, the VEGF may be added to the culture media at a concentration of 36 ng/mL. In certain embodiments, the VEGF may be added to the culture media at a concentration of 38 ng/mL. In certain embodiments, the VEGF may be added to the culture media at a concentration of 40 ng/mL. In certain embodiments, the VEGF may be added to the culture media at a concentration of 42 ng/mL. In certain embodiments, the VEGF may be added to the culture media at a concentration of 44 ng/mL. In certain embodiments, the VEGF may be added to the culture media at a concentration of 46 ng/mL. In certain embodiments, the VEGF may be added to the culture media at a concentration of 48 ng/mL. In certain embodiments, the VEGF may be added to the culture media at a concentration of 50 ng/mL. In certain embodiments, the VEGF may be added to the culture media at a concentration of 52 ng/mL. In certain embodiments, the VEGF may be added to the culture media at a concentration of 54 ng/mL. In certain embodiments, the VEGF may be added to the culture media at a concentration of 56 ng/mL. In certain embodiments, the VEGF may be added to the culture media at a concentration of 58 ng/mL. In certain embodiments, the VEGF may be added to the culture media at a concentration of 60 ng/mL. In certain embodiments, the VEGF may be added to the culture media at a concentration of 62 ng/mL. In certain embodiments, the VEGF may be added to the culture media at a concentration of 64 ng/mL. In certain embodiments, the VEGF may be added to the culture media at a concentration of 66 ng/mL. In certain embodiments, the VEGF may be added to the culture media at a concentration of 68 ng/mL. In certain embodiments, the VEGF may be added to the culture media at a concentration of 70 ng/mL. In certain embodiments, the VEGF may be added to the culture media at a concentration of 72 ng/mL. In certain embodiments, the VEGF may be added to the culture media at a concentration of 74 ng/mL. In certain embodiments, the VEGF may be added to the culture media at a concentration of 76 ng/mL. In certain embodiments, the VEGF may be added to the culture media at a concentration of 78 ng/mL. In certain embodiments, the VEGF may be added to the culture media at a concentration of 80 ng/mL. In certain embodiments, the VEGF may be added to the culture media at a concentration of 82 ng/mL. In certain embodiments, the VEGF may be added to the culture media at a concentration of 84 ng/mL. In certain embodiments, the VEGF may be added to the culture media at a concentration of 86 ng/mL. In certain embodiments, the VEGF may be added to the culture media at a concentration of 88 ng/mL. In certain embodiments, the VEGF may be added to the culture media at a concentration of 90 ng/mL. In certain embodiments, the VEGF may be added to the culture media at a concentration of 92 ng/mL. In certain embodiments, the VEGF may be added to the culture media at a concentration of 94 ng/mL. In certain embodiments, the VEGF may be added to the culture media at a concentration of 96 ng/mL. In certain embodiments, the VEGF may be added to the culture media at a concentration of 98 ng/mL. In certain embodiments, the VEGF may be added to the culture media at a concentration of 100 ng/mL. In some embodiments of any of the above methods for producing pluripotent stem cell-derived NK cells, vascular endothelial growth factor (VEGF) may be absent from the culture media.

In some embodiments of any of the above methods for producing pluripotent stem cell-derived NK cells, cytokines may be present in the culture media. Non-limiting examples of cytokines are IL-3, IL-7, and IL-1. In some embodiments, cytokines may be absent from the culture media. In some embodiments, any number of cytokines disclosed herein may be present in the culture media. In some embodiments, any number of cytokines disclosed herein may be present in the culture media except for IL-3, which may be absent from the culture media.

In some embodiments of any of the above methods for producing pluripotent stem cell-derived NK cells, the feeder cells may be stromal cells. In some embodiments, the stromal cells may be OP-9 cells. The OP-9 cells may be optionally transduced with a ligand, such as but not limited to a Notch ligand. Non-limiting examples of a Notch ligand are Jagged2, DLL1, DKK3, and DLL4. In some embodiment, the OP-9 cells are OP9-Jagged2, OP9-DLL1, OP9-DLL3, or OP9-DLL4 cells.

In some embodiments of any of the above methods for producing pluripotent stem cell-derived NK cells, the pluripotent stem cells are induced pluripotent stem cells such as, but not limited to induced pluripotent stem cells (iPSCs) or embryonic stem cells (ESCs). In certain embodiments, the pluripotent stem cells are iPSCs. In some embodiments, the iPSCs may be generated from any of various somatic cells. Non-limiting examples of somatic cells may be peripheral blood mononuclear cells (PBMCs), peripheral blood natural killer cells (PBNKs), epithelial cells, fibroblasts, or adipocytes. In some embodiments, the iPSCs may be generated from NK cells. In some embodiments, the iPSCs of the present disclosure are generated by induction of expression of various genes such as but, not limited to, Oct3/4, Sox2, Klf4, c-Myc, NANOG, and Lin28. As a non-limiting example, the iPSCs may be generated by the induction of expression of Oct3/4, Sox2, Klf4, and c-Myc. As another non-limiting example, the iPSCs may be generated by the induction of expression of Oct3/4, NANOG, Sox2, and Lin28.

In some embodiments, the iPSCs may be generated from endothelial cells, hematopoietic progenitor cells (HPCs), lymphoid cells, or myeloid cells.

The pluripotent stem cell-derived NK cells produced using the methods of the present disclosure may be expanded by culturing them in expansion media comprising IL-2. In some embodiments, the expansion media may comprise 50-300 U (units) IL-2. As a non-limiting example, the expansion media may comprise IL-2 at a unit of 50 U, 55 U, 60 U, 65 U, 70 U, 75 U, 80 U, 85 U, 90 U, 95 U, 100 U, 105 U, 110 U, 115 U, 120 U, 125 U, 130 U, 135 U, 140 U, 145 U, 150 U, 155 U, 160 U, 165 U, 170 U, 175 U, 180 U, 185 U, 190 U, 195 U, 200 U, 205 U, 210 U, 215 U, 220 U, 225 U, 230 U, 235 U, 240 U, 245 U, 250 U, 255 U, 260 U, 265 U, 270 U, 275 U, 280 U, 285 U, 290 U, 295 U, or 300 U. In some embodiments, the expansion media disclosed herein may be RPMI-1640. In some embodiments, the expansion media disclosed herein may be RPMI-1640 supplemented with 5-10% FBS. In some embodiments, the expansion media disclosed herein may be RPMI-1640. In some embodiments, the expansion media disclosed herein may be RPMI-1640 supplemented with 1% FBS. In some embodiments, the expansion media disclosed herein may be RPMI-1640. In some embodiments, the expansion media disclosed herein may be RPMI-1640 supplemented with 2% FBS. In some embodiments, the expansion media disclosed herein may be RPMI-1640. In some embodiments, the expansion media disclosed herein may be RPMI-1640 supplemented with 3% FBS. In some embodiments, the expansion media disclosed herein may be RPMI-1640. In some embodiments, the expansion media disclosed herein may be RPMI-1640 supplemented with 4% FBS. In some embodiments, the expansion media disclosed herein may be RPMI-1640. In some embodiments, the expansion media disclosed herein may be RPMI-1640 supplemented with 5% FBS. In some embodiments, the expansion media disclosed herein may be RPMI-1640. In some embodiments, the expansion media disclosed herein may be RPMI-1640 supplemented with 6% FBS. In some embodiments, the expansion media disclosed herein may be RPMI-1640. In some embodiments, the expansion media disclosed herein may be RPMI-1640 supplemented with 7% FBS. In some embodiments, the expansion media disclosed herein may be RPMI-1640. In some embodiments, the expansion media disclosed herein may be RPMI-1640 supplemented with 8% FBS. In some embodiments, the expansion media disclosed herein may be RPMI-1640. In some embodiments, the expansion media disclosed herein may be RPMI-1640 supplemented with 9% FBS. In some embodiments, the expansion media disclosed herein may be RPMI-1640. In some embodiments, the expansion media disclosed herein may be RPMI-1640 supplemented with 10% FBS.

In some embodiments, the pluripotent stem cell-derived NK cells produced using the methods of the present disclosure may be expanded by culturing them in the presence of allogenic feeder cells. As a non-limiting example, the allogenic feeder cells may be cancer cells. In some embodiments, the cancer cells may be K562 cells.

In some embodiments, the allogenic feeder cells may comprise plasma membrane particles derived from the cancer cells. In some embodiments, the plasma membrane particles may be derived from the K562 cells. In some embodiments, the plasma membrane particles may be e.g., IL-15, IL-21 or 41BBL, or variants or combinations thereof. The IL-15, IL21 or 41BBL, or variants or combinations thereof, may be derived from K562 cells. As a non-limiting example, K562 cells comprising plasma membrane particles may be K562-mb15-41BBL cells. In some embodiments, the K562-mb15-41BBL may comprise IL-15 and 4BBL. The K562-mb15-41BBL and their plasma membrane particles, e.g., plasma membrane particles derived from the K562-mb15-41BBL, may be prepared using conventional methodology by those skilled in the art, such as in [50]. As a non-limiting example, the K562-mb15-41BBL cells are grown in RPMI-1640 media supplemented with 5% FBS. The K562-mb15-41BBL cells may be harvested by centrifugation (e.g., 1000×g, 10 minutes) and washed with Dulbecco's PBS containing 2 mM EDTA. The K562-mb15-41BBL cells may then be resuspended in lysis buffer containing 50 mM HEPES, pH 7.4, 150 mM NaCl, 2 mM MgCl₂ and AEBSF, aprotinin, leupeptin, and pepstatin A. The K562-mb15-41BBL cells are then disrupted by nitrogen cavitation e.g., at 300 psi for 30 minutes at 4° C. The K562-mb15-41BBL cells lysate may be centrifuged (e.g., 1000×g, 10 minutes) and the supernatant then centrifuged (e.g., 100,000×g) to pellet the crude cell membranes. The crude membranes may be further purified by sucrose gradient centrifugation, and the fraction that corresponds to closed plasma membrane particles (or vesicles) may be collected [50].

Non-limiting examples of plasma membrane particles derived from cancer cells may be IL-15, IL-21 and 41BBL. As a non-limiting example, the allogenic feeder cells may be K562 cells. In some embodiments, the K562 may be irradiated. In some embodiments, the K562 cells may express IL-15. In some embodiments, the K562 cells may express IL-21. In some embodiments, K562 cells may express 41BBL.

The pluripotent stem cell-derived NK cells produced using the methods of the present disclosure may be developmentally young “rejuvenated” cells which may proliferate better and stay longer in the patient body compared to modified “adult” NK cells in accordance with principles described by Goya and colleagues [41] which is incorporated herein by reference in its entirety.

In some embodiments, the methods of producing NK cells may further comprise a step of purifying NK cells, for example, after maturation. In some embodiments, purification of NK cells may be achieved by isolating CD56+ cells.

### Myeloid Cells

In certain aspects is provided myeloid cells and methods for producing said myeloid cells from any of the stem cells or pluripotent stem cells disclosed herein to generate, e.g., pluripotent stem cell-derived myeloid cells. Myeloid cells are a morphologically, phenotypically, and functionally distinct cell types that include granulocytes (neutrophils, eosinophils, and basophils), monocytes, macrophages, erythrocytes, megakaryocytes, and mast cells.

In various embodiments, the method for producing myeloid cells may comprise: a) plating the pluripotent stem cells at a seeding density of 1-5×10⁶ cells per 60 mm dish and culturing them overnight to produce colonies of 10-100 cells, b) inducing differentiation of the cells generated in step (a) by incubating said cells for about 2 days in an induction media comprising ascorbic acid and a Wnt activator, c) removing the Wnt activator and continuing incubation for about 3 days to produce, d) detaching cell layers formed in step (c) enzymatically and/or mechanically, and plating them onto matrix-coated dishes or onto a monolayer of feeder cells, e) culturing the cells plated in step (d) for about 12 days in alpha-MEM differentiation media comprising 10% FBS, ascorbic acid, along with other cytokines that may be specific to a particular myeloid lineage, thereby resulting in the formation of a three-dimensional vascular organoid which promotes differentiation myeloid cells, f) collecting floating cells formed during step (e) and re-plating them onto matrix-coated dishes or onto a semi-confluent monolayer of feeder cells, g) culturing the replated cells of step (f) for about 7-14 days in maturation media comprising cytokines to promote myeloid cell maturation, and h) optionally, purifying the myeloid cells generated in step (g).

Cytokines used in step (d) of the method for producing myeloid cells described herein can include those that are specific to a particular myeloid lineage, for instance, monocytes and macrophages as described, for example, in Brok-Volchanskaya et al., 2019 [60], Cao et al., 2019 [61], and U.S. Patent Publication No. US 2020/0385676, each of which are hereby incorporated by reference herein in their entirety for all purposes; megakaryocytes, as described, for example, in Kammers et al., 2021 [63], which is hereby incorporated by reference herein in its entirety for all purposes; myeloid and NK progenitors as described, for example, in Mesquitta et al., 2019 [64], which is hereby incorporated by reference herein in its entirety for all purposes; T-cells, as described, for example, in Iriguchi et al, 2021 [62], which is hereby incorporated by reference herein in its entirety for all purposes; and neutrophils, as described, for example, in Majumder et al., 2020 [76] and U.S. Patent Publication No. US 2020/0385676, both of which are hereby incorporated by reference herein in their entirety for all purposes.

In some embodiments, the method for producing myeloid cells may comprise plating pluripotent stem cells and culturing them in the presence of a growth factor such as, but not limited to, Fibroblast Growth Factor 2 (FGF2). As a non-limiting example, the FGF2 may be present at a concentration of 20-100 ng/mL. In some embodiments, the method for producing myeloid cells may comprise plating pluripotent stem cells and culturing them in absence of a growth factor.

In some embodiments, the plating of the pluripotent stem cells may be at a seeding density of 1-5×10⁶ cells per 60 mm dish. As a non-limiting example, the plating of the pluripotent stem cells may be at a seeding density of 1×10⁶ cells per 60 mm dish, 2×10⁶ cells per 60 mm dish, 3×10⁶ cells per 60 mm dish, 4×10⁶ cells per 60 mm dish, or 5×10⁶ cells per 60 mm dish. In certain embodiments, the pluripotent stem cells may be cultured for any period of time. As a non-limiting example, the pluripotent stem cells may be cultured overnight. In certain embodiments, the pluripotent stem cells may be cultured for any period of time, e.g., overnight, to produce colonies of cells comprising any number of cells such as, but not limited to, 10-100 cells. As a non-limiting example, the colonies of cells may comprise 10 cells, 12 cells, 14 cells, 16 cells, 18 cells, 20 cells, 22 cells, 24 cells, 26 cells, 28 cells, 30 cells, 32 cells, 34 cells, 36 cells, 38 cells, 40 cells, 42 cells, 44 cells, 46 cells, 48 cells, 50 cells, 52 cells, 54 cells, 56 cells, 58 cells, 60 cells, 62 cells, 64 cells, 66 cells, 68 cells, 70 cells, 72 cells, 74 cells, 76 cells, 78 cells, 80 cells, 82 cells, 84 cells, 86 cells, 88 cells, 90 cells, 92 cells, 94 cells, 96 cells, 98 cells, or 100 cells.

In some embodiments, the colonies of cells may be induced to differentiate by any one of various methods disclosed herein. In some embodiments, the colonies of cells may be induced to differentiate by incubating said cells for any number of days in an induction media. In some embodiments, the colonies of cells may be induced to differentiate by incubating said cells for about 2 days in an induction media. In some embodiments, the induction media may comprise ascorbic acid. In certain embodiments, the ascorbic acid may be present in the induction media at the concentration of 60 μg/mL. In some embodiments the induction media may comprise a Wnt activator. In some embodiments, the induction media may comprise ascorbic acid and a Wnt activator. In certain embodiments, the Wnt activator may be a Wnt protein such as, but not limited to a Wnt4 protein. In certain embodiments, the Wnt activator may target Glycogen synthase kinase-3 (GSK-3). Non-limiting examples of Wnt activators that may target GSK-3 include SB-216763 (Coghlan et al., 2000 [31]), CHIR99021 (PubChem), BIO (6-bromoindirubin-3′-oxime) (Sato et al, 2004 [32]), and LY2090314 (Atkinson et al., 2015 [33]). In certain embodiments, the Wnt activator may target any one of Secreted frizzled-related protein (SFRP), Notum, Protein phosphatase 2 (PP2A), ADP-ribosylation factor GTPase-activating protein 1 (ARFGAP1), and beta-catenin. Non-limiting examples of such Wnt activators may include WAY-316606 (Bodine et al., 2009 [34]), ABC99 (Suciu et al, 2018 [35]), (hetero)arylpyrimidines (Gilbert et al., 2009 [36]), IQ-1 (Miyabayashi et al., 2007 [37]), QS11 (Zhang et al., 2007 [38]), DCA (Pai et al., 2004 [39]), and 2-amino-4-[3,4-(methylenedioxy)benzyl-amino]-6-(3-methoxyphenyl)pyrimidine (Liu et al., 2005 [40]). In some embodiments, the Wnt activator may be removed from the media.

In some embodiments, vascular endothelial growth factor (VEGF) may be added to the culture media. In certain embodiments, VEGF may not be added to the culture media.

In some embodiments, the cells generated using any of the methods described above may be further incubated for a period of time such as, but not limited to, about 3 days to produce, for example, hematopoietic progenitor cells. In some embodiments, the hematopoietic progenitor cells may comprise a cell layer. In some embodiments, the cell layer may be detached either enzymatically or mechanically. In some embodiments, the layer of hematopoietic progenitor cells may comprise CD31/CD34/CD144 triple-positive cells that may be optionally purified using conventional methodology by those skilled in the art.

In some embodiments, the hematopoietic cells may be plated onto a matrix-coated dish.

In some embodiments, the matrix coated dish may be coated with, as an example, fibronectin, gelatin or collagen.

In some embodiments, the hematopoietic cells may be plated onto a fibronectin coated dishes. In some embodiments, the hematopoietic cells may be plated onto a monolayer of feeder cells. The monolayer of feeder cells may be confluent. The monolayer of feeder cells may be non-confluent e.g., semi-confluent. The monolayer of feeder cells may be about 60% confluent to overconfluent.

In some embodiments, the plated hematopoietic cells may be cultured for any number of days, e.g., 12 days, in a differentiation media, said differentiation media may comprise alpha-MEM differentiation media comprising e.g., 10% FBS, ascorbic acid, cytokines specific to a particular myeloid lineage, thereby resulting in the formation of a three-dimensional vascular organoid which may promote the differentiation of myeloid cells. In some embodiments, the differentiation media may comprise 10% FBS, ascorbic acid, cytokines specific to a particular myeloid lineage. In some embodiments, the differentiation media does not comprise cytokines and no cytokines are present.

In some embodiments, any of the various method steps of culturing, preparing, and/or purifying any of the cells disclosed herein may exclude cytokines such that no cytokines are present. As non-limiting example, steps (a)-(c) of any of the above disclosed methods may exclude cytokines such that no cytokines are present.

In some embodiments, the culturing of the hematopoietic cells with the differentiation media results in the formation of a three-dimensional organoid. In some embodiments, the three-dimensional vascular organoid may promote differentiation of the hematopoietic cells disclosed herein to form myeloid cells, e.g., pluripotent stem cell-derived myeloid cells of the present disclosure. In some embodiments, the floating cells differentiated using the three-dimensional vascular organoid may be collected and replated onto a matrix-coated dish. In some embodiments, the cells differentiated using the three-dimensional vascular organoid may be floating cells. In some embodiments, the floating cells differentiated using the three-dimensional vascular organoid may be collected and replated onto a monolayer of feeder cells which may be confluent. In some embodiments, the floating cells differentiated using the three-dimensional vascular organoid may be collected and replated onto a monolayer of feeder cells which may be non-confluent, e.g. semi-confluent. In some embodiments, the replated floating cells may be cultured for any number of days such as, but not limited to, about 7-14 days. In some embodiments the replated floating cells may be cultured for any number of days in a maturation media. In some embodiments the replated floating cells may be cultured 7-14 days in a maturation media. The maturation media may comprise cytokines such as, but not limited to, cytokines specific to a particular myeloid lineage. The maturation media may comprise cytokines such as, but not limited to, SCF, IL-7, and/or IL-15. In some embodiments, the maturation media may promote myeloid cell maturation thereby producing myeloid cells derived from pluripotent stem cells, i.e., pluripotent stem cell-derived myeloid cells.

In some embodiments of any of the above methods for producing pluripotent stem cell-derived myeloid, the FGF2 may be present in the induction media at the concentration of 20-100 ng/mL. As a non-limiting example, the FGF2 may be present in the induction media at a concentration of 20 ng/mL, 22 ng/mL, 24 ng/mL, 26 ng/mL, 28 ng/mL, 30 ng/mL, 32 ng/mL, 34 ng/mL, 36 ng/mL, 38 ng/mL, 40 ng/mL, 42 ng/mL, 44 ng/mL, 46 ng/mL, 48 ng/mL, 50 ng/mL, 52 ng/mL, 54 ng/mL, 56 ng/mL, 58 ng/mL, 60 ng/mL, 62 ng/mL, 64 ng/mL, 66 ng/mL, 68 ng/mL, 70 ng/mL, 72 ng/mL, 74 ng/mL, 76 ng/mL, 78 ng/mL, 80 ng/mL, 82 ng/mL, 84 ng/mL, 86 ng/mL, 88 ng/mL, 90 ng/mL, 92 ng/mL, 94 ng/mL, 96 ng/mL, 98 ng/mL, or 100 ng/mL.

In some embodiments of any of the above methods for producing pluripotent stem cell-derived myeloid cells, the ascorbic acid may be present in the induction media at the concentration of 20-100 μg/mL. As a non-limiting example, the ascorbic acid may be present in the induction media at a concentration of 20 μg/mL, 22 μg/mL, 24 μg/mL, 26 μg/mL, 28 μg/mL, 30 μg/mL, 32 μg/mL, 34 μg/mL, 36 μg/mL, 38 μg/mL, 40 μg/mL, 42 μg/mL, 44 μg/mL, 46 μg/mL, 48 μg/mL, 50 μg/mL, 52 μg/mL, 54 μg/mL, 56 μg/mL, 58 μg/mL, 60 μg/mL, 62 μg/mL, 64 μg/mL, 66 μg/mL, 68 μg/mL, 70 μg/mL, 72 μg/mL, 74 μg/mL, 76 μg/mL, 78 μg/mL, 80 μg/mL, 82 μg/mL, 84 μg/mL, 86 μg/mL, 88 μg/mL, 90 μg/mL, 92 μg/mL, 94 μg/mL, 96 μg/mL, 98 μg/mL, or 100 μg/mL.

In some embodiments of any of the above methods for producing pluripotent stem cell-derived myeloid cells, the ascorbic acid may be present in the induction media at the concentration of 60 μg/mL.

In some embodiments of any of the above methods for producing pluripotent stem cell-derived myeloid cells, the Wnt activator may be CHIR99021 (CAS registry number 252917-06-9). In some embodiments, the CHIR99021 may be present in the induction media disclosed herein. In some embodiments, the CHIR99021 may be absent from the induction media disclosed herein. In some embodiments, the CHIR99021 may present in the induction media at a concentration of about 3-8 μM. As a non-limiting example, the CHIR99021 may be present in the induction media at a concentration of about 3 μM, 4 μM, 5 μM, 6 μM, 7 μM, or 8 μM. In some embodiments, the CHIR99021 may be present in the indication mediate at a concentration of 6 μM.

In some embodiments of any of the above methods for producing pluripotent stem cell-derived myeloid cells, vascular endothelial growth factor (VEGF) may be added to the culture media. As a non-limiting example, VEGF may be added to the culture media at a concentration of 20-100 ng/mL. In certain embodiments, the VEGF may be added to the culture media at a concentration of 20 ng/mL. In certain embodiments, the VEGF may be added to the culture media at a concentration of 22 ng/mL. In certain embodiments, the VEGF may be added to the culture media at a concentration of 24 ng/mL. In certain embodiments, the VEGF may be added to the culture media at a concentration of 26 ng/mL. In certain embodiments, the VEGF may be added to the culture media at a concentration of 28 ng/mL. In certain embodiments, the VEGF may be added to the culture media at a concentration of 30 ng/mL. In certain embodiments, the VEGF may be added to the culture media at a concentration of 32 ng/mL. In certain embodiments, the VEGF may be added to the culture media at a concentration of 34 ng/mL. In certain embodiments, the VEGF may be added to the culture media at a concentration of 36 ng/mL. In certain embodiments, the VEGF may be added to the culture media at a concentration of 38 ng/mL. In certain embodiments, the VEGF may be added to the culture media at a concentration of 40 ng/mL. In certain embodiments, the VEGF may be added to the culture media at a concentration of 42 ng/mL. In certain embodiments, the VEGF may be added to the culture media at a concentration of 44 ng/mL. In certain embodiments, the VEGF may be added to the culture media at a concentration of 46 ng/mL. In certain embodiments, the VEGF may be added to the culture media at a concentration of 48 ng/mL. In certain embodiments, the VEGF may be added to the culture media at a concentration of 50 ng/mL. In certain embodiments, the VEGF may be added to the culture media at a concentration of 52 ng/mL. In certain embodiments, the VEGF may be added to the culture media at a concentration of 54 ng/mL. In certain embodiments, the VEGF may be added to the culture media at a concentration of 56 ng/mL. In certain embodiments, the VEGF may be added to the culture media at a concentration of 58 ng/mL. In certain embodiments, the VEGF may be added to the culture media at a concentration of 60 ng/mL. In certain embodiments, the VEGF may be added to the culture media at a concentration of 62 ng/mL. In certain embodiments, the VEGF may be added to the culture media at a concentration of 64 ng/mL. In certain embodiments, the VEGF may be added to the culture media at a concentration of 66 ng/mL. In certain embodiments, the VEGF may be added to the culture media at a concentration of 68 ng/mL. In certain embodiments, the VEGF may be added to the culture media at a concentration of 70 ng/mL. In certain embodiments, the VEGF may be added to the culture media at a concentration of 72 ng/mL. In certain embodiments, the VEGF may be added to the culture media at a concentration of 74 ng/mL. In certain embodiments, the VEGF may be added to the culture media at a concentration of 76 ng/mL. In certain embodiments, the VEGF may be added to the culture media at a concentration of 78 ng/mL. In certain embodiments, the VEGF may be added to the culture media at a concentration of 80 ng/mL. In certain embodiments, the VEGF may be added to the culture media at a concentration of 82 ng/mL. In certain embodiments, the VEGF may be added to the culture media at a concentration of 84 ng/mL. In certain embodiments, the VEGF may be added to the culture media at a concentration of 86 ng/mL. In certain embodiments, the VEGF may be added to the culture media at a concentration of 88 ng/mL. In certain embodiments, the VEGF may be added to the culture media at a concentration of 90 ng/mL. In certain embodiments, the VEGF may be added to the culture media at a concentration of 92 ng/mL. In certain embodiments, the VEGF may be added to the culture media at a concentration of 94 ng/mL. In certain embodiments, the VEGF may be added to the culture media at a concentration of 96 ng/mL. In certain embodiments, the VEGF may be added to the culture media at a concentration of 98 ng/mL. In certain embodiments, the VEGF may be added to the culture media at a concentration of 100 ng/mL. In some embodiments of any of the above methods for producing pluripotent stem cell-derived myeloid cells, vascular endothelial growth factor (VEGF) may be absent from the culture media.

In some embodiments of any of the above methods for producing pluripotent stem cell-derived myeloid cells, cytokines may be present in the culture media. In some embodiments, cytokines may be absent from the culture media. In some embodiments, any number of cytokines disclosed herein may be present in the culture media. In some embodiments, any number of cytokines disclosed herein may be present in the culture media except for IL-3, which may be absent from the culture media.

In some embodiments of any of the above methods for producing pluripotent stem cell-derived myeloid cells, the feeder cells may be stromal cells. In some embodiments, the stromal cells may be OP-9 cells. The OP-9 cells may be optionally transduced with a ligand, such as but not limited to a Notch ligand. Non-limiting examples of a Notch ligand are Jagged2, DLL1, DKK3, and DLL4. In some embodiment, the OP-9 cells are OP9-Jagged2, OP9-DLL1, OP9-DLL3, or OP9-DLL4 cells.

In some embodiments of any of the above methods for producing pluripotent stem cell-derived myeloid cells, the pluripotent stem cells are induced pluripotent stem cells such as, but not limited to induced pluripotent stem cells (iPSCs) or embryonic stem cells (ESCs). In certain embodiments, the pluripotent stem cells are iPSCs. In some embodiments, the iPSCs may be generated from any of various somatic cells. Non-limiting examples of somatic cells may be peripheral blood mononuclear cells (PBMCs), peripheral blood natural killer cells (PBNKs), epithelial cells, fibroblasts, or adipocytes. In some embodiments, the iPSCs may be generated from NK cells. In some embodiments, the iPSCs of the present disclosure are generated by induction of expression of various genes such as but, not limited to, Oct3/4, Sox2, Klf4, c-Myc, NANOG, and Lin28. As a non-limiting example, the iPSCs may be generated by the induction of expression of Oct3/4, Sox2, Klf4, and c-Myc. As another non-limiting example, the iPSCs may be generated by the induction of expression of Oct3/4, NANOG, Sox2, and Lin28.

In some embodiments, the iPSCs may be generated from endothelial cells, hematopoietic progenitor cells (HPCs), lymphoid cells, or myeloid cells.

In some embodiments, the methods of producing myeloid cells may further comprise a step of purifying myeloid cells, for example, after maturation. In some embodiments, purification of myeloid cells may be achieved by isolating CD15+, CD14+, CD11b+, CD33+, and/or CD235+ cells.

In some embodiments, the purifying of the myeloid cells may be achieved by isolating CD15+ cells. In some embodiments, the purifying of the myeloid cells may be achieved by isolating CD14+ cells. In some embodiments, the purifying of the myeloid cells may be achieved by isolating CD11b+ cells. In some embodiments, the purifying of the myeloid cells may be achieved by isolating CD33+ cells. In some embodiments, the purifying of the myeloid cells may be achieved by isolating CD235+ cells.

### Modification of Cells

In certain aspects is provided modified cells and methods of modifying the cells produced using the methods described herein.

In one aspect, provided herein is modified NK cells such that they lack expression of an NK inhibitory molecule or manifest a reduced expression of an NK inhibitory molecule. In some embodiments, the NK cells are modified such that they modulate expression of an NK inhibitory molecule or inhibit the expression of an NK inhibitory molecule. In some embodiments, the modified NK cells provided herein may include a population of cells comprising NK cells which have been modified to express one or more NK inhibitory molecules at a lower level than NK cells that are not modified with respect to expression levels of the NK inhibitory molecules. In certain aspects, the NK inhibitory molecule which is expressed at a modulated, reduced, or null level is NKG2A.

In some embodiments, the NK inhibitory molecule that is modulated or is reduced in expression in the population of cells comprising NK cells is NKG2A. In some embodiments, the NKG2A expression has been knocked out. As a non-limiting example, the NKG2A expression has been knocked out by Clustered Regularly Interspaced Short Palindromic Repeats (CRISPR)-Cas nuclease, e.g. a CRISPR/Cas9 nuclease, a zinc finger nuclease (ZFN), a transcription activator-like effector nuclease TALEN nuclease, or a meganuclease. In some embodiments, the NKG2A expression has been knocked down. As a non-limiting example, the NKG2A expression has been knocked down by an RNA interference (RNAi)-related technique. As a non-limiting example, the RNAi-related technique may be a short hairpin RNA (shRNA). A short hairpin RNA or small hairpin RNA (shRNA/Hairpin Vector) is an artificial RNA molecule with a tight hairpin turn that may be used to silence target gene expression via RNAi. The shRNAs may be incorporated into plasmid vectors and integrated into genomic DNA for long-term or stable expression, for extended knockdown of the target mRNA.

In some embodiments, the NK inhibitory molecule that is modulated or is reduced in expression in the population of cells comprising NK cells is NKG2A. In some embodiments, the NKG2A expression has been knocked out. In some embodiments, the NKG2A expression has been knocked out by a Clustered Regularly Interspaced Short Palindromic Repeats (CRISPR)-Cas nuclease, e.g. a CRISPR/Cas9 nuclease, a zinc finger nuclease (ZFN), a transcription activator-like effector nuclease TALEN nuclease, or a meganuclease. In some embodiments, the NKG2A expression has been knocked out by a CRISPR/Cas nuclease. In some embodiments, the NKG2A expression has been knocked out by a CRISPR/Cas9 nuclease. In some embodiments, the knockout of NKG2A expression in the NK cells generates a population of cells comprising NK cells having a higher cytotoxicity against tumor cells than NK cells in which NKG2A has not been knocked out, such as naturally occurring NK cells. In some embodiments, the tumor cells are multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, and retinoblastoma cells. In some embodiments, the tumor cells are solid tumor cells. In some alternatives, the solid tumor cells are liver tumor cells, lung tumor cells, pancreatic tumor cells, renal tumor cells, or glioblastoma multiforme (GBM) cells.

In some embodiments, the NK inhibitory molecule that is modulated or is reduced in expression in the population of cells comprising NK cells is NKG2A. In some embodiments, the NKG2A expression is knocked down. In some embodiments, the NKG2A expression may be knocked down by an RNA interference (RNAi)-related technique. The active components of RNAi are short/small double stranded RNAs (dsRNAs) called small interfering RNAs (siRNAs). As a non-limiting example, the RNAi-related technique may comprise siRNA molecules, e.g. siRNA duplexes, targeting a NKG2A, which may be designed and synthesized in vitro and introduced into cells to activate RNAi. According to the present disclosure, one or more siRNA molecules that target NKG2A are designed. In some embodiments, one or more siRNA molecules used in the methods described herein comprise the nucleotide sequence of any one of SEQ ID NO: 5-8. In some embodiments, one or more siRNA molecules used in the methods described herein consist of the nucleotide sequence of any one of SEQ ID NO: 5-8. In some embodiments, one or more siRNA molecules used in the methods described herein target a nucleotide sequence comprising any one of SEQ ID NO: 9-10. In some embodiments, one or more siRNA molecules used in the methods described herein target a nucleotide consisting of any one of SEQ ID NO: 9-10. In some embodiments, the knockdown of NKG2A expression in the NK cells generates a population of cells comprising NK cells having a higher cytotoxicity against tumor cells than NK cells in which NKG2A has not been knocked down, such as naturally occurring NK cells. In some embodiments, the tumor cells are selected from the group consisting of multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, and retinoblastoma cells. In some embodiments, the tumor cells are solid tumor cells. In some embodiments, the solid tumor cells are selected from the group consisting of liver tumor cells, lung tumor cells, pancreatic tumor cells, renal tumor cells, and glioblastoma multiforme (GBM) cells.

In some embodiments, the modified NK cells comprising a modulated, reduced, or null level is NKG2A may be useful for the induction or lysis or killing of senescent cells. In some embodiments, the modified NK cells comprising a modulated, reduced, or null level is NKG2A may be useful for the induction or lysis or killing of cancer cells. In some embodiments, the senescent cells are mammalian senescent cells. In some embodiments, the cancer cells are mammalian cancer cells.

Any of the foregoing molecules, including molecules comprising CRISPR/Cas nuclease and/or RNAi molecules e.g., siRNAs, may be delivered by a vector. In various embodiments, the vector is a viral or non-viral vector. In some embodiments, the vector is a viral vector is a retroviral vector, a lentiviral vector, an adenoviral vector, an adeno-associated viral (AAV) vector, an alphaviral vector, vaccinia virus vector, a herpes simplex virus vector, or a baculoviral vector. In some embodiments, the vector is a lentiviral vector. In some embodiments, the vector is an adeno-associated viral (AAV) vector. In some embodiments, the vector is a non-viral vector.

In certain aspects is provided modified NK cells. In some embodiments, the modified NK cells are modified using targeted gene editing achieved with the use of one or more of a gene editing nuclease. In some embodiments, the gene editing nuclease is a Clustered Regularly Interspaced Short Palindromic Repeats (CRISPR)/Cas nuclease, a zinc finger nuclease (ZFN), a transcription activator-like effector nuclease (TALEN), or a meganuclease.

In some embodiments, gene editing nucleases used in the methods of the present disclosure include one or more CRISPR/Cas nucleases. CRISPR/Cas nucleases are RNA-guided nucleases derived from the acquired immunity system (known as the CRISPR/Cas system) found in bacteria and archaea. See, e.g., U.S. Pat. No. 8,697,359, which is incorporated by reference in its entirety for all purposes. The CRISPR loci is a region within the organism's genome where short segments of foreign DNA are integrated between short repeat palindromic sequences. These loci are transcribed into long RNA transcripts (“pre-crRNA”) which are further processed into short CRISPR RNAs (crRNAs). There are two classes of CRISPR/Cas systems which all incorporate these RNAs and proteins known as CRISPR-associated (Cas) proteins.

In some embodiments, gene editing nucleases used in the methods of the present disclosure include one or more Cas9 molecules. Cas9 is found in class 2 CRISPR/Cas systems and specifically Type II CRISPR/Cas systems. In Type II systems, a trans-activating RNA (tracrRNA) complementary to repeat sequences in the pre-crRNA, triggers the processing of a crRNA by a double strand-specific RNase III in the presence of the Cas9 protein. Cas9 is then complexed with the crRNA and tracrRNA to form a ribonucleoprotein (RNP) which is able to cleave a target DNA that is complementary to a spacer-derived sequence in the mature crRNA. The cleavage by Cas9 is also dependent on the presence of a short motif in the target DNA referred to as the proto-spacer adjacent motif (PAM) sequence (see Qi et al (2013) Cell 152:1173, which is incorporated herein by reference in its entirety). The tracrRNA is also required for targeting as its base pairs with the crRNA at its 3′ end, and this structure triggers Cas9 activity. The Cas9 protein induces a double-strand DNA break in the target DNA using its two nuclease domains, an HNH endonuclease domain and a Ruv endonuclease domain, each responsible for cleaving the complementary DNA strand and the non-complementary DNA strand, respectively.

Alternatively, the requirement of the crRNA-tracrRNA complex can be circumvented by use of a chimeric “single guide RNA” (sgRNA) that comprises the hairpin normally formed by the annealing of the crRNA and the tracrRNA (see Jinek et al (2012) Science 337:816 and Cong et al (2013) Sciencexpress/10.1126/science.1231143, which are incorporated herein by reference in their entirety).

Cas9 molecules of a variety of species, or variants thereof, can be used in the methods and compositions described herein. For example, Cas9 molecules used in the present invention may be derived from e.g., S. pyogenes, S. thermophilus, Staphylococcus aureus and/or Neisseria meningitidis Cas9 molecules. Additional Cas9 species include but are not limited to, Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhiz obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae. In some embodiments, the Cas9 protein is a Streptococcus pyogenes Cas9 protein or Staphylococcus aureus Cas9 protein.

In some embodiments, the Cas9 protein is a wild-type Cas9 protein. Other useful Cas9 proteins include a Cas9 nickase, a dead Cas9 (dCas9), or a split Cas9.

In some embodiments, gene editing nucleases used in the methods of the present disclosure comprise one or more Cas12a molecules. Cas12a (also known as Cpf1) is another RNA-guided endonuclease of the class 2 CRISPR/Cas systems. As a gene editing nuclease, Cas12a has a few advantages over Cas9, for example, it has a smaller size and it requires only one crRNA molecule. The Cpf1-crRNA complex cleaves a target DNA that is complementary to the targeting domain in the crRNA and introduces a sticky-end-like DNA double-strand break of 4 or 5 nucleotides overhang.

Non-limiting examples of other Cas gene editing nucleases which can be used in the methods of the present invention include, e.g., CasX, CasY, C2C2, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966.

Other gene editing nucleases such as zinc finger nucleases (ZFNs), transcription activator-like effector nucleases (TALENs), or meganucleases can also be used to introduce site-specific DNA breaks. See, for example, Urnov et al. (2010) Nature 435(7042):646-51; U.S. Pat. Nos. 8,586,526; 6,534,261; 6,599,692; 6,503,717; 6,689,558; 7,067,317; 7,262,054, each of which are incorporated by reference in their entirety for all purposes.

The introduction of site-specific single (SSBs) or double strand breaks (DSBs) allows for target sequence alteration through, for example, non-homologous end-joining (NHEJ) or homology-directed repair (HDR). In the absence of a template nucleic acid, a cell with a cleaved genome will resort to the error prone NHEJ pathway to repair the break. This process often adds or deletes nucleotides during the repair process (“indels”) which may lead to the introduction of missense or non-sense mutations at the target site. This can result in knocking out (e.g., complete lack of transcription or altered transcription) of the target gene of interest.

Gene editing can also include the knocking in of genes in addition to the knockout methods described above. In order for an exogenous nucleic acid to be inserted into a target locus, a template nucleic acid molecule will be provided which contains homology arms directed to the target gene locus. The cell will use the template nucleic acid to repair the cleaved target DNA sequence via homology-directed repair (HDR), resulting in the transfer of genetic information from the template nucleic acid to the target DNA.

Gene editing nucleases can be engineered to target one or more target sites having sequence complementary to the guide RNA including. Targeting may occur within or near a target site described herein (e.g., about 10 base pairs (bp), 20 bp, 50 bp, 100 bp, 200 bp, 500 bp or less than 1000 bp either 5′ or 3′ to the target site).

When CRISPR/Cas nucleases are used as the gene editing nucleases, one or more guide RNAs (gRNAs) are provided, which can direct the CRISPR/Cas nucleases to a target DNA sequence having complementarity to the targeting domain in the gRNA. In some embodiments, one or more guide RNAs (gRNAs) used in the methods described herein comprise a targeting domain comprising the nucleotide sequence of any one of SEQ ID NO: 1-4. In some embodiments, one or more guide RNAs (gRNAs) used in the methods described herein comprise a targeting domain consisting of the nucleotide sequence of any one of SEQ ID NO: 1-4.

In some aspects, the modified NK cells disclosed herein may be engineered to express a chimeric antigen receptor (CAR) or an antigen-specific TCR. In some embodiments, methods described herein include the step of introducing into cells an exogenous nucleic acid molecule comprising a nucleotide sequence coding for a CAR or an antigen-specific TCR.

The exogenous nucleic acid molecule comprising a nucleotide sequence coding for a CAR or an antigen-specific TCR may be episomally expressed. Alternatively, the exogenous nucleic acid molecule comprising a nucleotide sequence coding for a CAR or an antigen-specific TCR may be knocked into the locus of an HLA class I gene or HLA class II gene via homology directed repair (HDR). For example, and not by limitation, the exogenous nucleic acid molecule comprising a nucleotide sequence coding for a CAR or an antigen-specific TCR may be knocked into a B2M, RFX5, RFXANK, or RFXAP locus to replace the endogenous gene. In the case of knock-in, the nucleic acid molecule comprising a nucleotide sequence coding for a CAR or an antigen-specific TCR may be provided as a double stranded DNA (dsDNA), a single-stranded DNA (ssDNA), or in a viral vector (e.g., AAV). In either embodiments, the gene is operatively linked (i.e.: under transcriptional control) to a promoter active in the cells.

The CAR or the antigen-specific TCR may be directed against an antigen expressed at the surface of a malignant or infected cell, such as a tumor antigen or an infectious antigen.

Non-limiting examples of tumor antigens that may be targeted by the modified cells described herein include human epidermal growth factor receptor 2 (HER2), interleukin-13 receptor subunit alpha-2 (IL-13Ra2), ephrin type-A receptor 2 (EphA2), A kinase anchor protein 4 (AKAP-4), adrenoceptor beta 3 (ADRB3), anaplastic lymphoma kinase (ALK), immunoglobulin lambda-like polypeptide 1 (IGLL1), androgen receptor, angiopoietin-binding cell surface receptor 2 (Tie 2), B7H3 (CD276), bone marrow stromal cell antigen 2 (BST2), carbonic anhydrase IX (CAIX), CCCTC-binding factor (Zinc Finger Protein)-like (BORIS), CD171, CD179a, CD24, CD300 molecule-like family member f (CD300LF), CD38, CD44v6, CD72, CD79a, CD79b, CD97, chromosome X open reading frame 61 (CXORF61), claudin 6 (CLDN6), CS-1 (CD2 subset 1, CRACC, SLAMF7, CD319, or 19A24), C-type lectin domain family 12 member A (CLECi2A), C-type lectin-like molecule-1 (CLL-1), Cyclin B 1, Cytochrome P450 1B 1 (CYP1B 1), EGF-like module-containing mucin-like hormone receptor-like 2 (EMR2), epidermal growth factor receptor (EGFR), ERG (transmembrane protease, serine 2 (TMPRSS2) ETS fusion gene), ETS translocation-variant gene 6, located on chromosome 12p (ETV6-AML), Fc fragment of IgA receptor (FCAR), Fc receptor-like 5 (FCRL5), Fms-like tyrosine kinase 3 (FLT3), Folate receptor beta, Fos-related antigen 1, Fucosyl GM1, G protein-coupled receptor 20 (GPR20), G protein-coupled receptor class C group 5, member D (GPRC5D), ganglioside GD3, ganglioside GM3, glycoceramide (GloboH), Glypican-3 (GPC3), Hepatitis A virus cellular receptor 1 (HAVCR1), hexasaccharide portion of globoH, high molecular weight-melanoma-associated antigen (HMWMAA), human Telomerase reverse transcriptase (hTERT), interleukin 11 receptor alpha (IL-11Ra), KIT (CD117), leukocyte-associated immunoglobulin-like receptor 1 (LAIR1), leukocyte immuno-globulin-like receptor subfamily A member 2 (LILRA2), Lewis (Y) antigen, lymphocyte antigen 6 complex, locus K 9 (LY6K), lymphocyte antigen 75 (LY75), lymphocyte-specific protein tyro-sine kinase (LCK), mammary gland differentiation antigen (NY-BR-1), melanoma cancer testis antigen-1 (MAD-CT-1), melanoma cancer testis antigen-2 (MAD-CT-2), melanoma inhibitor of apoptosis (ML-IAP), mucin 1, cell surface associated (MUC1), N-acetyl glucosaminyl-transferase V (NA17), neural cell adhesion molecule (NCAM), o-acetyl-GD2 ganglioside (OAcGD2), olfactory receptor 51E2 (OR51E2), p53 mutant, paired box protein Pax-3 (PAX3), paired box protein Pax-5 (PAX5), pannexin 3 (PANX3), placenta-specific 1 (PLAC1), platelet-derived growth factor receptor beta (PDGFR-beta), Polysialic acid, proacrosin binding protein sp32 (OY-TES 1), prostate stem cell antigen (PSCA), Protease Serine 21 (PRSS21), Proteasome (Prosome, Macro-pain) Subunit, Beta Type, 9 (LMP2), Ras Homolog Family Member C (RhoC), sarcoma translocation breakpoints, sialyl Lewis adhesion molecule (sLe), sperm protein 17 (SPA17), squamous cell carcinoma antigen recognized by T cells 3 (SART3), stage-specific embryonic antigen-4 (SSEA-4), synovial sarcoma, X breakpoint 2 (SSX2), TCR gamma alternate reading frame protein (TARP), TGS5, thyroid stimulating hormone receptor (TSHR), Tn antigen (Tn Ag), tumor endothelial marker 1 (TEM1/CD248), tumor endothelial marker 7-related (TEM7R), uroplakin 2 (UPK2), vascular endothelial growth factor receptor 2 (VEGFR2), v-myc avian myelocytomatosis viral oncogene neuroblastoma derived homolog (MYCN), Wilms tumor protein (WTi), and X Antigen Family, Member 1A (XAGEl), or a fragment or variant thereof.

Additional antigens that may be targeted by the modified cells described herein include, but are not limited to, carbonic anhydrase EX, alpha-fetoprotein, A3, antigen specific for A33 antibody, Ba 733, BrE3-antigen, CA125, CD1, CDla, CD3, CD5, CD15, CD16, CD19, CD20, CD21, CD22, CD23, CD25, CD30, CD33, CD38, CD45, CD74, CD79a, CD80, CD138, colon-specific antigen-p (CSAp), CEA (CEACAM5), CEACAM6, CSAp, EGFR, EGP-I, EGP-2, Ep-CAM, EphA1, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC1, MUC2, MUC3, MUC4, NCA66, NCA95, NCA90, antigen specific for PAM-4 anti-body, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

An infectious antigen may be a viral antigen, a bacterial antigen, a fungal antigen, a parasite antigen, or a prion antigen, or the like. Infectious antigens include the intact microorganism (e.g., virus, bacterium, fungus) as well as natural isolates and fragments or derivatives thereof and also synthetic or recombinant compounds which are identical to or similar to natural microorganism antigens and induce an immune response specific for that microorganism (e.g., virus, bacterium, fungus). A compound is similar to a natural microorganism antigen if it induces an immune response (humoral and/or cellular) to a natural microorganism antigen. Such antigens are used routinely in the art and are well known to the skilled artisan.

An infectious antigen may be an infectious virus or derived from an infectious virus. Non-limiting examples of infectious viruses that have been found in humans include but are not limited to: Adenoviridae (most adenoviruses); Arena viridae (hemorrhagic fever viruses); Birnaviridae; Bungaviridae (e.g., Hantaan viruses, bunga viruses, phleboviruses and Nairo viruses); Calciviridae (e.g., strains that cause gastroenteritis); Coronoviridae (e.g., coronaviruses); Filoviridae (e.g., ebola viruses); Flaviridae (e.g., dengue viruses, encephalitis viruses, yellow fever viruses); Hepadnaviridae (Hepatitis B virus); Herpesviridae (herpes simplex virus (HSV) 1 and 2, varicella zoster virus, cytomegalovirus (CMV), herpes virus); Iridoviridae (e.g., African swine fever virus); Norwalk and related viruses, and astroviruses; Orthomyxoviridae (e.g., influenza viruses); Papovaviridae (papilloma viruses, polyoma viruses); Paramyxoviridae (e.g., parainfluenza viruses, mumps virus, measles virus, respiratory syncytial virus); Parvovirida (parvoviruses); Picornaviridae (e.g., polio viruses, hepatitis A virus; enteroviruses, human Coxsackie viruses, rhinoviruses, echoviruses); Poxviridae (variola viruses, vaccinia viruses, pox viruses); Reoviridae (e.g., reoviruses, orbiviurses and rotaviruses); Retroviridae (e.g., human immunodeficiency viruses, such as HIV-1 (also referred to as HTLV-III, LAV or HTLV-III/LAV, or HIV-III); and other isolates, such as HIV-LP); Rhabdoviridae (e.g., vesicular stomatitis viruses, rabies viruses); Togaviridae (e.g., equine encephalitis viruses, rubella viruses); and unclassified viruses (e.g., the etiological agents of Spongiform encephalopathies, the agent of delta hepatitis, the agents of non-A, non-B hepatitis (i.e. Hepatitis C).

An infectious antigen may be an infectious bacterium or derived from an infectious bacterium. Both gram negative and gram positive bacteria can serve as antigens in vertebrate animals. Such gram positive bacteria include, but are not limited to, Pasteurella species, Staphylococci species and Streptococcus species. Grain negative bacteria include, but are not limited to, Escherichia coli, Pseudomonas species, and Salmonella species. Non-limiting examples of infectious bacteria include but are not limited to: Actinomyces israelli, Bacillus antracis, Bacteroides sp., Borelia burgdorferi, Chlamydia., Clostridium perfringers, Clostridium tetani, Corynebacterium diphtheriae, Corynebacterium sp., Enterobacter aerogenes, Enterococcus sp., Erysipelothrix rhusiopathiae, Fusobacterium nucleatum, Haemophilus influenzae, Helicobacter pyloris, Klebsiella pneumoniae, Legionella pneumophilia, Leptospira, Listeria monocytogenes, Mycobacteria sps. (e.g., M tuberculosis, M avium, M gordonae, M intracellulare, M kansaii), Neisseria gonorrhoeae, Neisseria meningitidis, Pasteurella multocida, pathogenic Campylobacter sp., Rickettsia, Staphylococcus aureus, Streptobacillus monihformis, Streptococcus (anaerobic sps.), Streptococcus (viridans group), Streptococcus agalactiae (Group B Streptococcus), Streptococcus bovis, Streptococcus faecalis, Streptococcus pneumoniae, Streptococcus pyogenes (Group A Streptococcus), Treponema pallidium, and Treponema pertenue.

An infectious antigen may be or derived from other infectious microorganisms. Non-limiting examples of infectious fungi include: Cryptococcus neoformans, Histoplasma capsulatuin, Coccidioides immitis, Blastomyces dernatitidis, Chlamydia trachomatis and Candida albicans. Other infectious organisms (i.e., protists) include: Plasmodium such as Plasmodium falciparum, Plasmodium malariae, Plasmodium ovale, Plasmodium vivax, Toxoplasma gondii and Shistosoma. Other medically relevant microorganisms have been descried extensively in the literature, e.g., see C. G. A. Thomas, “Medical Microbiology”, Bailliere Tindall, Great Britain 1983, which is hereby incorporated by reference in its entirety.

Other non-limiting examples of infectious antigens include viral antigens such as HIV antigens (e.g., gp120, gp160, p18, Tat, Gag, Pol, Env, Nef), glycoprotein from Herpesvirus, and surface antigen and core antigen from Hepatitis B virus; bacterial antigens such as OspA, OspB and OspC antigens from Borrelia sp; fungal and parasite antigens such as MP65 from Candida albicans and CS protein from Plasmodium sp.

Various embodiments of the methods described above involves introducing into cells one or more polynucleotide/polypeptide agents (e.g., gene editing nucleases, guide RNAs, RNAi molecules, CARs). The polynucleotides and/or polypeptides described in the present invention may be introduced into the cell via viral, non-viral gene delivery methods, or a physical method. Suitable methods for polynucleotide and/or polypeptide delivery for use the methods of the present invention include any method known by those of skill in the art, by which a polynucleotide and/or polypeptide can be introduced into an organelle, cell, tissue, or organism. The polynucleotide and/or polypeptide transfer may be carried out in vitro, ex vivo, or in vivo.

In various embodiment, polypeptides or polynucleotides are introduced into cells using a physical method. Suitable physical methods include, but are not limited to, electroporation, direct injection (e.g., microinjection), magnetofection, ultrasound, a ballistic or hydrodynamic method, or a combination thereof.

Electroporation is a method for polynucleotide and/or polypeptide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In some embodiments, cell wall-degrading enzymes, such as pectin-degrading enzymes, can be employed to render the cells more susceptible to genetic modification by electroporation than untreated cells. See e.g., U.S. Pat. No. 5,384,253, incorporated herein by reference in its entirety for all purposes.

As a non-limiting example, one or more CRISPR/Cas nucleases and one or more gRNAs may be assembled to form one or more ribonucleoprotein (RNP) complexes which are then introduced into the cells by electroporation.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference in their entirety for all purposes.

Another physical method for polynucleotide and/or polypeptide transfer includes injection. In some embodiments, a polypeptide, a polynucleotide, or a vector may be delivered to a cell, tissue, or organism via one or more injections (e.g., a needle injection). Non-limiting methods of injection include injection of a composition (e.g., a saline based composition). Polynucleotides and/or polynucleotides can also be introduced by direct microinjection. Non-limiting sites of injection include, subcutaneous, intradermal, intramuscular, intranodal (allows for direct delivery of antigen to lymphoid tissues), intravenous, intraprostatic, intratumor, intralymphatic (allows direct administration of dendritic cells) and intraperitoneal. It is understood that proper site of injection preparation is necessary (e.g., shaving of the site of injection to observe proper needle placement).

In some embodiments, polynucleotides and/or polypeptides described in the present invention are introduced into cells by pinocytosis induced by hypertonicity or hypotonicity. For example, the cells maybe placed into a buffer that has either a higher or lower salt concentration than normal saline. This may activate an active uptake mechanism in the cells where they engulf the extracellular environment. Various chemicals can be used to enhance and modify this process.

It may not require any special machinery. Exemplary ways that pinocytosis can be used for transduction are described in the art. See e.g., WO2017093326A1, which is hereby incorporated by reference in its entirety for all purposes.

In various embodiments, polynucleotides and/or polypeptides described in the present invention are introduced into cells via a vector. The vector may be a viral vector or a non-viral vector.

In some embodiments, the vector is a viral vector. Suitable viral vectors that can be used in the present invention include, but are not limited to, a retroviral vector, a lentiviral vector, an adeno-viral vector, an adeno-associated viral (AAV) vector, an alphaviral vector, vaccinia virus vector, a herpes simplex virus vector, or a baculoviral vector. In one specific embodiment, the viral vector is a lentiviral vector. In one specific embodiment, the viral vector is a retroviral vector. In some embodiments, cells are transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety.

In some embodiments, the vector is a non-viral vector. Non-limiting examples of non-viral vectors useful in the methods of the present invention include a plasmid or a transposon.

Nucleic acid vaccines may also be used to transfer polynucleotides into the cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In some embodiments, polynucleotides and/or polypeptides may be introduced into the cells using a nanoparticle, a polymer, a dendrimer, a liposome, and a polyethylenimine (PEI) particle. In some embodiments, polypeptides (e.g., CRISPR/Cas nucleases) are introduced into the cells as a soluble protein or ribonucleoprotein.

Additional methods of polynucleotide and/or polypeptide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide and/or polypeptide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and/or polypeptide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

It should be recognized that in the case of CRISPR/Cas nucleases, the Cas protein (e.g., Cas9, Cas12a) and the gRNA need not to be delivered using the same method. In some embodiments, the Cas protein (e.g., Cas9, Cas12a) and the gRNA are delivered using the same method. For example, both the Cas protein (e.g., Cas9, Cas12a) and the gRNA can be introduced into the cells via electroporation or in the same vector. In some embodiments, the Cas protein (e.g., Cas9, Cas12a) and the gRNA are delivered using different methods. For example, the Cas protein (e.g., Cas9, Cas12a) is introduced into the cells via electroporation and the gRNA is delivered in viral vector. As another example, the Cas protein (e.g., Cas9, Cas12a) and the gRNA are delivered in separate vectors.

### Therapeutic Methods

In one aspect, the present disclosure provides methods for treating diseases or disorders in a subject in need thereof with any of the HPCs, NK cell(s), and/or myeloid cells of the present disclosure, or the pharmaceutical composition(s) comprising any of the HPCs, NK cell(s), and/or myeloid cells described herein.

In one aspect is provided a method of treating a disease or disorder in a subject in need thereof comprising administering to the subject a therapeutically effective number of HPCs of the present disclosure or the pharmaceutical composition comprising HPCs of the present disclosure. In some embodiments, the disease of the disorder may be rescue therapy for a patient with cancer after high doses of chemotherapy and radiation as well as the correction of severe deficiencies in the hematopoietic system, or an adoptive immune therapy for malignancies and autoimmune disorders.

In one aspect is provided a method of treating a disease or disorder in a subject in need thereof comprising administering to the subject a therapeutically effective number of myeloid cells of the present disclosure or the pharmaceutical composition comprising myeloid cells of the present disclosure. In some embodiments, the disease of the disorder may be an infection, a cancer, an autoimmune disease, or neutropenia or non-malignant blood disorders.

In one aspect, the present disclosure provides a method for killing a tumor or cancer cell comprising contacting the cell with any of the HPCs, NK cell(s), and/or myeloid cells of the present disclosure, or the pharmaceutical composition(s) comprising any of the HPCs, NK cell(s), and/or myeloid cells described herein.

In one aspect, the present disclosure provides a method for treating a tumor in a subject in need thereof. The method comprises administering to the subject a therapeutically effective amount of the HPCs, NK cell(s), and/or myeloid cells described herein or the pharmaceutical composition.

In various embodiments, the cancer is a solid tumor, a brain tumor, or a hematologic malignancy. In certain embodiments, the hematologic malignancy is AML, ALL, B-ALL, T-ALL, or lymphoma. Examples of tumors are, but not limited to, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., Ewing sarcoma and other Ewing sarcoma family of tumors, osteosarcoma, or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of tumors can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In various embodiments, the tumor is selected from osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma. In various embodiments, the tumor is a solid tumor. In various embodiments, the solid tumor is Ewing's sarcoma, lung adenocarcinoma, osteosarcoma, breast cancer, or prostate cancer. In certain embodiments, the tumor is a brain tumor. In some embodiments, the brain tumor is glioblastoma or neuroblastoma.

In another aspect, the present disclosure provides a method for eliminating a senescent cell comprising contacting the cell with any of the NK cell(s) of the present disclosure, or the pharmaceutical composition(s) described herein. In one aspect, the present disclosure provides a method for treating aging or age-related diseases and/or disorders in a subject in need thereof. The method comprises administering to the subject a therapeutically effective amount of the NK cell(s) described herein or the pharmaceutical composition.

In yet another aspect, the present disclosure provides a method for killing a pathogen-infected cell comprising contacting the cell with any of the NK cell(s) of the present disclosure, or the pharmaceutical composition(s) described herein. In some embodiments, the pathogen infection may be a bacterial infection. In some embodiments, the pathogen infection may be a viral infection. A non-limiting example of a virus-infected cell may be a SARs-CoV2-infected cell. In some embodiments, the present disclosure provides a method for treating SARs-CoV2 infection in a subject in need thereof.

In some embodiments, the present disclosure provides a method for treating SARs-CoV2 infection in a subject in need thereof. In some embodiments, the present disclosure provides a method for treating SARs-CoV2 infection in a subject that is a cancer patient. In some embodiments, the present disclosure provides a method for treating SARs-CoV2 infection and cancer in a subject in need thereof.

In some embodiments, the HPCs, NK cells, and/or myeloid cells as described herein or the pharmaceutical composition as described herein may be useful for treating cancer selected from squamous cell cancer, adenosquamous cell carcinoma, lung cancer, cancer of the peritoneum, hepatocellular cancer, gastric or stomach cancer, cervical cancer, ovarian cancer, liver cancer, bladder cancer, cancer of the urinary tract, hepatoma, breast cancer, colon cancer, colorectal cancer, endometrial cancer, salivary gland carcinoma, kidney or renal cancer, prostate cancer, vulval cancer, thyroid cancer, hepatic carcinoma, anal carcinoma, penile carcinoma, skin cancer, multiple myeloma and acute lymphocytic leukemia (ALL), acute myelocytic leukemia (AML), chronic myelocytic leukemia (CML), and chronic lymphocytic leukemia (CLL), lymphoma such as Hodgkin lymphoma (HL) and non-Hodgkin lymphoma (NHL), follicular lymphoma, chronic lymphocytic leukemia/small lymphocytic lymphoma (CLL/SLL), mantle cell lymphoma (MCL), marginal zone B-cell lymphomas, primary mediastinal B-cell lymphoma, Burkitt lymphoma, lymphoplasmacytic lymphoma, immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL), angioimmunoblastic T-cell lymphoma, extra-nodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, brain, as well as head and neck cancer, biliary cancer, bronchus cancer, chordoma, choriocarcinoma, epithelial carcinoma, endothelial sarcoma, esophageal cancer, Ewing sarcoma, heavy chain disease, hematopoietic cancer, immunocytic amyloidosis, monoclonal gammopathy of undetermined significance, myelodysplastic syndromes, myeloproliferative disorder, agnogenic myeloid metaplasia (AMM) or myelofibrosis (MF), chronic idiopathic myelofibrosis, myeloproliferative neoplasms, polycythemia vera, rectum adenocarcinoma, essential thrombocytosis, chronic neutrophilic leukemia, hypereosinophilic syndrome, or soft tissue sarcoma, and metastases thereof.

In some embodiments, the present disclosure provides a method of treating a disease or disorder in a subject in need thereof, the method including administering to the subject an effective amount of the HPCs, NK cells and/or myeloid cells as described herein or the pharmaceutical composition as described herein.

Diseases or disorders that can be treated using the methods and/or compositions of the present disclosure include, but are not limited to, a cancer, an autoimmune disease, an infection, e.g., an infectious disease, neutropenia, a non-malignant blood disorder, an inherited genetic disorder, a metabolic disorder, a degenerative disorder, or an injury causing permanent tissue damage.

In various embodiments, the disease that may be treated using the methods and/or compositions, e.g., HPCs or pharmaceutical compositions comprising such HPCs, of the present disclosure may be a malignant disease. Examples of malignant disease are, without limitation, multiple myeloma, Hodgkin and non-Hodgkin lymphoma, acute myeloid leukemia, acute lymphocytic leukemia, myelodysplastic syndrome, chronic myeloid leukemia, chronic lymphocytic leukemia, myelofibrosis, essential thrombocytosis, and polycythemia vera. in some embodiments, the malignant disease may be a solid tumor. In some embodiments, the solid tumor may be a germ cell tumor such as, but not limited to, a testicular tumor. In some embodiments, the germ cell tumor may be refractory to chemotherapy, for example, after the third recurrence with chemotherapy. In some embodiments, any of the methods and/or compositions disclosed herein may be useful for the treatment of medulloblastoma, metastatic breast cancer, and other solid tumors.

In various embodiments, the disease that may be treated using the methods and/or compositions of the present disclosure may be a non-malignant disease. Non-limiting examples of non-malignant disease are aplastic anemia, severe combined immune deficiency syndrome (SCID), thalassemia, and sickle cell anemia. Additional non-limiting examples of non-malignant diseases include chronic granulomatous disease, leukocyte adhesion deficiency, Chediak-Higashi syndrome, Kostman syndrome, Fanconi anemia, Blackfan-Diamond anemia, and enzymatic disorders. In some embodiments, any of the methods and/or compositions disclosed herein may be useful for the treatment of autoimmune diseases such as, but not limited to, systemic sclerosis, systemic lupus erythematosus. In some embodiments, any of the methods and/or compositions disclosed herein may be useful for the treatment of relapsing-remitting multiple sclerosis [66].

In some embodiments, the NK cells as described herein or the pharmaceutical composition as described herein may be useful for the treatment of myocardial infarction/ischemia.

In some embodiments, the NK cells as described herein or the pharmaceutical composition as described herein may be useful for the treatment of liver cirrhosis.

In some embodiments, the NK cells as described herein or the pharmaceutical composition as described herein may be useful for the treatment of infectious disease selected from diseases caused by coronaviruses (e.g., diseases caused by SARS-CoV, SARS-CoV2, MERS), HIV, influenza, Herpes, viral hepatitis, Epstein Bar virus, polio, viral encephalitis, measles, chicken pox, Papilloma virus, cytomegalovirus, Rabies, Varicella, Yellow fever, West Nile virus, Ebola, pneumonia, tuberculosis, syphilis, Lyme disease, babesiosis, malaria, trypanosomiasis, leishmaniasis, trichomoniasis, or amoebiasis.

In some embodiments, the NK cells of the present disclosure may be useful for treating a disease or disorder selected from multiple sclerosis, type I and type II diabetes, Parkinson's disease, Alzheimer's disease, amyotrophic lateral sclerosis, heart disease, chronic obstructive pulmonary disease, osteoarthritis, degenerative disc disease, hemoglobinopathies, mucopolysaccharidoses, mucolipidoses, osteopetrosis, Diamond-Blackfan syndrome, or an inborn error of metabolism.

Neutropenia, including febrile neutropenia (FN), represents a major complication associated with cancer chemotherapy, resulting in considerable morbidity, mortality, and costs [65]. The complexity of donor granulocyte collection hampers the effectiveness of granulocyte transfusion for correcting neutropenia. In some embodiments, cells of innate immune system developed in the vascular organoid differentiation system described herein may be useful for the treatment of neutropenia, such as but not limited to FN. In some embodiments, the HPCs, NK cell(s), and/or myeloid cells described herein or the pharmaceutical composition as described herein may be useful for the treatment of neutropenia such as, but not limited to, febrile neutropenia (FN).

In some embodiments, myeloid cells disclosed herein or the pharmaceutical compositions comprising such cells may be useful for treating a disease or disorder that may be specific to a myeloid lineage. Examples of such cells, without limitation, include platelets, red blood cells, macrophages (M1) and macrophages (M2). In some embodiments, the macrophages (M1) may be pro-inflammatory. In some embodiments, the macrophages (M2) may be anti-inflammatory. As a non-limiting example, platelets prepared in accordance with the disclosure and/or their pharmaceutical compositions may be useful for the treatment of thrombocytopenia. In some embodiments, red blood cells prepared in accordance with the disclosure, and/or their pharmaceutical compositions, may be useful for blood transfusions for rare blood types, e.g., AB-negative, B-negative, AB-positive, A-negative, and the like. In some embodiments, red blood cells may be useful for blood transfusions for allo-immunized patients. In some embodiments, when the Macrophages (M1) are pro-inflammatory, such cells prepared in accordance with the disclosure, and/or their pharmaceutical compositions, may be useful for the treatment of tumor growth and/or metastases. In some embodiments, when the Macrophages (M2) are anti-inflammatory, such cells prepared in accordance with the disclosure, and/or their pharmaceutical compositions, may be useful for the treatment of, for example, atherosclerosis. Without wishing to be bound by theory, macrophages e.g., M2 anti-inflammatory macrophages, may be useful for any treatments within the knowledge of one skilled in the art that may benefit from tissue repair.

In some embodiments of any of the therapeutic methods described above, the composition is administered in a therapeutically effective amount. The dosages of the composition administered in the methods of the invention will vary widely, depending upon the subject's physical parameters, the frequency of administration, the manner of administration, the clearance rate, and the like. The initial dose may be larger and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of modified host cells. It is also contemplated that a variety of doses will be effective to improve in vivo effector function of modified host cells.

In some embodiments, composition comprising the HPCs, NK cells and/or myeloid cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of modified host cells will depend on the therapeutic use for which the composition is intended for.

HPCs, NK cells and/or myeloid cells of the present disclosure may be administered multiple times at dosages listed above. The modified host cells may be allogeneic, syngeneic, xenogeneic, or autologous to the patient undergoing therapy.

In various embodiments of the methods described herein, the subject is a human. The subject may be a juvenile, a pediatric subject, or an adult, of any age or sex. In some embodiments, the subject is under the age of 18. In various embodiments, the subject is less than about 3 months, about 6 months, about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, or about 18 years of age. In some embodiments, the subject is about 19 years, about 20 years, about 25 years, about 30 years, about 35 years, about 40 years, abo 45 years, about 50 years, about 55 years, about 60 years, about 65 years, about 70 years, about 75 years, about 80 years, about 85 years, about 90 years, about 95 years, or about 100 years old.

In one aspect the present disclosure also provides a pharmaceutical composition comprising any of the cells of the present disclosure, e.g. HPCs, NK cells and/or myeloid cells, modified cells and optionally a pharmaceutically acceptable carrier and/or excipient. Examples of pharmaceutical carriers include but are not limited to sterile liquids, such as water and oils, including those of petroleum, animal, vegetable, or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising any of the cells described herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising any of the cells described herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In some embodiments, compositions are formulated for parenteral administration, e.g., intravascular (intravenous or intraarterial), intraperitoneal, intratumoral, intraventricular, intrapleural or intramuscular administration. In some embodiments, the composition is reconstituted from a lyophilized preparation prior to administration.

In some embodiments, the modified cells may be mixed with substances that adhere or penetrate prior to their administration, e.g., but not limited to, nanoparticles.

### Bioreactors and Automation

One or more steps for the culture of stem cells and/or differentiation of NK cells from pluripotent cells may be automated. Automating a process using robotic or other automation can allow for more efficient and economical methods for the production, culture, and differentiation of cells. For example, robotic automation may be utilized in conjunction with one or more of the culture of human embryonic stem cells, passaging, addition of media, addition of differentiation media, culture in differentiation media, and separation of cell type, e.g., using magnetic separation or FACS.

A bioreactor may also be used in conjunction with the present embodiments to culture, maintain, and/or differentiate any of the various cells disclosed herein according to the present embodiments. Bioreactors provide the advantage of allowing for the scaling up of a process, wherein an increased amount of cells is produced. Non-limiting examples of bioreactors are batch bioreactors, fed batch bioreactors, continuous bioreactors, and/or a chemostat.

Robotic automation for use with the present disclosure may include liquid handling tools such as cap-piercing probes and disposable tips to minimize carry-over between samples. In some embodiments, robotics may be utilized in conjunction with one or more bioreactor for culturing cells disclosed herein. In certain embodiments, cells of the present disclosure may be cultured on a robot. The maintenance, seeding, feeding and/or harvesting of the EBs may be partially or completely automated.

In some embodiments, it may be useful to miniaturize or scale down methods of the present embodiments. These approaches may be particularly useful, e.g., where the methods comprise a high-throughput screen of compounds, e.g., which may promote de-differentiation or differentiation of cells towards a particular lineage. High-throughput screens may also be used to evaluate one or more property of a candidate substance (e.g., toxicity, ability to promote or reduce differentiation, etc.).

### EXAMPLES

The present disclosure is also described and demonstrated by way of the following examples. However, the use of this and other examples anywhere in the specification is illustrative only and in no way limits the scope and meaning of the disclosure or of any exemplified term. Likewise, the disclosure is not limited to any particular preferred embodiments described here. Indeed, many modifications and variations of the disclosure may be apparent to those skilled in the art upon reading this specification, and such variations can be made without departing from the disclosure in spirit or in scope.

### Example 1. Generation of Induced Pluripotent Stem Cells (iPSCs

Several cell types such as, but not limited to, peripheral blood mononuclear cells (PBMCs), peripheral blood natural killer cells (PBNKs), skin (epithelial) cells, fibroblasts, and fat cells (adipocytes), may be useful in the production of human induced pluripotent stem cells (iPSCs) by reprograming. Further, iPSCs may be produced by the induction of expression of a number of embryonic genes. As a non-limiting example, a mixture of vector(s) carrying genes Oct3/4 (Pou5fl), Sox2, Klf4, and c-Myc (Yamanaka factors), as well as a mixture of vector(s) with genes Oct3/4, NANOG, SOX2 and LIN28 (ONSL) (Thomson cocktail), may be used for transduction. Several other combinations of genes are also possible such as, but not limited to, Oct4, Sox2, Nanog, and cMyc [42]). In certain instances, iPSC lines may be derived from MRC-5 fibroblasts (ATCC CCL-171) and AG06872 fibroblasts (Coriell Institute) by over-expressing Oct4, Sox2, Nanog, and cMyc using retroviral vector plasmids [42].

A combination of chemical stimuli may also be used toward induction of embryonic genes. Non-limiting examples of such chemical stimuli may include inhibitors of TGF-β receptor and MEK, e.g., SB-431542 and PD0325901. In some embodiments, the chemical stimuli that may be used toward induction of embryonic genes may be capable of improving the efficiency of the reprogramming >200-fold [43].

In the present experiment, iPSC lines were derived from peripheral blood mononuclear cells (PBMCs) or peripheral blood natural killer cells (PBNKs) using a CytoTune-iPSC 2.0 Sendai Reprogramming Kit (Thermo Fisher Scientific, MA) according to manufacturer's instructions. Briefly, PBMCs were isolated using BD Vacutainer CPT tubes and cultured for 3 days. On day 4 after isolation, 5×10⁵ of cells were plated into one well of a 12-well plate, and subsequently transduced with recommended target multiplicity of infections (MOIs) of polycistronic h-Klf4-h-Oct3/4-Sox2 (KOS), hc-Myc, and h-Klf4 (KOS MOI=5, hc-Myc MOI=5, hKlf4 MOI=5). PBMCs and PBNKs were preliminarily isolated and expanded.

On day 8 after transduction, the cells were plated onto 6-well vitronectin-coated plates, and monitored for the appearance of pluripotent colonies. Pluripotent clones were mechanically selected and expanded. Cell pluripotency was confirmed using immunofluorescence assessment of various markers including Oct4, Nanog, SSEA-4, TRA-1-60 and TRA-1-81 [44]. Fluorescence-Activated Cell Sorting (FACS) was used for analysis of cell surface antigen markers, e.g., SSEA-4, TRA-1-60.

A teratoma assay was used in some instances to provide an additional index of pluripotency. For the teratoma assay, approximately 1×10⁶ pluripotent cells were subcutaneously (s.c.) injected into the flank of NOD/SCID mice. After 5-8 weeks resultant teratomas were removed, fixed in 4% PFA, embedded in paraffin, and sectioned and stained with hematoxylin and eosin. Additional pluripotency confirmation strategies included PluriTest Whole Genome Expression Assessment (WGE) program (PluriTest.org), and RNA sequencing (RNAseq) using conventional methodology by those skilled in the art (performed at Northwestern University sequencing core NUseq), and as described in further detail below (see Example 5).

Various cell culture systems using Mouse Embryonic Fibroblasts (MEFs) or feeder-free approaches were used for the maintenance of human pluripotent stem cells (hPSCs), including human embryonic stem cells (hESCs) and human induced pluripotent stem cells (iPSCs) disclosed herein. As a non-limiting example, the composition of media for hPSC co-cultured with mouse embryonic fibroblast (MEF) cells may comprise of K-DMEM medium supplemented with 20% fetal bovine serum (FBS), or its Knockout Serum Replacement (SR-1), and additionally supplemented with 2 mM β-mercaptoethanol and recombinant human fibroblast growth factor (e.g., basic fibroblast growth factor) bFGF at a concentration of 5 ng/ml.

### Example 2. Hematopoietic Differentiation of Human Pluripotent Stem Cells (hPSCs

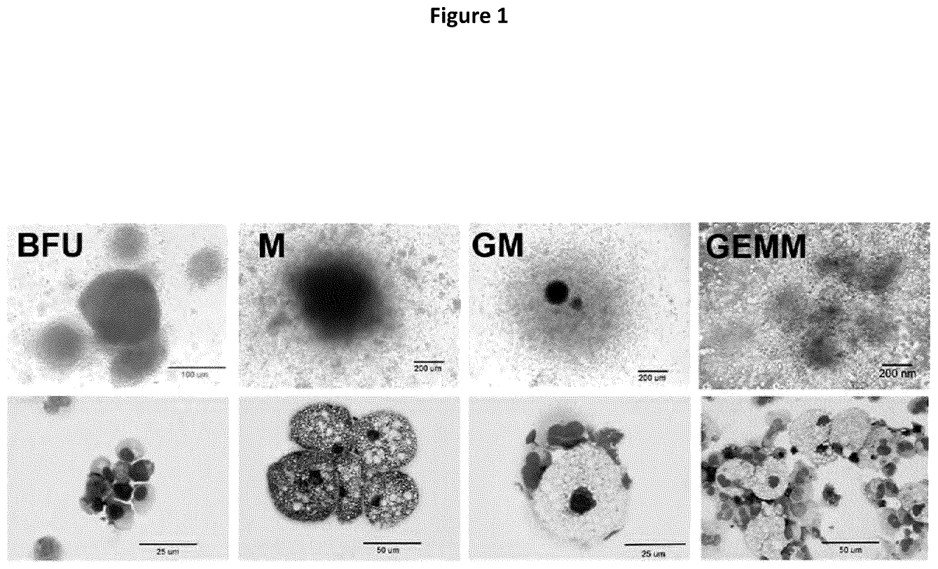

Hemogenic endothelium (HE) differentiation was established by a monolayer induction protocol. Single cells were plated onto 60 mm culture dishes coated with Matrigel (Corning, MA) and cultured overnight in iPS-Brew (Miltenyi Biotec, California), or mTeSR1 (StemCell Technologies, MA). Differentiation was induced with an induction media containing advanced DMEM/F-12 (Thermo Fisher Scientific, MA), glutamax (2.5 mM) (Thermo Fisher Scientific, MA), ascorbic acid (60 μg/mL) (MilliporeSigma, MO) and CHIR99021 (2-9 μM; Tocris Bioscience, MN) added on day 0. On day 2 of induction, CHIR99021 was removed from the media. Vascular endothelial growth factor (VEGF) (Peprotech, NJ) was then added at a concentration of 20-100 ng/mL. A series of photomicrographs demonstrating multipotent hematopoietic progenitors and respective blood cell types that may be generated using methods described in the present disclosure are shown in FIG. 1. Specifically, the images capture a hematopoietic colony forming unit (CFU) assay, and corresponding cell morphologies, e.g., erythroid (BFU), macrophage (M), granulocyte-macrophage (GM), and granulocyte, erythroid, monocyte and megakaryocyte (GEMM). Methods disclosed herein significantly reduce the complexity of current protocols known in the art for hematopoietic induction. Further, the disclosed methods offer a defined system to study the factors that affect the early stages of hematopoiesis, as well as provide an optimized route of lymphoid and myeloid cell derivation from human pluripotent stem cells, thereby enhancing their use in translational medicine. Unlike other protocols known in the art, methods of the present disclosure also does not require the addition of cytokines such as, but not limited to VEGF, which is optional.

### Example 3. Differentiation of Natural Killer (NK) Cell in a Three-Dimensional Vascular Organoid System

In the present example, supporting cells produced using a CHIR99021 induction monolayer system were utilized to create a three-dimensional (3-D) vascular organoid capable of producing highly pure and functional NK cells. Cell density and composition were critical for the establishment of 3-D vascular organoids.

Human pluripotent stem cells (e.g., H1 human embryonic stem cells, H1 ESC or iPSCs) were plated at a density 1×10⁶ cells onto a 60 mm cell culture dish. After 5 days of differentiation under CHIR99021 (3 μM-8 μM) plus VEGF (20 ng-100 ng/mL; e.g., see Example 2), 12×10⁶ CHIR99021-induced cells were used to form pre-vascular cell clusters containing vascular progenitors and stromal cells. The clusters were formed by detaching cell layers enzymatically with, e.g., Accutase or Trypsin (e.g., 0.05% Trypsin), or mechanically using StemPro EZPassage (Thermo Fisher Scientific, MA) and a cell lifter (MilliporeSigma, MO) in Ca²⁺/Mg²⁺-free PBS. Approximately 20-30% of cells comprising a cluster were CD31/CD34/CD144 triple-positive cells.

Pre-vascular clusters consisting of several thousand cells were then plated onto fibronectin coated-dishes into alpha-MEM media containing 10% Fetal bovine serum (FBS), monothioglycerol (MTG) (50-150 mM), stem cell factor (SCF; 10-60 ng/mL), retinoic acid (0.01-0.08 μM), ascorbic acid (20-100 μg/mL). Alternatively, pre-vascular clusters were plated onto a confluent monolayer of feeder cells such as, but not limited to, OP9-DLL4 feeder cells (obtained from Dr. Igor Slukvin) [47]. Twenty-four hours after adhesion of the clusters to the cell culture dish, the differentiation media was supplemented with IL-3 (R&D Systems, MN) and Fms-like tyrosine kinase 3 ligand (FLT3-L) (R&D Systems, MN).

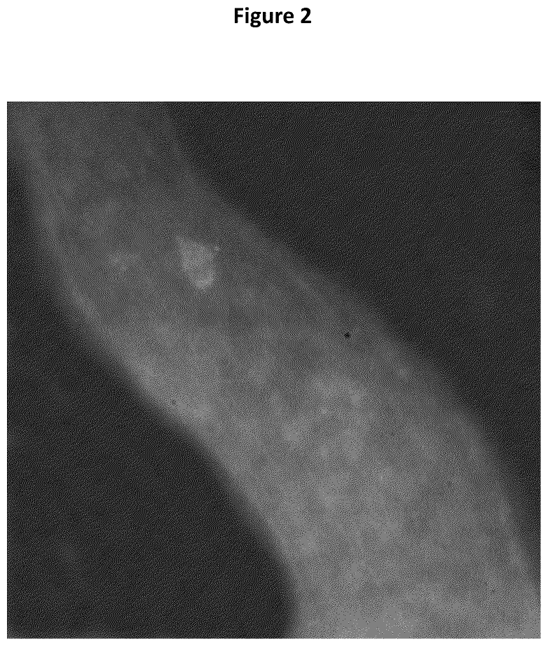

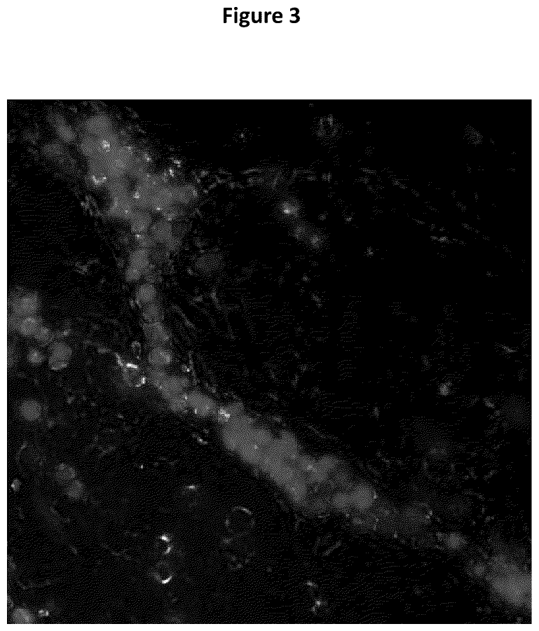

After the vascular organoids were formed, approximately 72 hrs after plating, the clusters, IL-7, and IL-15 were added to differentiation media. The hematopoietic progenitors that developed inside the vascular organoid became CD43⁺ on day 5-9 after plating. An exemplar image showing hematopoietic progenitor cells expressing CD43 that developed inside of a vascular organoid formed by a tube of endothelial cells is shown in FIG. 2. As represented in FIG. 3, hematopoietic progenitor cells expressing CD43 developed and flowed along the vascular organoid. The floating hematopoietic progenitors, including hematopoietic stem cells (HSC), emerged from the vascular organoid starting on day 5-7 of differentiation. On day 12 of differentiation, floating cells were collected and replated onto fresh fibronectin-coated dishes into media containing 10-50 ng/mL SCF, 10-50 ng/mL FLT3-L, 10-50 ng/mL IL-7, 10-50 ng/mL 10-50 ng/mL IL-15 (R&D Systems, MN) and 3 μM-8 μM CHIR99021, without IL-3, for 10-14 more days for NK cell maturation. Alternatively, on day 12 of differentiation, floating cells were collected and replated onto the semi-confluent layer of OP9-DLL4 cells into media containing cytokines and CHIR99021, without IL-3, for 10-14 more days for NK maturation.

The vascular organoids were optionally formed by plating 5×10⁶ of CD31/CD34/CD144 triple positive clusters on 12-well plates with undiluted Martrigel cushion into alpha-MEM media containing 10% FBS and 5-15 ng/mL IL-3, 10-50 ng/mL SCF, 10-50 ng/mL FLT3-L, 10-50 ng/mL IL-7, 10-50 ng/mL IL-15.

The vascular organoids disclosed herein allowed for proper development and functional maturation of NK cells, similar to that which occurs in vivo. Outstanding results were observed when using protocols of the present disclosure to differentiate 1×10⁶ H1 ESC- or 1×10⁶ iPSC-derived NK cells. 47×10⁶ CD56+ cells with 90% purity were produced in 24 days, 16% of which expressed CD16. These cells were negative for CD3 expression, and were further expanded by 10³-10⁴ times depending on stimulation method.

The exclusion of a cell selection step from the above-described methods step was advantageous as it abolished the risk for reduced efficiency via diminishing the progenitor cell population, and avoided deprivation of the developing cells from supportive concomitant stromal cells. The vessel organoid provided an optimal environment for the generation of progenitor cells, precisely recapitulating in vivo cellular organization, including signaling gradients and shear stress conditions, which have been shown to trigger a cascade of molecular events leading to the emergence of adult hematopoiesis [51].

### Example 4. Culture and Expansion of Natural Killer (NK) Cells

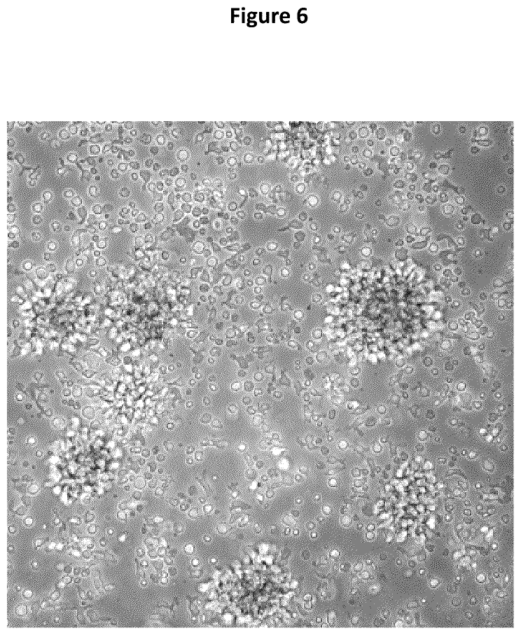

NK cells were cultured in RPMI-1640 supplemented with 10% FBS and 50 U IL-2. NK cells were then expanded up to 3×10⁴ times by weekly stimulation with IL-2 or by allogeneic feeder cells or their plasma membrane particles e.g., irradiated K562 (ATCC accession number CCL-243), IL-15- and/or 41BBL-expressing K562-mb15-41BBL [50], or IL-21 expressing K562 such as K562mbIl21 (obtained from Dr. Dean Lee) (1:2 ratio) [48]. Other suitable methods for NK stimulation, culture and expansion are discussed in [49]. A representative image of the morphologic appearance of iPSC-derived NK cells expanding in vitro as cell clusters is shown in FIG. 6.

### Example 5. Characterization of Natural Killer (NK) Cells

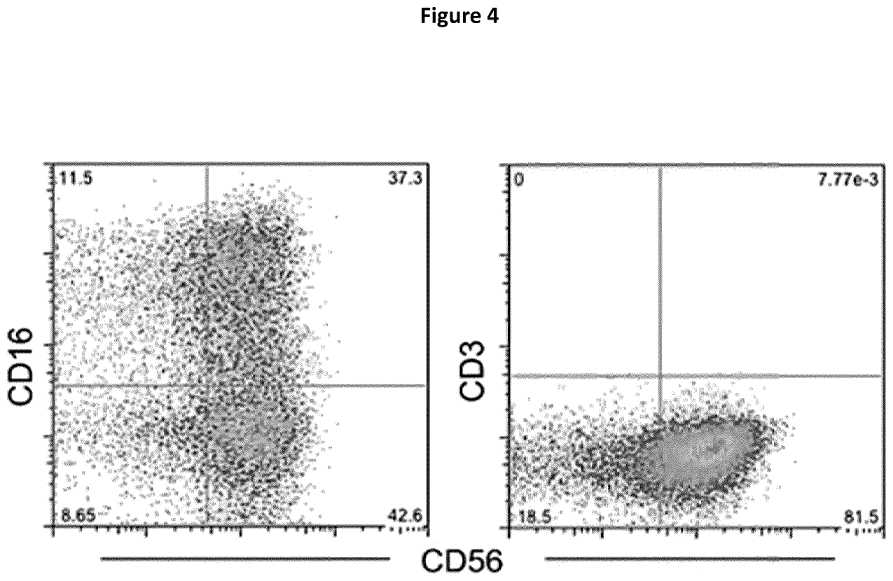

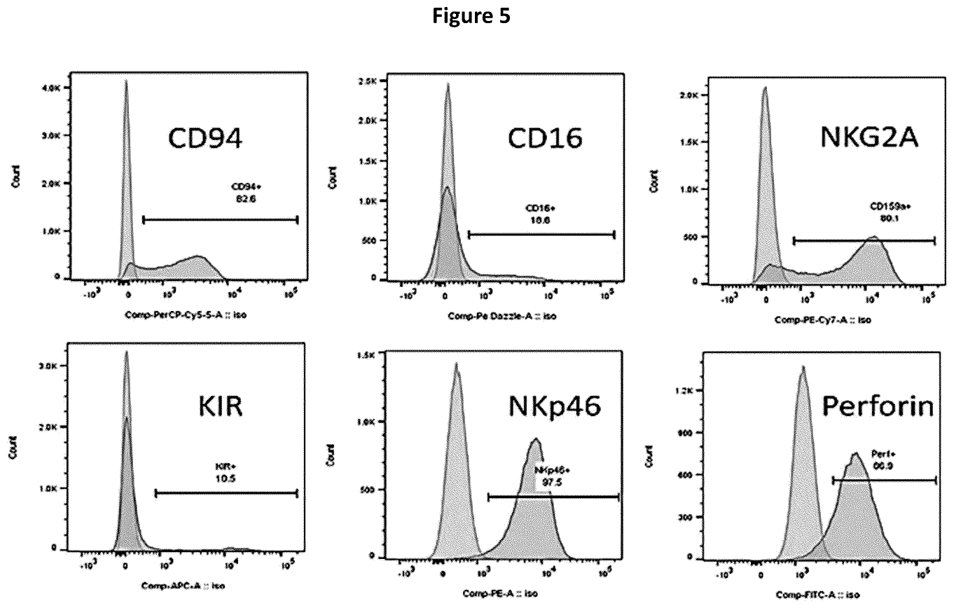

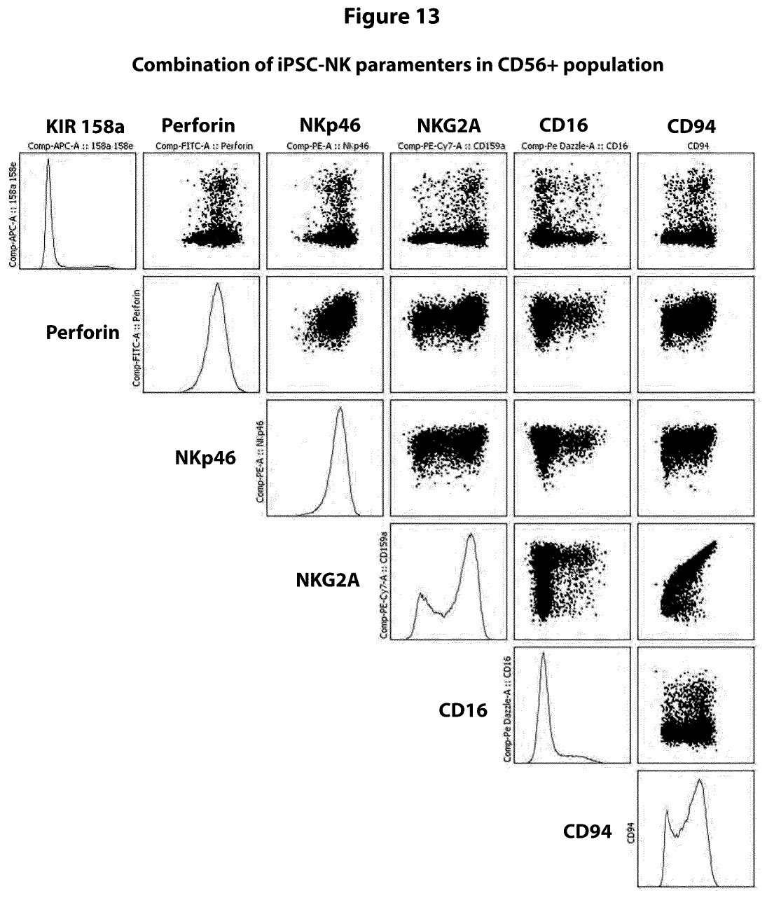

Several methodological approaches were used for the phenotypic characterization of the induced pluripotent stem cell (iPSC)-derived natural killer (NK) cells of the present disclosure. Specifically, flow cytometry analysis was used to characterize expression of surface markers such as, but not limited to, CD34, CD3, CD45, CD56, CD16, activating cytotoxicity receptors NKG2D, NKp46, FAS ligand, and inhibitory killer immunoglobulin-like receptors (KIRs). Exemplary flow cytometry assay data showing efficient production of NK cells (CD56⁺/CD16⁺/CD3⁻) from iPSCs using methods disclosed herein are shown in FIG. 4. Exemplary flow cytometry data showing functional markers CD94, CD16, NKG2A, KIR, NKp46, and perforin of iPSC-derived NKs are displayed in FIG. 5. A functional marker interface diagram showing iPSC-NK expression of functional markers activating cytotoxicity receptor NKp46, ADCC receptor CD16, inhibitory killer immunoglobulin-like receptors (KIRs), perforin, inhibitory receptors CD94, and NKG2A is displayed in FIG. 13. Of note, the expression of inhibitory killer immunoglobulin-like receptors (KIRs) in NK cells disclosed here is minor, in support of superior functionality of these cells upon transplantation to a wide range of genetically diverse recipient patients.

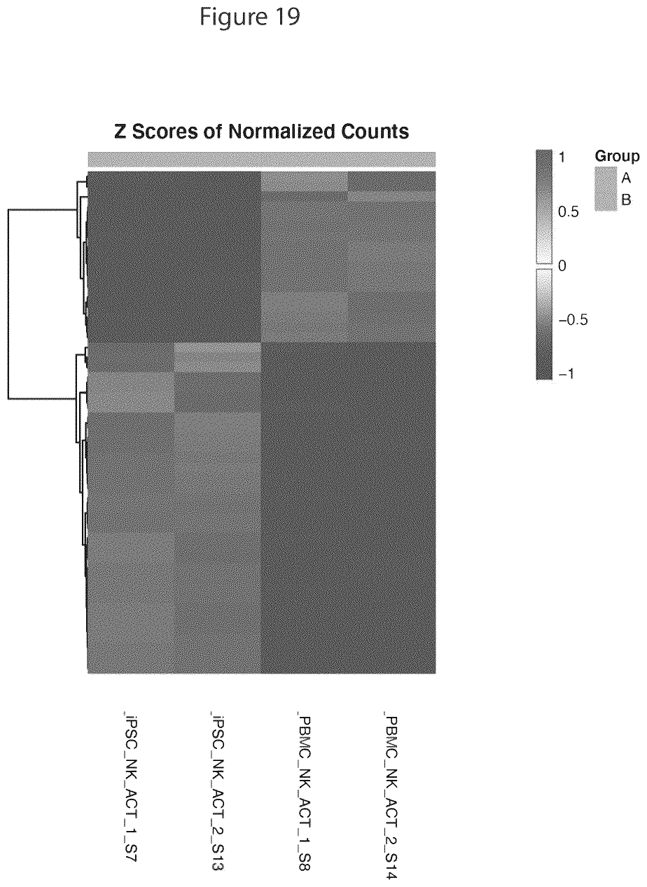

Produced NK cells were additionally assayed by RNAseq for mRNA and non-coding RNAs. Exemplary RNAseq results are displayed in FIG. 19. Total RNA was extracted with the RNeasy Mini Kit (Qiagen) according to the manufacturer's protocol RNA quality and concentration were assessed with a Nanodrop instrument. Aliquots of RNA were submitted to Northwestern University's NUSeq Core. The total RNA library was prepared, and the samples were analyzed using HiSeq 4000 Sequencing (50 bp, Single Reads). The expression signature was compared between activated and non-activated NKs, as well as between hPSC Natural Killer cells and peripheral blood (PB) NKs, and confirmed the genuine phenotype of the hPSC-NKs. Furthermore, a STRING (Search Tool for the Retrieval of Interacting Genes/Proteins) biological database analysis revealed a number of functional processes that were up-regulated in hPSC-NK cells as compared to PB-NK cells, which were indicative of the superior regulatory function of hPSC-derived NK cells. As a non-limiting example, compared to PB-NK cells, hPSC-NK cells showed greater involvement in apoptotic cell clearance, negative regulation of vasculogenesis, cytokine secretion, regulation of immune response(s), as well as in immune cell proliferation, differentiation and cytotoxicity. Thus, these data show that iPSC-NKs of the present disclosure presents authentic markers and are functional.

Degranulation and cytokine secretion assays (see below for additional details) were used for the functional assessment of produced NK cells. For analysis of intracellular cytokines, samples were stimulated with Phorbol 12-myristate 13-acetate (PMA; 25 ng/ml) (MilliporeSigma, MO) and ionomycin (1 μM) (Fisher Scientific, MA) in presence of Brefeldin A (10 μg/ml) (StemCell Technologies, Ca) for 4 hrs at 37° C. with 5% C02. Following stimulation, cells were fixed and permeabilized using Cytofix/Cytoperm solution (BD Biosciences) for 20 min at 4° C. and stained with antibodies against, e.g., CD107a/perforin, TNF, and IFN-7 for 30 min at room temperature. The cells were then analyzed by flow cytometry (see, e.g., FIG. 5).

### NK Cell Degranulation Assay

Perforin and cytokines staining required intracellular staining. After staining for surface markers, cells were fixed and permeabilized using BD Cytofix/Cytoperm fixation permeabilization solution (Fixation/Permeabilization Solution Kit; BD Biosciences) according to the manufacturer's protocol for 20 min at 4° C. Then the cells then washed twice with the Perm/Wash solution (Fixation/Permeabilization Solution Kit; BD Biosciences) and stained with antibodies against perforin or cytokines for 30 min at room temperature (RT), washed with Perm/Wash solution, and analyzed on a flow cytometer.

For assessment of intracellular cytokines, the samples were stimulated for 4 h at 37° C. 5% CO₂ (in a CO₂ incubator) with PMA (25 ng/ml), ionomycin (1 μM), and in presence of Brefeldin A (10 ug/ml) (all from Sigma).

The procedure for cytokine (e.g., TNF and IFN-7) staining was as follows:
1. Stain the cells for surface antibodies.
2. Wash the cells with PBS (1×) two times.
3. Pellet the cells and add 250 μl of BD Cytofix/Cytoperm solution (BD Bioscience, catalog number 554722). Vortex the cells at 400 rpm while adding BD Cytofix/Cytoperm the solution.
4. Incubate the sample in the dark for 30 minutes at 4 C.
5. Add 2 mL of BD Perm/Wash (1×) (BD Biosciences, catalog number 554723). Centrifuge the sample at 1500 rpm for 5 min two times, consecutively.
6. Add 5 μl of each of TNF and IFN-7 antibodies (10 ng/μl) (BD Biosciences, CA). Incubate in the dark for 30-40 mins.
7. Wash two times with BD1 Perm/Wash (1×).
8. Add 500 μl of BD Perm/Wash (1×) and analyze using BD LSR II Flow cytometer.

### Cytotoxicity Assays

In order to evaluate the killing efficiency of NK cells against the K562 cell line, target cells were incubated with iPSC-NK at ratios of 1:25, 1:50 for 2 hours. Killing activity was assessed using Fluorescence-Activated Cell Sorting (FACS).

In order to evaluate the killing efficiency of NK cells against the DIPG SF8628 cell line in vitro, target cells were incubated with either PBMC-NK or iPSC-NK at ratios of 1:1, 1:5, and 1:10 for 4 hours. Killing activity was assessed using FACS analysis, as well as an automated cell counting method.

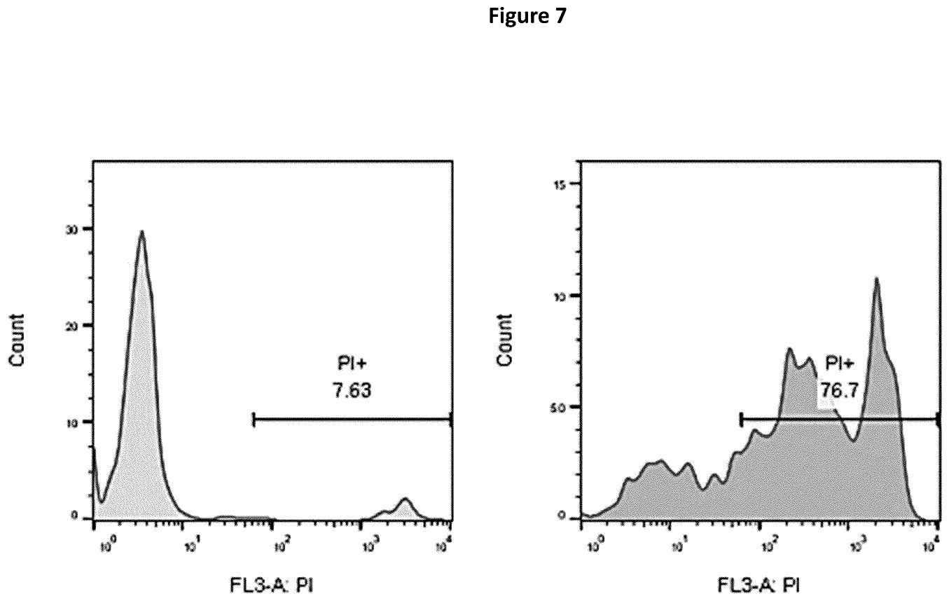

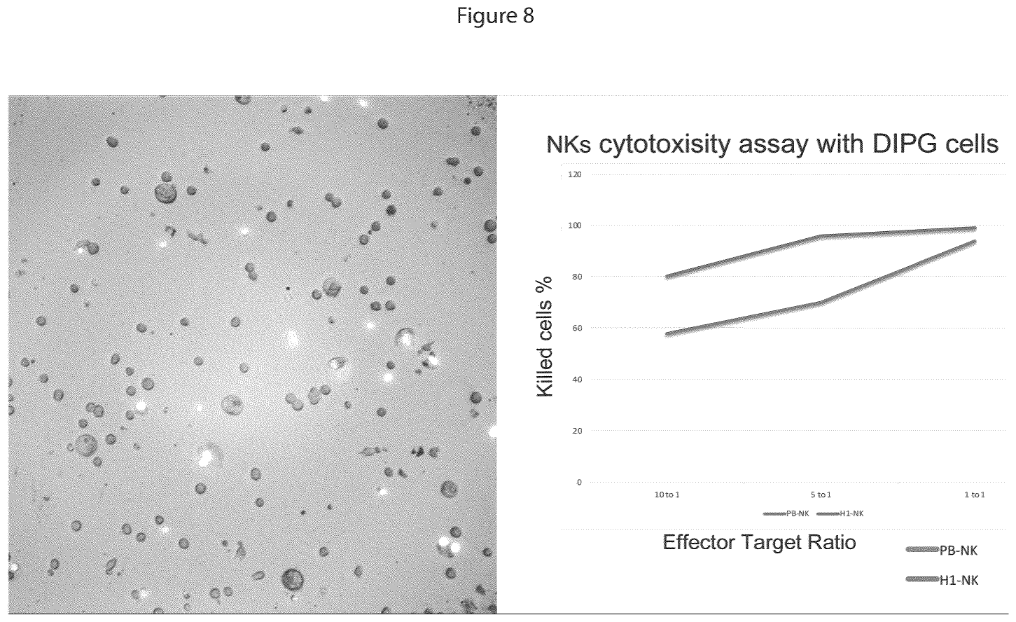

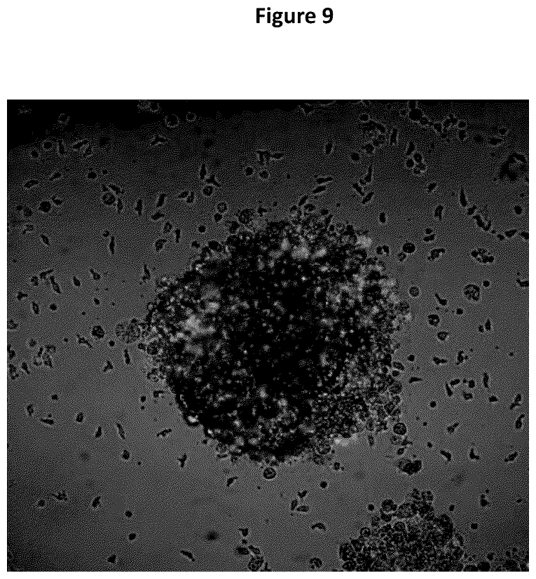

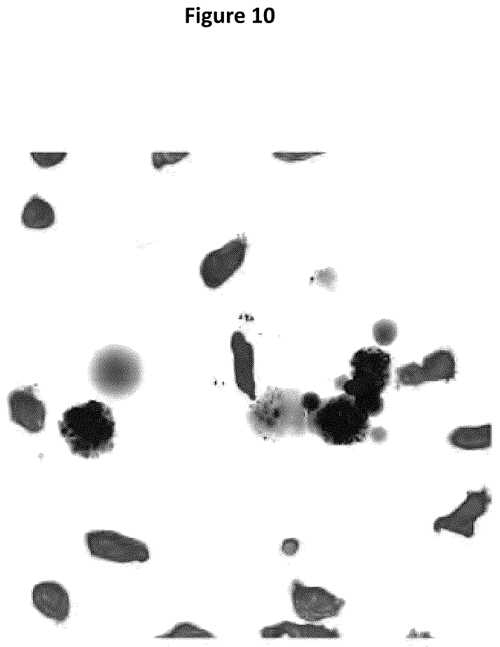

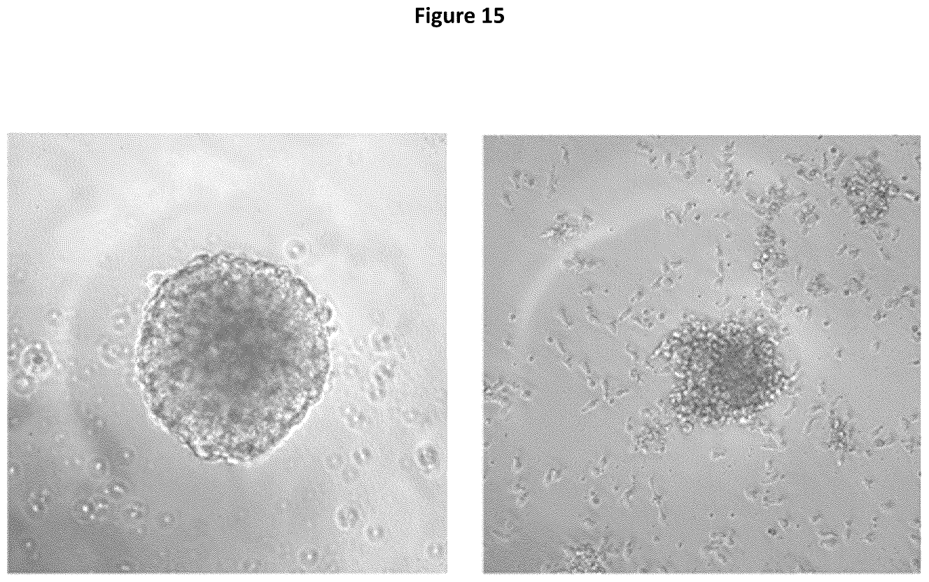

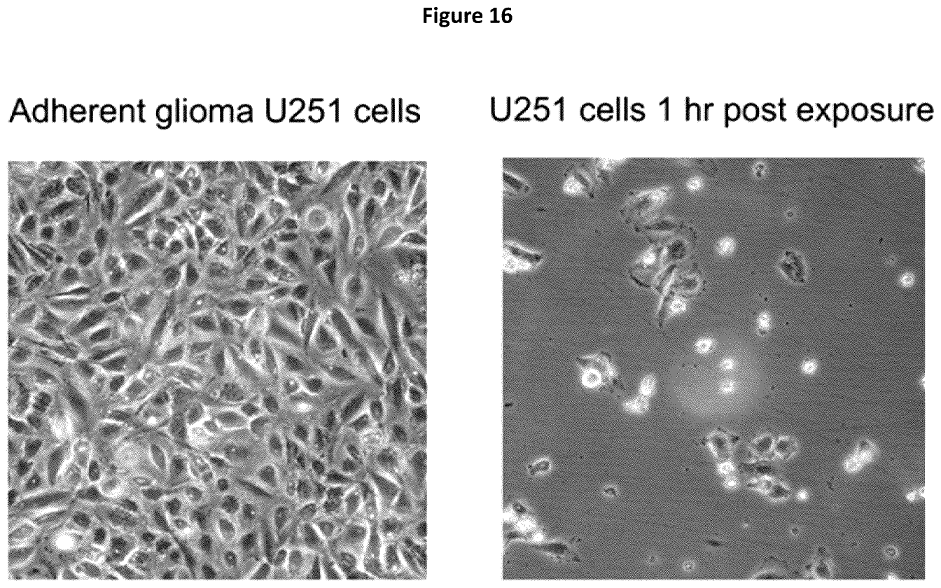

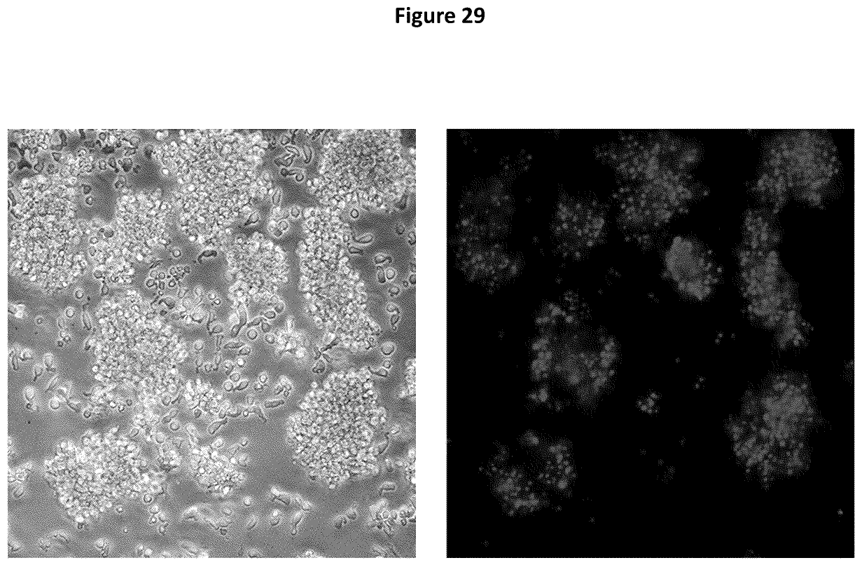

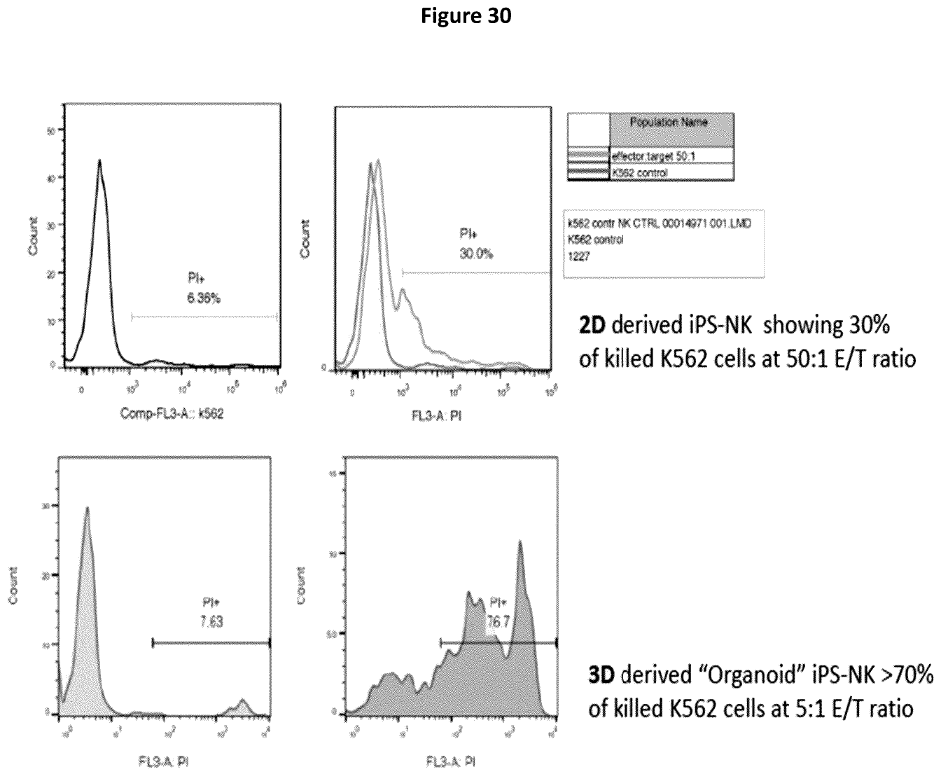

In vitro cytotoxicity assays against various cancer cells were used to interrogate cytotoxicity levels of NK cells. Specifically, in vitro cytotoxicity of NK cells was shown in adherent cultures, suspension cultures and 3-D tumor spheres against, e.g., AML K562 (FIG. 7 and FIG. 30), Jurkat (FIG. 29), adenocarcinoma, HeLa, glioblastoma (FIG. 9 and FIG. 15), glioma (FIG. 16), and SF8628 diffuse intrinsic pontine glioma (DIPG) (FIG. 8) cells. In vitro cytotoxicity of NK cells is further exemplified by the image displayed in FIG. 10, showing lysis of cancer cells upon 15 min interaction with iPSC-NKs.

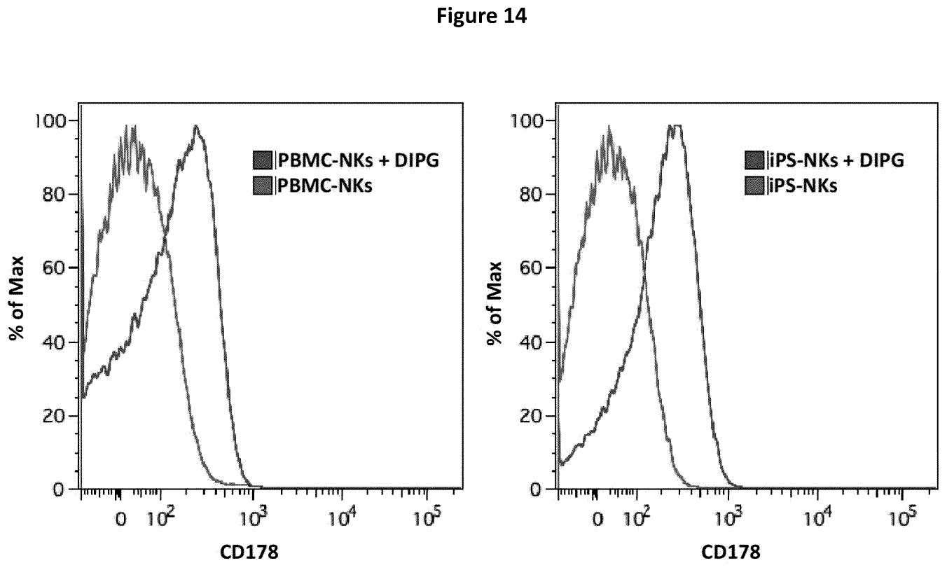

In order to evaluate the killing efficiency of NK cells against the DIPG SF8628 cell line (MilliporeSigma SCC127), in particular, target cells were incubated with either PBMC-derived NK (PBMC-NK) or iPSC-derived NK (iPSC-NK) cells at ratios of 1:1, 1:5, and 1:10 for 4 hours. Killing activity was assessed using FACS analysis, as well as an automated cell counting methods (FIG. 8). Both PBMC-NK and iPSC-NK were successful at killing the DIPG cells, with iPSC-NK performing at a higher efficiency of up to 80% at a 1:5 ratio. In addition, overnight incubation of NKs and DIPGs (SF8628) was shown to induce FasL expression (CD178), and both PBMC- and hPSC-derived NK cells responded to the DIPG stimulus, as indicated by the elevated cell numbers expressing FasL in comparison to untreated cells (FIG. 14). These data suggested that the iPSC-derived NK cells may provide a potential cellular therapeutic tool to combat DIPG and other brain cancers.

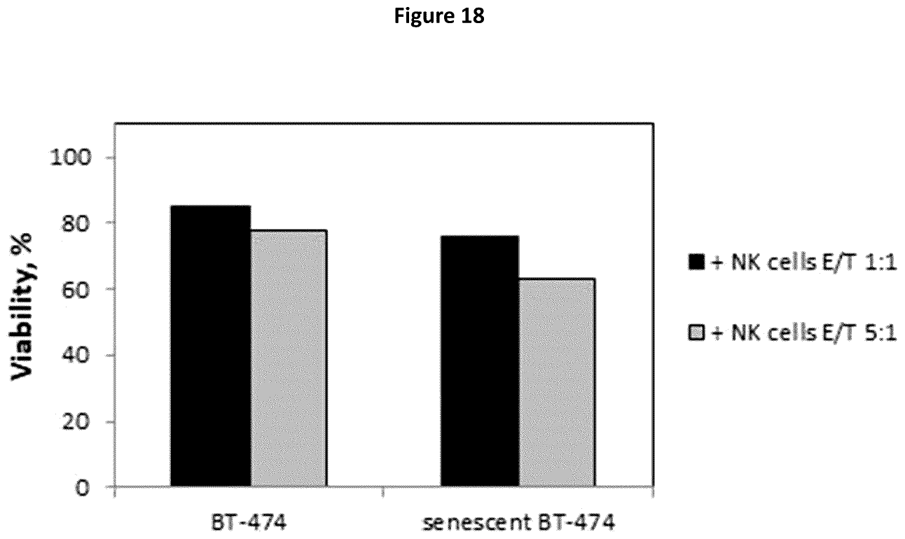

Senescent cells become immunogenic by expressing stimulatory ligands such as, but not limited to, MHC class I chain-related protein A and B (MICA/B) that bind to NKG2D receptor of NK cells and activate their killing activity. The enhanced lysis of BT-474 senescent cells by NKs as compared to control cells is shown FIG. 18.

The above-described results demonstrate a method of NK differentiation that allows for derivation of highly-functional NKs possessing both cytotoxic and regulatory capacities.

### Example 6. KLRC1 Gene (NKG2; NKG2A; CD159A) Knockout in hPSC Using the CRISPR-Cas9 Editing System

Methods for the present example included the following steps: 1. Selection of guide RNA (gRNA) sequences specific to different sites of the KLRC1 gene; 2. Construction of expression plasmid vectors encoding the selected gRNA and Cas9 endonuclease; 3. hPSC transfection; 4. Selection of knockout clones with PCR and fluorescence in situ hybridization (FISH); 5. Knock out confirmation by sequencing; 6. Differentiation of pluripotent clones into NK cells; 7. NKG2A expression verification by immunoblotting with anti-NKG2A antibodies.

To select the optimal gRNA sequences, the analysis of nucleotide sequences was carried out using web resources: CRISPOR (crispor.tefor.net), CRISPR Design (crispr.mit.edu), CHOPCHOP (chopchop.cbu.uib.no). Sequences of gRNAs (SEQ ID NO: 1, SEQ ID NO: 2) specific to various sites of the KLRC1 gene (SEQ ID NO: 3, SEQ ID NO: 4) were selected.

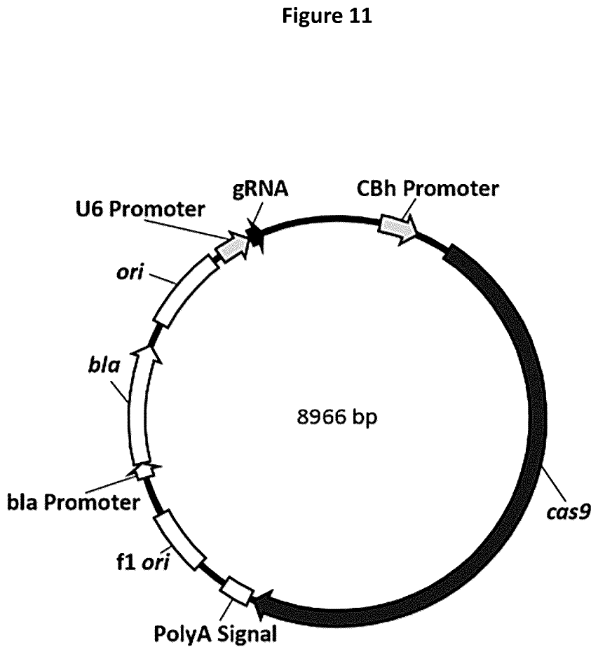

To obtain plasmid vectors encoding complexes of Cas9 endonuclease and gRNAs, DNA fragments encoding gRNAs were synthesized using PCR with overlapping oligonucleotides. The resulting fragments were cloned into a plasmid vector intended for the expression of the components of the CRISPR-Cas9 system in mammalian cells. The vector was digested at the BbsI restriction endonuclease site. Selected plasmid vectors pGR-NKG2A-1 and pGR-NKG2A-2 were sequenced to confirm the receipt of the planned genetic constructs. A general map of vectors pGR-NKG2A-1 and pGR-NKG2A-2 is shown in FIG. 11.

Human pluripotent stem cells were transfected with vectors pGR-NKG2A-lor pGR-NKG2A-2 using Lipofectamine 3000 (Thermo Fisher Scientific, MA). A mixture of Lipofectamine 3000 and DNA was prepared (at concentrations of 3 μl lipofectamine, 4 μg pGR-NKG2A-1 or pGR-NKG2A-2, 8 μl P3000™ reagent, in 100 μl Opti-MEM® Medium per 1 well of a 12-well plate). The mixture was incubated for 10 min and added to the well. The cells were incubated for 48 h at 37° C. and 5% C02. Alternatively, cells were transfected with vectors pGR-NKG2A-1 or pGR-NKG2A-2 using electroporation system Amaxa (Lonza, NJ), program 1 for stem cells, according to the manufacturer's instructions.

The transfection was repeated 4 times after intermediate passaging of cells 5-7 times in 6-well plates.

Twenty-four hours after the last transfection, the colonies were detached and separated into single cells using Accutase. Single cells were transferred into the wells of a 96-well plate and incubated for 7 days at 37° C. and 5% CO₂ before splitting into two wells. Clones were analyzed by real-time PCR with fluorescent probes specific to target sites of guide RNAs in the KLRC1 gene. KLRC1 knockout hPSC cells were selected for the absence of a fluorescent signal. Alternatively, clones were be selected by fluorescence in situ hybridization (FISH).

Selected clones were confirmed for KLRC1 knockout by RNA-sequencing.

hPSC were then differentiated to NK cells using methods similar to or the same as those described in the above Examples 1-5.

The lack of NKG2A expression in differentiated hPSC-NK clones was further confirmed by Flow cytometry or immunoblotting with anti-NKG2A antibodies. Non-transfected cells were used as a positive control.

To evaluate NKG2A expression by flow cytometry, cells were incubated with human NKG2A/CD159a APC-conjugated antibody for 1 h. The cells were washed with 0.5% BSA in PBS and analyzed by Flow cytometry.

To evaluate NKG2A expression by immunoblotting, cells were incubated with human NKG2A. The cells were lysed using a buffer containing 25 mM Tris-HCl, 150 mM NaCl, 1 mM EDTA, 1% Triton-X100. Cell lysates were centrifuged at 10,000 g for 10 min. The concentration of total protein in the supernatant was determined, and aliquots corresponding to 100 μg of protein were separated by electrophoresis under denaturing conditions. Proteins were electrically transferred from the gel to a nitrocellulose membrane. The nitrocellulose membrane with immobilized proteins was then washed with buffer I (20 mM Tris-HCl, pH 7.5; 150 mM NaCl; 0.05% Tween-20) and incubated for 1 h in 2% bovine serum albumin (BSA) in buffer I. Nitrocellulose membranes were then incubated for 16 hrs with primary antibodies to NKG2A in 2% BSA in buffer I. After that, the membrane was washed with buffer I and incubated for 1 h with secondary antibodies conjugated with horseradish peroxidase in 1% milk solution in buffer I. The membrane was washed with buffer I and stained using a commercial ECL™ chemiluminescent detection kit. Clones were selected that did not show specific bands corresponding to NKG2A.

The scheme of vectors pGR-NKG2A-1, pGR-NKG2A-2, was as follows: 295-572 bp—CBh promoter; 819-5090 bp—endonuclease SpCas9; 5121-5328 bp—PolyA Signal; 5552-6007 bp—f1 ori; 6289-6393 bp—bla promoter; 6394-7254 bp—bla; 7425-8013 bp—ColE1 ori; 8075-8315 bp—U6 promoter; 8322-8419 bp—a guide RNA coding sequence. Alternatively, vectors may include sequences for antibiotic resistance, fluorescent proteins e.g. GFP or other selection markers (FIG. 11).

Sequences encoding guide RNAs:

In pGR-NKG2A-1 vector
SEQ ID NO: 1
TGAAGGTTTAATTCCGCATGTTTTAGAGCTAGAAATA
GCAAGTTAAAATAAGGCTAGTCCGTTATCAACTTGAA
AAAGTGGCACCGAGTCGGTGC
In pGR-NKG2A-2 vector
SEQ ID NO: 2
CGTTGCTGCCTCTTTGGGTTGTTTTAGAGCTAGAAAT
AGCAAGTTAAAATAAGGCTAGTCCGTTATCAACTTGA
AAAAGTGGCACCGAGTCGGTGC
Target DNA sequences (PAM sequences highlighted)
For vector pGR-NKG2A-1
SEQ ID NO: 3
TGAAGGTTTAATTCCGCATAGG
For vector pGR-NKG2A-1
SEQ ID NO: 4
CGTTGCTGCCTCTTTGGGTTTGG

### Example 7. Suppression of NKG2A Expression in iPSC-NK Cells and PB-NK Cells Using siRNA

Methods for the present example included the following steps: 1. Selection of siRNA sequences specific to different sites of NKG2A mRNA; 2. Annealing pairs of chemically synthesized oligonucleotides (oligos) to obtain siRNA duplexes; 3. Transfection of NK cells with siRNA duplexes; 4. NKG2A expression verification by Flow cytometry or immunoblotting with anti-NKG2A antibodies.

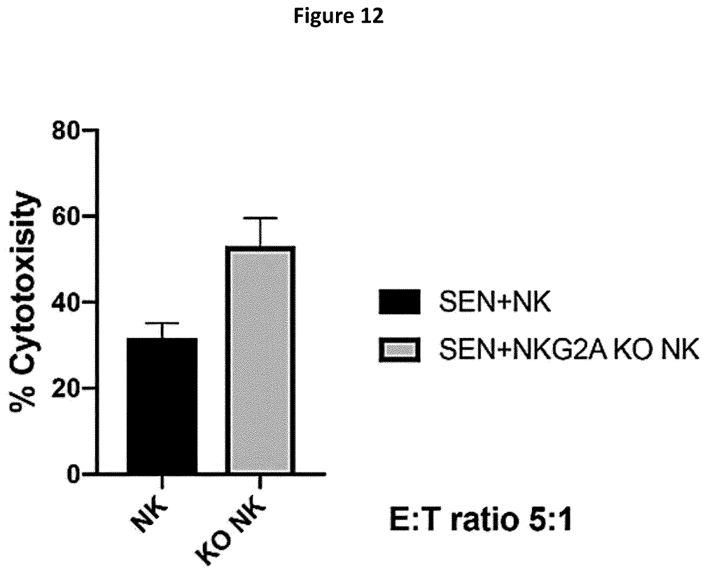

Transient suppression of NKG2A expression using siRNA was used on differentiated NK cells or peripheral blood derived NK cells to prepare cell batch for cell therapy or in vitro experiments. Results of an in vivo cytotoxicity experiment showing increased cytotoxicity of modified NKG2A KO NKs over wild type (NKG2A intact) iPSC-NK cells after 4 hr incubation with senescent IRM-90 fibroblasts (E:T ratio 5:1) at day 7 after 10 Gy irradiation are shown in FIG. 12.

To select the optimal siRNA, the analysis of the nucleotide sequences of KLRC1 mRNA was performed using web resources: siRNA Wizard (invivogen.com/sirnawizard/), siDirect (sidirect2.rnai.jp/). RNA oligo sequences (SEQ ID NO: 5-8) were selected specific for different target sequences (SEQ ID NO: 9, SEQ ID NO: 10).

To obtain siRNA duplexes, the corresponding pairs of oligonucleotides (final concentration 20 μM) were mixed in a buffer containing 20 mM Potassium Acetate, 6 mM HEPES at pH 7.4, 0.4 mM Magnesium Acetate. The mixture was incubated for 3 min at 90° C., then slowly cooled within 60 min to room temperature.

The siRNA transfection was performed using Lipofectamine 3000 (Thermo Fisher Scientific). A mixture of Lipofectamine 3000 and DNA was prepared (at concentrations of 3 μl of Lipofectamine 3000, 4 μg of siRNA, 100 μl of Opti-MEM® Medium per 1 ml of NK cell suspension). The mixture was incubated for 10 min and added to NK cell suspension. The cells were incubated for 48 h at 37° C. and 5% C02.

To evaluate NKG2A expression 48 hrs after transfection, NK cells were incubated with human NKG2A/CD159a APC-conjugated antibody for 1 hr. The cells were washed with culture medium and analyzed by Flow cytometry. Non-transfected cells were used as a positive control. Table 1 displays a description of RNA oligonucleotide sequences and target sequences useful for the methods described in the present example.

TABLE 1
RNA oligonucleotide sequences
and target sequences

| SEQ ID NO | Name | Sequence 5′→3′ |
|---|---|---|
| SEQ ID NO: 5 | guide | UUAAUUCCGC |
| | siRNA#1 | AUAGGUUAUU |
| | | UCdTdT |
| SEQ ID NO: 6 | passenger | GAAAUAACCU |
| | siRNA#1 | AUGCGGAAUU |
| | | AAdTdT |
| SEQ ID NO: 7 | guide | AAGAUAAGAC |
| | siRNA#2 | AGAUAAUUCC |
| | | CdTdT |
| SEQ ID NO: 8 | passenger | GGGAAUUAUC |
| | siRNA#2 | UGUCUUAUCU |
| | | UdTdT |
| SEQ ID NO: 9 | target | GAAATAACCT |
| | #1 | ATGCGGAATT |
| | | AA |
| SEQ ID NO: 10 | target | GGGAATTATC |
| | #2 | TGTCTTATCT |
| | | T |

### Example 8. In Vivo Cytotoxicity of NK Cells Against Acute Myeloid Leukemia

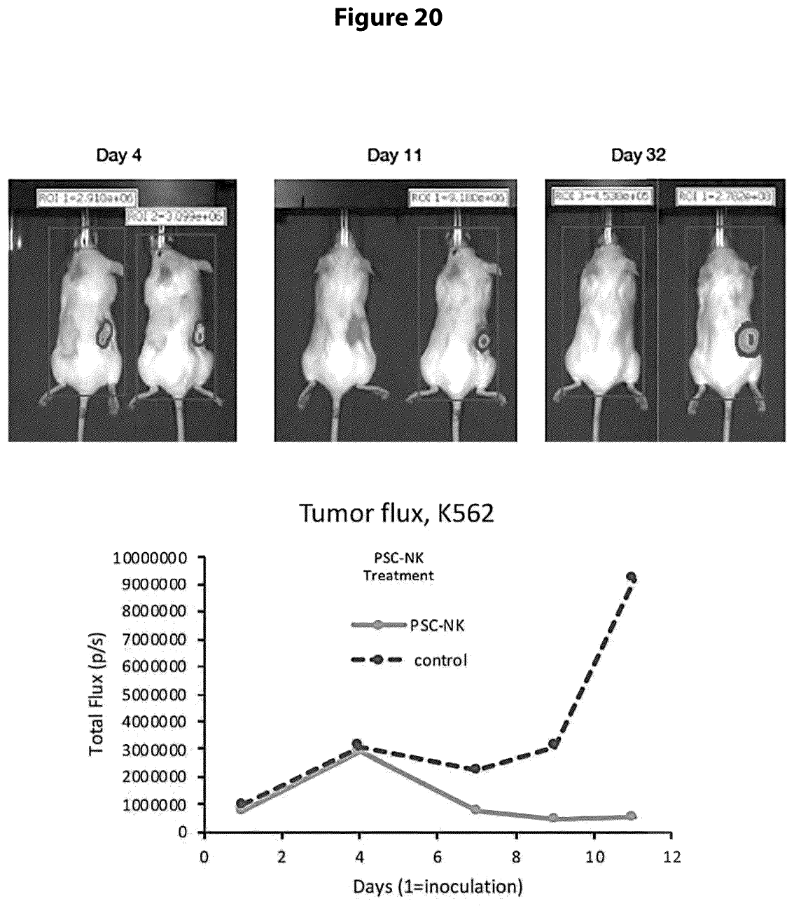

In vivo studies to investigate cytotoxicity of NK cells against Acute Myeloid Leukemia (AML) were performed using immunodeficient NOD/SCID mice injected with luciferase reporter cell line-K562 (CML, chronic myeloid leukemia; ATCC accession number CCL-243). In this model, mice received subcutaneous (s.c.) injection of tumor cells (5×10⁶ cells) into the right dorsal flank. Four days after injection of the tumor cells, mice in the NK cell treatment group were additionally injected (s.c., around the tumor site) with iPSC-derived NK (iPSC-NK) cells (1×10⁷ cells), whereas control mice received vehicle (phosphate buffered saline, PBS) injection. Tumor growth and metastases were monitored via bioluminescence imaging using a Perkin Elmer IVIS Spectrum imaging system. Visualization of bioluminescence revealed tumor killing efficacy of NK cells to at least one month post-inoculation (FIG. 20, top panel). Tumor flux data collected from day 1 (inoculation) through day 11 post-inoculation demonstrated reduced tumor progression in mice that received NK cell treatment as compared to control mice (FIG. 20, bottom panel). The above-described in vivo studies were performed at the Developmental Therapeutics Core (DTC) of Ann & Robert Lurie Comprehensive Cancer Center of Northwestern University.

### Example 9. Hematopoietic Progenitor Cell (HPC) Engraftment

While the differentiation of various types of hematopoietic stem cells (HSCs) from human pluripotent stem cells (hPSCs) has been carried out with some success, the generation of engraftable hematopoietic progenitor cell (HPCs) remains an ongoing pursuit [52, 53]. The molecular signature of engraftable cells were investigated in several studies [54, 55]. Overexpression of seven transcription factors (ERG, HOXA5, HOXA9, HOXA10, LCOR, RUNX1 and SPI1) was sufficient for conversion of hemogenic endothelium (HE) into hematopoietic stem cells (HSCs) that engraft in primary and secondary mouse recipients [55].

A long-term engraftment at 4 months of human induced pluripotent stem cell (iPSC)-derived CD45-negative hematopoietic progenitors was also detected after transplantation of human iPSC-hemogenic endothelial cells into the livers of fetal sheep [56]. Though human CD45⁺ cells were not detected in either peripheral blood or bone marrow with cytometric analyses, engraftment was detected by performing a colony-forming unit (CFU) assay with bone marrow cells, and nested polymerase chain reaction (PCR) analysis for the human mitochondrially encoded NADH:ubiquinone oxidoreductase core subunit 5 (ND5)-specific gene.

Derivation of engraftable HPCs may be especially crucial for therapies involving HPC transplantation, such as cancer therapy, gene therapy, and therapies of autoimmune diseases. A focal point of stem cell transplant is reducing the toxicity of this highly sophisticated treatment by minimizing graft-versus-host disease (GVHD), which may be achieved via derivation of patient-specific HPCs from PSCs, e.g., iPSCs and hPSCs, including at the hemogenic endothelium (HE) stage, as described in the present example. Specifically, the below experiments describe a procedure for the production of engraftable hematopoietic progenitor cells (HPCs; Hematopoietic stem cells, HSCs), and demonstrate the superior and unexpected finding of engraftment of such cells at several stages of differentiation, including cells conditioned on OP9 cells and within vascular organoids of the present disclosure, as well as CD34⁺ progenitor cells, which do not yet express hematopoietic markers, e.g., CD43 or CD45.

This work evaluated the capability of an exemplary differentiation system to produce engraftable cells. To determine which differentiation point(s) resulted in engraftment, differentiation conditions were analyzed at any of various time points, e.g., at approximately 4-7 days, 7-10 days, or 14-18 days following induction of differentiation. For each experiment, human cord blood isolated CD34⁺ cells served as control.

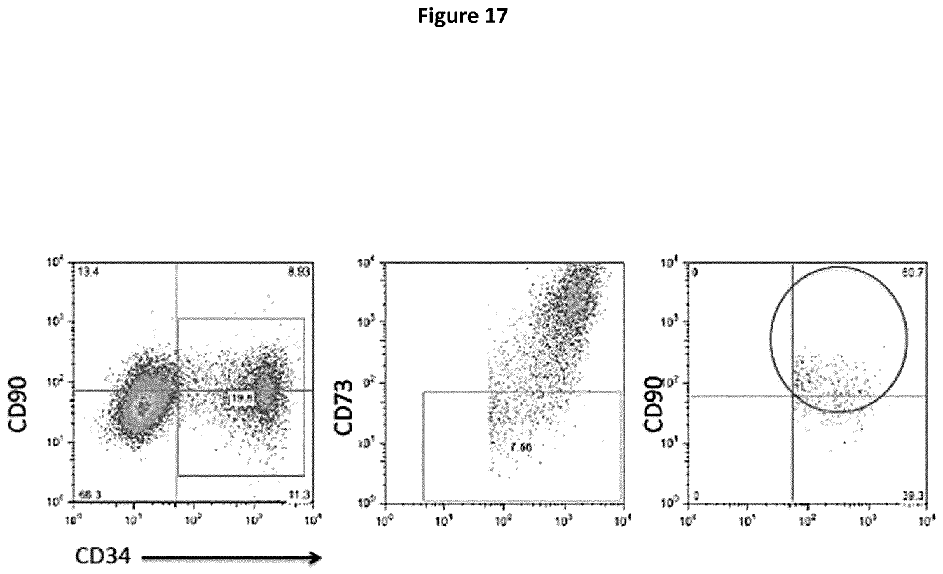

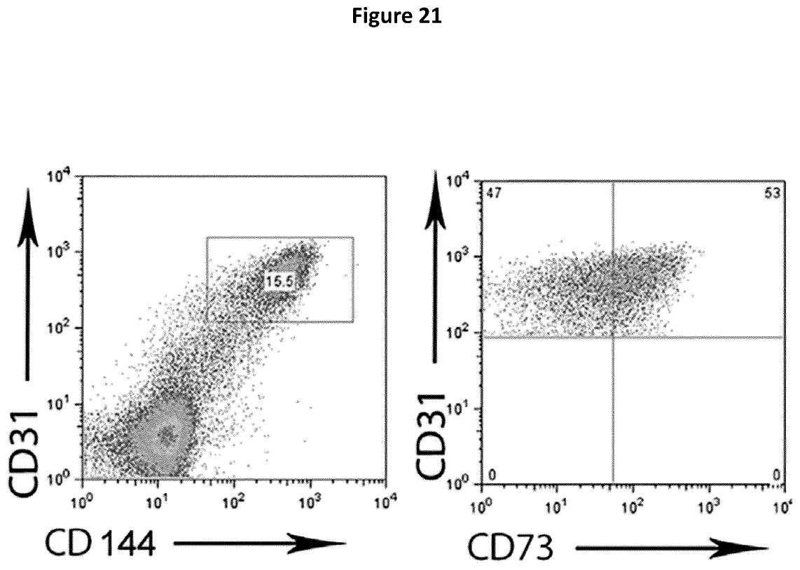

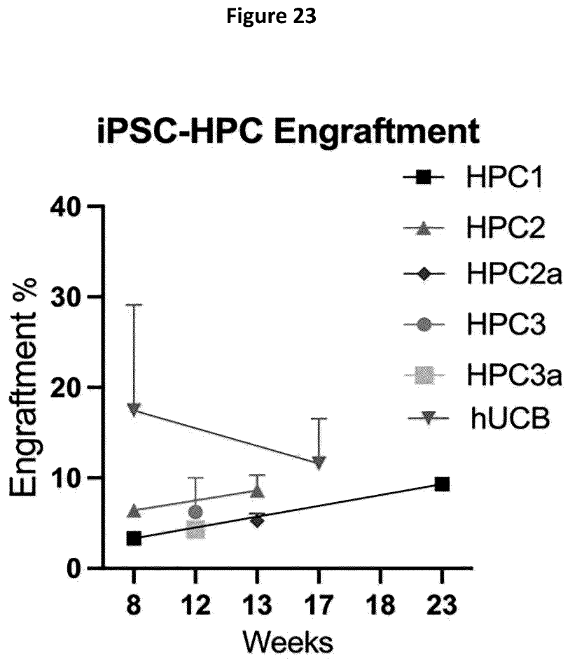

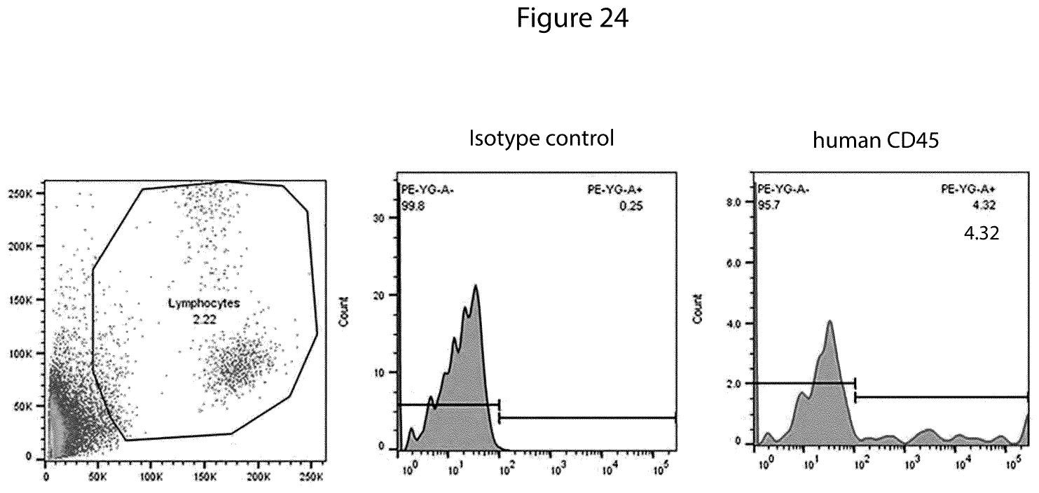

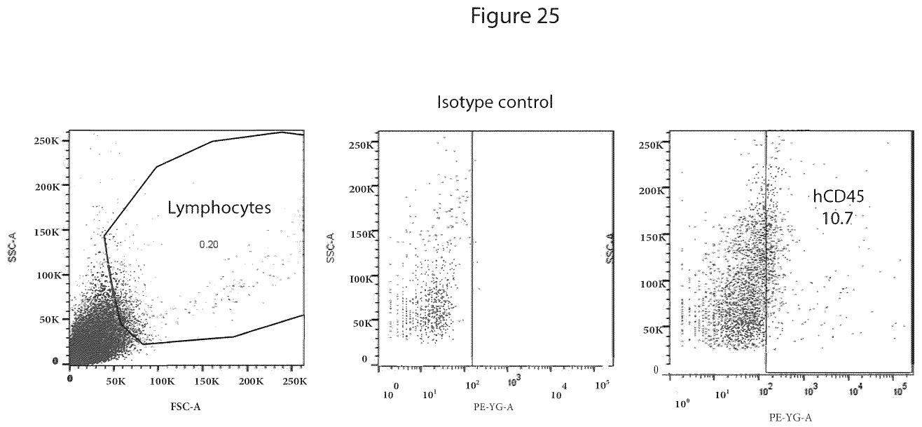

First, a CD34⁺ cell population generated by gently lifting hPSC with enzyme-free solution such as Ca²⁺, Mg²⁺ free PBS and plating on animal free matrix, -recombinant fibronectin then isolating on Day 5 of monolayer differentiation conditions was analyzed (FIG. 21). Specifically, for exemplary monolayer differentiation conditions, pluripotent cells, e.g., hPSCs, were first gently dissociated into single cells using PBS free of Ca²⁺ and Mg²⁺. Pluripotent stem cells were then plated onto fibronectin-coated dishes at a seeding density of 1-5×10⁶ cells per 60 mm dish and cultured overnight to produce colonies of approximately 10-100 cells. Differentiation of cells generated by the above methods was then induced by incubating them for about 2 days in an induction media comprising ascorbic acid (e.g., at a concentration of 60 μg/mL) and a Wnt activator, e.g., CHIR99021 (CAS registry number 252917-06-9; at a concentration of 3-8 μM), followed by removing the Wnt activator and continuing incubation for about 3 days to produce hemogenic endothelium. A subset of the isolated CD34+ cell population cells exhibited an engraftable CD90⁺/CD73⁻ signature (FIG. 17). When injected into NSG mice, these cells (hematopoietic progenitor cell time point 1, HPC1) generated up to 5.8% of human CD45+ cells by 8 weeks, and continued to expand to 9% at 23 weeks post injection demonstrating long term engraftment (FIGS. 23-25).

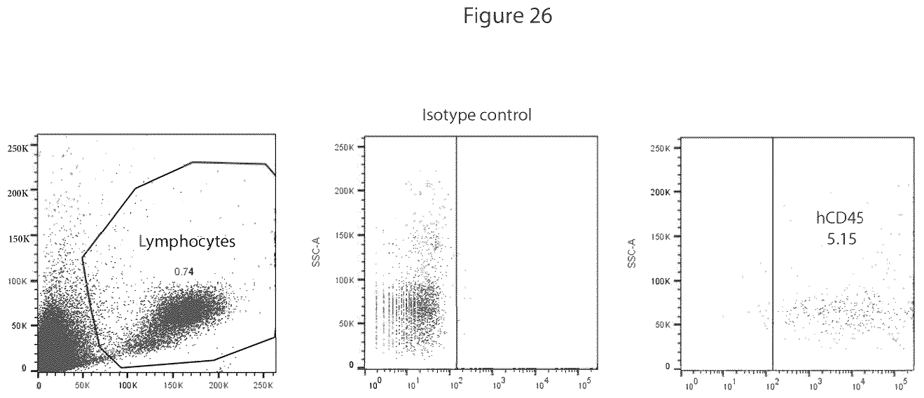

Next, Day 5 (hematopoietic progenitor cell time point 2, HPC2) isolated cells were co-cultured with OP9-DLL4 cells in hematopoietic stem cell cytokines (50 ng/mL each of stem cell factor, SCF; thrombopoietin, TPO; and IL-3) for 3 days. When injected into NSG mice, these cells produced up to 6.4% CD45⁺ cells 8 weeks after the injection and up to 10% CD45⁺ cells 12 weeks after the injection (FIG. 23 and FIG. 26).

### Organoid System for Engraftable Cells

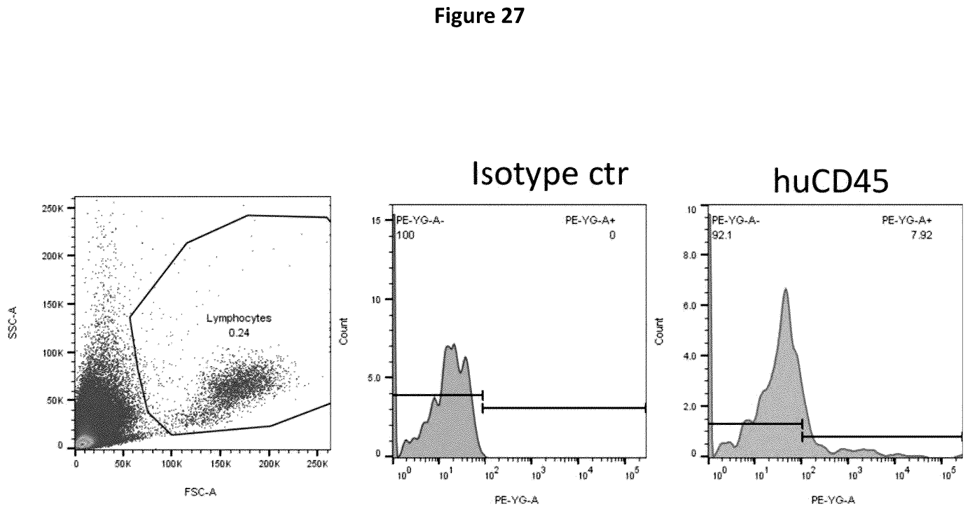

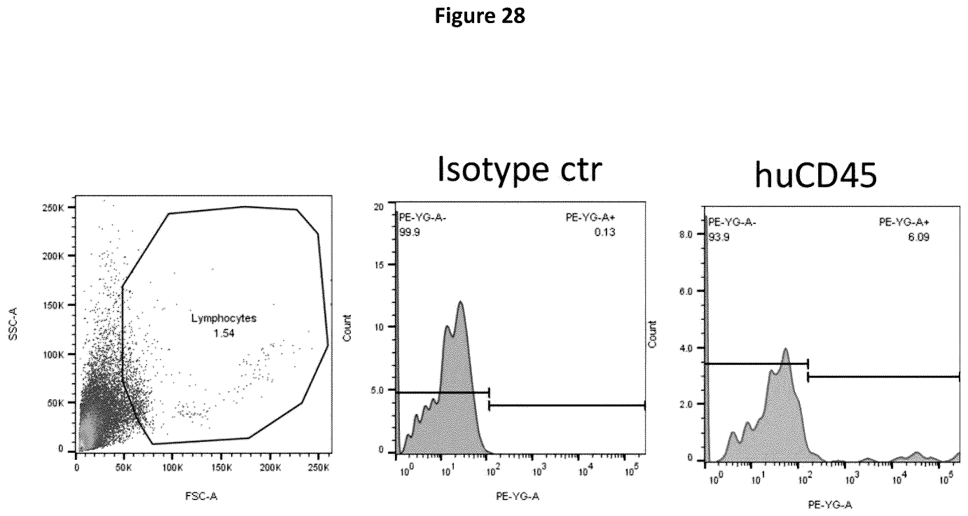

To generate a three-dimensional vessel organoid capable of producing highly functional cells of engraftable progenitor cells, a Day 5 monolayer of hPSCs was gently dispersed into small clusters of approximately 10-100 cells, and carefully plated onto OP9-DLL4-coated plates (or, alternatively, onto plates coated with animal-free matrix, e.g., recombinant fibronectin) with serial addition of cytokines (50 ng/mL each of stem cell factor, SCF; thrombopoietin, TPO; and IL-3) over the course of 72 hours, until the initiation of the formation of the vessel-like structures was achieved. Culturing continued in differentiation media until cells appeared within the vessels. A separate experiment conducted using a VEC-tdTomato/CD43-eGFP cell line (obtained from Dr. Igor Slukvin) demonstrated that these cells expressed CD43⁺ (FIG. 2 and FIG. 3). When the floating cells emerged from the vascular organoids, they were collected and approximately 1×10⁶ to 2×10⁶ cells injected intravenously (i.v.) via tail vein injection into irradiated NSG mice. Flow cytometry analysis demonstrated an average of 5% CD45⁺ cell engraftment at approximately week 12 (FIG. 23 and FIGS. 27-28).

Interestingly, while cord blood isolated CD34⁺ cells showed the highest engraftment at 8 weeks, the number of CD45⁺ continuously declined, while the percentage of CD45⁺ cells generated from iPSC-derived CD34⁺ cells continued to increase (FIG. 23 and FIG. 29).

The present example demonstrated the engraftment of HPC (HSC) cells at several stages of differentiation, including cells conditioned on OP9-DLL4 cells and within vascular organoid, as well as CD34⁺ progenitor cells, which do not yet express hematopoietic markers, e.g., CD43 or CD45. Based on the assessment of differentiation markers, i.e., when cells express VE-cadherin, CD31, and CD34, while lacking the expression of CD43, CD73 and CD235a, this stage of development may be regarded as hemogenic endothelium (HE). The engraftment of HE indicates that further maturation of these cells occurred in a host stromal microenvironment. Confirming previous findings disclosed herein, the present differentiation system produces efficient hematopoiesis in adherent cultures, which allows for avoidance of stromal cells and limits use of growth factors. Engraftment at this early stage is likely in part due to the lack of exposure of the progenitor cells to cytokines, which may cause premature commitment of the progenitors.

The present work provides strong evidence for long-term engraftment of blood progenitors developed from hPSCs. In particular, findings described in the present example show that cells at an earlier stage of hPSC differentiation, e.g., HE stage, in accordance with differentiation approaches disclosed herein, are engraftable [45]. The development of engraftable cells is an important result since such cells can be used in many therapeutic applications. Production of such cells at an earlier stage simplifies the procedure of obtaining engraftable cells as compared to other strategies requiring further differentiation steps. Contribution of blood lineages particularly for the contribution of myeloid and lymphoid cells, may be analyzed utilizing markers for myeloid cells such as CD11b/14/15/16/33; and markers for lymphoid cells, for example T cells markers CD3, CD4, CD8; B cell marker CD19, and NK cell marker CD56.

### Example 10. Induced Pluripotent Stem Cell (iPSC)-Derived Endothelium to Engineer Biologically Active Blood Vessels

The present example sets forth proof-of-concept data in support of a tissue engineering strategy using induced pluripotent stem cell (iPSC)-derived endothelium of the present disclosure to engineer biologically active blood vessels for use in, e.g., the next generation of patient-specific, tissue-engineered vessels to build artificial organs, and as a replacement for current autologous or synthetic grafts.

Endothelial progenitor cells (EPCs) are highly sought for cell-based therapies, particularly for their potential to repair ischemic tissues and fabricate blood vessels but their sources are very limited. Induced pluripotent stem cells (iPSCs) provide the potential for patient-specific cells with similar characteristics to human embryonic stem cells (hESCs).

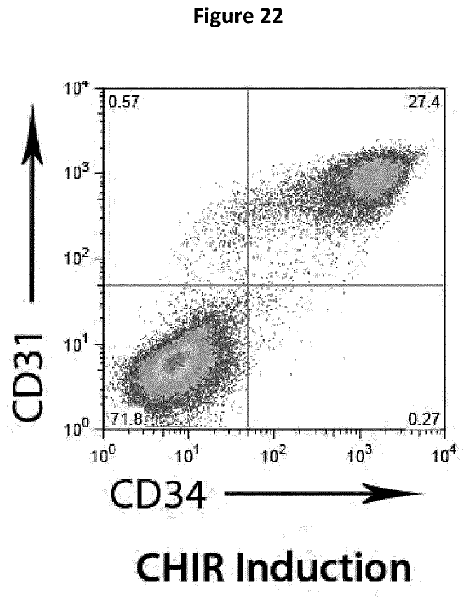

Hematopoietic and endothelial cells (ECs) share a common developmental pathway, yielding development of primitive and definitive hematopoietic progenitors from endothelial cells termed hemogenic endothelium (HE). Hematopoietic differentiation of iPSCs offers unique opportunities for regenerative medicine by generating transplantable ECs and blood cells, and provides important tools for disease modeling and drug discoveries. Indeed, such an approach may be used in various therapeutic applications/methods such as, but not limited to, ischemia treatment, artificial vessels and tissue vascularization. In the present example, an exemplary protocol for in vitro hemato-vascular differentiation of iPSCs as disclosed herein, distinguished both by its robustness and efficiency, was developed and applied. Biological activity of iPSC-derived ECs was confirmed by assessment of proliferation, CD31/CD/34/CD144 markers (FIG. 21 and FIG. 22), stability, and essential functions (von Willebrand factor, vWF, tube forming assay (not shown).

### vWF Assay

Cells were fixed with 3.2% paraformaldehyde for 30 min and permeabilized for 5 min with 0.1% Triton-x-100 in PBS. The cells were then treated with Dako Protein Block for 25 min in order to prevent nonspecific antibody binding. Following this, the cells were incubated with VWF (R&D Systems) (3 hrs). After washing the cells 3× with Dako Washing Buffer (WB), the appropriate Alexa Fluor-conjugated secondary antibodies (Invitrogen) were added to cell culture wells; the incubation time was 45 min. All antibody dilutions were performed according to manufacturers' instructions. Samples were then washed once more with WB and incubated with DAPI (Sigma Aldrich) for 3 minutes. The immunofluorescent cells were visualized with Leica DM IRB inverted microscope system (Leica, Germany) equipped with a digital camera Retiga 4000R (Qlmaging, Canada), which was controlled with Openlab software version 5.0.2 (Perkin-Elmer).

### Tube Forming Assay

Matrigel (Corning) was thawed overnight at 4° C. The following morning, matrix coating was added to 12-well cell culture plates, which were incubated for 30 min at 37° C. and 5% C02. The cells were seeded at a density of 2.75×10⁵ cells per well and incubated for 6 hrs in VascuLife EnGS medium (LifeLine). After the incubation period, the cells were treated with the cell permeable dye Calcein-AM (2 μg/mL) and incubated for 30 min at 37° C. and 5% CO₂. Afterwards, the 12-well cell culture plates were ready for tube network visualization under the Leica DM IRB inverted microscope system (Leica, Germany) equipped with a digital camera Retiga 4000R (Qlmaging, Canada).

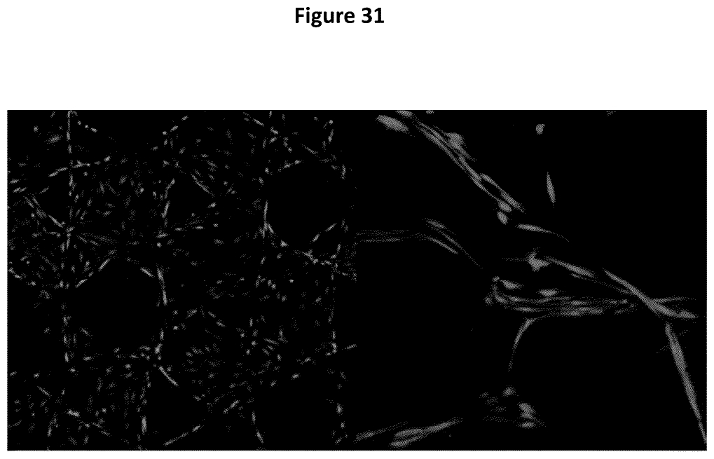

Findings such as these provide support for iPSC-EC progenitor acquisition of functional features of regional endothelium suitable for a wide range of regenerative applications. Feasibility of survival and function of HE upon cellular transplantation were demonstrated by data generated in engraftment experiments (see, e.g., Example 9), as depicted in FIGS. 23-25, and by bioengineering of bioactive small-caliber vascular grafts with three-dimensional (3D) printed blood vessel networks (FIG. 31).

Fibrosis Model To produce fibrosis model, the Scar-in-the-Jar model is adopted (Chen et al., 2009; Stebler and Raghunath, 2021) by using dextran sulphate as macromolecular crowding (MMC) agent, normal lung fibroblasts as tissue-specific cell population and TGFβ1 to induce their myofibroblast trans-differentiation.

Normal lung fibroblasts (CCL-186, ATCC, United States) are cultured with DMEM supplemented with 10% FBS and 1% penicillin/streptomycin; media are changed every 2-3 days. For the fibrosis induction cells are cultured at 25,000 cells/cm² and allowed to attach for 24 h, after which the culture media are changed to media containing 100 μM L-ascorbic acid 2-phosphate sesquimagnesium salt hydrate, 100 μg/ml 500 kDa Dextran Sulphate (DxS), and 5 ng/ml TGFβ1.

To assess antifibrotic activity of (NKG2A) knock-out (KO) iPSC-Natural Killer cells compared to wild type unmodified NKG2A iPSC-NK cells, NK cells will be co-incubated with the fibrotic cells. To evaluate NK cytotoxicity, cell death will be measured by cell viability using violet-red staining combined with propidium iodide (PI)-based viability assays to quantify viable vs necrotic fibroblast cells. Additionally, we will establish the level of NK cell activation by assessing their proliferation. In order to count the NK cells, iPSC NK cells will be stained in using CellTracker Green CMFDA (5-chloromethylfluorescein diacetate) dye.

F-actin, α-SMA, and Collagen I expression in the fibrotic model will be assessed before and after incubation with NK cells.

Properly activated iPSC-NK cells will effectively eliminate fibrosis. Additionally, NKG2A-KO-iPSC-NKs will work more efficiently by defying the inhibitory signaling produced by fibrotic and senescent cells comprising the scar. Addition of the antibodies specific to fibrotic extracellular components (markers) specific to the fibrotic cells may further advance the antifibrotic activity of NK cells.

Chen, C. Z., Peng, Y. X., Wang, Z. B., Fish, P. V., Kaar, J. L., Koepsel, R. R., Russell, A. J., Lareu, R. R., Raghunath, M., 2009. The Scar-in-a-Jar: studying potential antifibrotic compounds from the epigenetic to extracellular level in a single well. British journal of pharmacology 158, 1196-1209.

Stebler, S., Raghunath, M., 2021. The Scar-in-a-Jar: In Vitro Fibrosis Model for Anti-Fibrotic Drug Testing. Methods Mol Biol 2299, 147-156.

### REFERENCES

1. Slukvin I I. Hematopoietic specification from human pluripotent stem cells: current advances and challenges toward de novo generation of hematopoietic stem cells. Blood. 2013; 122:4035-46.
2. Kaufman D S. Toward clinical therapies using hematopoietic cells derived from human pluripotent stem cells. Blood. 2009; 114:3513-23.
3. Vo L T, Daley G Q. De novo generation of HSCs from somatic and pluripotent stem cell sources.

Blood. 2015; 125:2641-8.
4. Batta K, Menegatti S, Garcia-Alegria E, et al. Concise Review: Recent advances in the in vitro derivation of blood cell populations. Stem Cells Transl Med. 2016; 10:1330-7.
5. Chou S T, Byrska-Bishop M, Tober J M, et al. Trisomy 21-associated defects in human primitive hematopoiesis revealed through induced pluripotent stem cells. Proc Natl Acad Sci USA. 2012; 109:17573-8.
6. Maclean G A, Menne T F, Guo G, et al. Altered hematopoiesis in trisomy 21 as revealed through in vitro differentiation of isogenic human pluripotent cells. Proc Natl Acad Sci USA. 2012; 109:17567-72.
7. Lachmann N, Ackermann M, Frenzel E, et al. Large-scale hematopoietic differentiation of human induced pluripotent stem cells provides granulocytes or macrophages for cell replacement therapies. Stem Cell Rep. 2015; 4:282-96.
8. Menon T, Firth A L, Scripture-Adams D D, et al. Lymphoid regeneration from gene-corrected SCID-X1 subject-derived iPSCs. Cell Stem Cell. 2015; 16:367-72.
9. Laskowski T J, Van Caeneghem Y, Pourebrahim R, et al. Gene correction of iPSCs from a Wiskott-Aldrich syndrome patient normalizes the lymphoid developmental and functional defects.

Stem Cell Rep. 2016; 7(2):139-148.
10. Kang H, Minder P, Park M A, et al. CCR5 Disruption in induced pluripotent stem cells using CRISPR/Cas9 provides selective resistance of immune cells to CCR5-tropic HIV-1 Virus. Mol Ther Nucleic Acids. 2015;4:e268.
11. Sturgeon C M, Ditadi A, Awong G, et al. Wnt signaling controls the specification of definitive and primitive hematopoiesis from human pluripotent stem cells. Nat Biotechnol. 2014; 32:554-61.
12. Themeli M, Kloss C C, Ciriello G, et al. Generation of tumor-targeted human T lymphocytes from induced pluripotent stem cells for cancer therapy. Nat Biotechnol. 2013; 31:928-33.
13. Kennedy M, Awong G, Sturgeon C M, et al. T lymphocyte potential marks the emergence of definitive hematopoietic progenitors in human pluripotent stem cell differentiation cultures. Cell Rep. 2012; 2:1722-35.
14. Timmermans F, Velghe I, Vanwalleghem L, et al. Generation of T cells from human embryonic stem cell-derived hematopoietic zones. J Immunol. 2009; 182:6879-88.
15. Smith M J, Webber B R, Mohtashami M, et al. Concise Review: In vitro T-cell generation from adult, embryonic, and induced pluripotent stem cells: many roads to one destination. Stem Cells. 2015; 33(11):3174-3180.
16. Knorr D A, Ni Z, Hermanson D, et al. Clinical-scale derivation of natural killer cells from human pluripotent stem cells for cancer therapy. Stem Cells Transl Med. 2013; 2:274-83.
17. Kitayama S, Zhang R, Liu T Y, et al. Cellular adjuvant properties, direct cytotoxicity of re-differentiated Valpha24 invariant NKT-like cells from human induced pluripotent stem cells. Stem Cell Rep. 2016; 6:213-27.
18. Carpenter L, Malladi R, Yang C T, et al. Human induced pluripotent stem cells are capable of B-cell lymphopoiesis. Blood. 2011; 117:4008-11.
19. Slukvin I I. Generating human hematopoietic stem cells in vitro-exploring endothelial to hematopoietic transition as a portal for stemness acquisition. FEBS Lett. 2016; 590(22):4126-4143.
20. Vodyanik M A, Bork J A, Thomson J A, et al. Human embryonic stem cellderived CD34⁺ cells: efficient production in the coculture with OP9 stromal cells and analysis of lymphohematopoietic potential. Blood. 2005; 105:617-26.
21. Elcheva I, Brok-Volchanskaya V, Kumar A, et al. Direct induction of haematoendothelial programs in human pluripotent stem cells by transcriptional regulators. Nat Commun. 2014; 5:4372.
22. Doulatov S, Vo L T, Chou S S, et al. Induction of multipotential hematopoietic progenitors from human pluripotent stem cells via respecification of lineage-restricted precursors. Cell Stem Cell. 2013; 13:459-70.
23. Nakajima-Takagi Y, Osawa M, Oshima M, et al. Role of SOX17 in hematopoietic development from human embryonic stem cells. Blood. 2013; 121:447-58.
24. Sturgeon C M, Ditadi A, Clarke R L, et al. Defining the path to hematopoietic stem cells. Nat Biotechnol. 2013; 31:416-8.
25. Choi K D, Vodyanik M, Slukvin I I. Hematopoietic differentiation and production of mature myeloid cells from human pluripotent stem cells. Nat Protoc. 2011; 6:296-313.
26. Murry C E, Keller G. Differentiation of embryonic stem cells to clinically relevant populations: lessons from embryonic development. Cell. 2008; 132:661-80.
27. Song P, An J and Zou M. Immune Clearance of Senescent Cells to Combat Ageing and Chronic Diseases. Cells 2020, 9(3), 671.
28. Malavolta M, Giacconi R, Brunetti D, Provinciali M, Maggi F. Exploring the Relevance of Senotherapeutics for the Current SARS-CoV-2 Emergency and Similar Future Global Health Threats. Cells. 2020 Apr. 8; 9(4):909.
29. Marin R, Ruiz-Cabello F, Pedrinaci S, Mendez R, Jimenez P, Geraghty D R, Garrido F. Analysis of HLA-E expression in human tumors. Immunogenetics. 2003 February;54(11):767-75.
30. Pereira et al., Senescent cells evade immune clearance via HLA-E-mediated NK and CD8⁺ T cell inhibition. Nature Communications volume 10, Article number: 2387 (2019).
31. M P Coghlan et al., Selective small molecule inhibitors of glycogen synthase kinase-3 modulate glycogen metabolism and gene transcription. Chem Biol. 2000 October;7(10):793-803.
32. N Sato et al., Maintenance of pluripotency in human and mouse embryonic stem cells through activation of Wnt signaling by a pharmacological GSK-3-specific inhibitor. Nat Med. 2004 January;10(1):55-63.
33. J M Atkinson et al., Activating the Wnt/β-Catenin Pathway for the Treatment of Melanoma-Application of LY2090314, a Novel Selective Inhibitor of Glycogen Synthase Kinase-3. PLoS One. 2015 Apr. 27; 10(4):e0125028. doi: 10.1371/journal.pone.0125028. eCollection 2015.
34. PVN Bodine et al., A small molecule inhibitor of the Wnt antagonist secreted frizzled-related protein-1 stimulates bone formation. Bone. 2009 June;44(6):1063-8.
35. R M Suciu et al., Selective Irreversible Inhibitors of the Wnt-Deacylating Enzyme NOTUM Developed by Activity-Based Protein Profiling. ACS Med Chem Lett. 2018 May 26;9(6):563-568.
36. A M Gilbert et al., Hit to lead studies on (hetero)arylpyrimidines—agonists of the canonical Wnt-beta-catenin cellular messaging system. Bioorg Med Chem Lett. 2010 Jan. 1; 20(1):366-70.
37. T Miyabayashi et al., Wnt/beta-catenin/CBP signaling maintains long-term murine embryonic stem cell pluripotency. Proc Natl Acad Sci USA. 2007 Mar. 27; 104(13):5668-73.
38. Q Zhang et al., Small-molecule synergist of the Wnt/beta-catenin signaling pathway. Proc Natl Acad Sci USA. 2007 May 1;104(18):7444-8.
39. R Pai et al., Deoxycholic acid activates beta-catenin signaling pathway and increases colon cell cancer growth and invasiveness. Mol Biol Cell. 2004 May; 15(5):2156-63.
40. J Lui et al., A small-molecule agonist of the Wnt signaling pathway. Angew Chem Int Ed Engl. 2005 Mar. 18; 44(13):1987-90.
41. Goya R G, Lehmann M, Chiavellini P, Canatelli-Mallat M, Herenu C B, Brown O A.

Rejuvenation by cell reprogramming: a new horizon in gerontology. Stem Cell Res Ther. 2018;9(1):349.
42. Galat V, Malchenko S, Galat Y, Ishkin A, Nikolsky Y, Kosak S T, et al. A model of early human embryonic stem cell differentiation reveals inter- and intracellular changes on transition to squamous epithelium. Stem Cells Dev. 2012; 21(8):1250-63.
43. Lin T, Ambasudhan R, Yuan X, Li W, Hilcove S, Abujarour R, et al. A chemical platform for improved induction of human iPSCs. Nat Methods. 2009; 6(11):805-8.
44. Galat V, Galat Y, Perepitchka M, Jennings L J, Iannaccone P M, Hendrix M J. Transgene Reactivation in Induced Pluripotent Stem Cell Derivatives and Reversion to Pluripotency of Induced Pluripotent Stem Cell-Derived Mesenchymal Stem Cells. Stem Cells Dev. 2016; 25(14):1060-72.
45. Galat Y, Dambaeva S, Elcheva I, Khanolkar A, Beaman K, Iannaccone P M, et al. Cytokine-free directed differentiation of human pluripotent stem cells efficiently produces hemogenic endothelium with lymphoid potential. Stem Cell Res Ther. 2017;8(1):67.
46. Galat Y, Perepitchka M, Elcheva I, Iannaccone S, Iannaccone P M, Galat V. iPSC-derived progenitor stromal cells provide new insights into aberrant musculoskeletal development and resistance to cancer in down syndrome. Sci Rep. 2020;10(1):13252.
47. Kumar A, Lee J H, Suknuntha K, D'Souza S S, Thakur A S, Slukvin I I. NOTCH Activation at the Hematovascular Mesoderm Stage Facilitates Efficient Generation of T Cells with High Proliferation Potential from Human Pluripotent Stem Cells. J Immunol. 2019 Feb. 1; 202(3):770-776.
48. Denman C J, Senyukov V V, Somanchi S S, et al. Membrane-bound IL-21 promotes sustained ex vivo proliferation of human natural killer cells. PLoS One. 2012;7(1):e30264.
49. Liu S, Galat V, Galat Y, Lee YKA, Wainwright D, Wu J. N K cell-based cancer immunotherapy: from basic biology to clinical development. J Hematol Oncol. 2021;14(1):7.
50. Oyer J L, Igarashi R Y, Kulikowski A R, Colosimo D A, Solh M M, Zakari A, Khaled Y A, Altomare D A, Copik A J. Generation of highly cytotoxic natural killer cells for treatment of acute myelogenous leukemia using a feeder-free, particle-based approach. Biol Blood Marrow Transplant. 2015 April; 21(4):632-9.
51. Traver D Going with the flow: how shear stress signals the emergence of adult hematopoiesis. J Exp Med. 2015;212(5):600. doi:10.1084/jem.2125insight4
52. Abed S et al., Transplantation of Macaca cynomolgus iPS-derived hematopoietic cells in NSG immunodeficient mice. Haematologica. 2015 October; 100(10): e428-e431. doi: 10.3324/haematol.2015.127373
53. Vanhee, S et al., In vitro human embryonic stem cell hematopoiesis mimics MYB-independent yolk sac hematopoiesis. Haematologica. 2015 February; 100(2): 157-166. doi: 10.3324/haematol.2014.112144
54. Vanuytsel K et al. Multi-Modal Profiling of Human Fetal Liver-Derived Hematopoietic Stem Cells Reveals the Molecular Signature of Engraftment Potential. Posted Nov. 12, 2020. bioRxiv doi: 10.1101/2020.11.11.378620
55. Sugimura R et al., Haematopoietic stem and progenitor cells from human pluripotent stem cells. Nature. 2017 May 25;545(7655):432-438. doi: 10.1038/nature22370. Epub 2017 May 17.
56. Abe T et al., Fetal sheep support the development of hematopoietic cells in vivo from human induced pluripotent stem cells. Experimental Hematology Volume 95, March 2021, Pages 46-57.e8.doi: 10.1016/j.exphem.2020.12.006
57. Narayanan D L & Phadke S R. Concepts, Utility and Limitations of Cord Blood Banking: What Clinicians Need to Know. Indian J Pediatr. 2019 January;86(1):44-48. doi: 10.1007/s12098-018-2651-y. Epub 2018 Mar. 20.
58. Galat Y et al., Application of small molecule CHIR99021 leads to the loss of hemangioblast progenitor and increased hematopoiesis of human pluripotent stem cells. Experimental Hematology Volume 65, September 2018, Pages 38-48.e1. doi: 10.1016/j.exphem.2018.05.007.
59. Narayanan, D L and Phadke S R. Concepts, Utility and Limitations of Cord Blood Banking: What Clinicians Need to Know. Indian J Pediatr. 2019 January;86(1):44-48. doi: 10.1007/s12098-018-2651-y.
60. Brok-Volchanskaya, V. S., D. A. Bennin, K. Suknuntha, L. C. Klemm, A. Huttenlocher and I. Slukvin (2019). “Effective and Rapid Generation of Functional Neutrophils from Induced Pluripotent Stem Cells Using ETV2-Modified mRNA.” Stem Cell Reports 13(6): 1099-1110.
61. Cao, X., G. K. Yakala, F. E. van den Hil, A. Cochrane, C. L. Mummery and V. V. Orlova (2019). “Differentiation and Functional Comparison of Monocytes and Macrophages from hiPSCs with Peripheral Blood Derivatives.” Stem Cell Reports 12(6): 1282-1297.
62. Iriguchi, S., Y. Yasui, Y. Kawai, S. Arima, M. Kunitomo, T. Sato, T. Ueda, A. Minagawa, Y. Mishima, N. Yanagawa, Y. Baba, Y. Miyake, K. Nakayama, M. Takiguchi, T. Shinohara, T. Nakatsura, M. Yasukawa, Y. Kassai, A. Hayashi and S. Kaneko (2021). “A clinically applicable and scalable method to regenerate T-cells from iPSCs for off-the-shelf T-cell immunotherapy.” Nat Commun 12(1): 430.
63. Kammers, K., M. A. Taub, B. Rodriguez, L. R. Yanek, I. Ruczinski, J. Martin, K. Kanchan, A. Battle, L. Cheng, Z. Z. Wang, A. D. Johnson, J. T. Leek, N. Faraday, L. C. Becker and R. A. Mathias (2021). “Transcriptional profile of platelets and iPSC-derived megakaryocytes from whole-genome and RNA sequencing.” Blood 137(7): 959-968.
64. Mesquitta, W. T., M. Wandsnider, H. Kang, J. Thomson, O. Moskvin, K. Suknuntha and Slukvin, I I (2019). “UM171 expands distinct types of myeloid and NK progenitors from human pluripotent stem cells.” Sci Rep 9(1): 6622.
65. Lyman, G. H. and Poniewierski M. S. A Patient Risk Model of Chemotherapy-Induced Febrile Neutropenia: Lessons Learned From the ANC Study Group. J Natl Compr Canc Netw. 2017 December;15(12):1543-1550.
66. Khaddour K., Hana C. K., and Mewawalla P. Hematopoietic Stem Cell Transplantation, StatPearls, Treasure Island (FL), 2021.
67. Majumder A., Suknuntha K., Bennin D., Klemm L., Brok-Volchanskaya V. S., Huttenlocher A., and Slukvin I. Generation of Human Neutrophils from Induced Pluripotent Stem Cells in Chemically Defined Conditions Using ETV2 Modified mRNA. Star Protocols. Volume 1, Issue 2, 18 Sep. 2020, 100075

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

SEQUENCE LISTING
guide RNA encoding sequence
SEQ ID NO: 1
TGAAGGTTTAATTCCGCATGTTTTAGAGCTAGAAA
TAGCAAGTTAAAATAAGGCTAGTCCGTTATCAACT
TGAAAAAGTGGCACCGAGTCGGTGC
guide RNA encoding sequence
SEQ ID NO: 2
CGTTGCTGCCTCTTTGGGTTGTTTTAGAGCTAGAA
ATAGCAAGTTAAAATAAGGCTAGTCCGTTATCAAC
TTGAAAAAGTGGCACCGAGTCGGTGC
guide RNA encoding sequence
SEQ ID NO: 3
TGAAGGTTTAATTCCGCATAGG
guide RNA encoding sequence
SEQ ID NO: 4
CGTTGCTGCCTCTTTGGGTTTGG
oligonucleotide
SEQ ID NO: 5
UUAAUUCCGCAUAGGUUAUUUCdTdT
oligonucleotide
SEQ ID NO: 6
GAAAUAACCUAUGCGGAAUUAAdTdT
oligonucleotide
SEQ ID NO: 7
AAGAUAAGACAGAUAAUUCCCdTdT
oligonucleotide
SEQ ID NO: 8
GGGAAUUAUCUGUCUUAUCUUdTdT
target
SEQ ID NO: 9
GAAATAACCTATGCGGAATTAA
target
SEQ ID NO: 10
GGGAATTATCTGTCTTATCTT

1. A method for producing natural killer (NK) cells from pluripotent stem cells, the method comprising:
a) plating the pluripotent stem cells at a seeding density of 1-5×10⁶ cells per 60 mm dish and culturing them overnight to produce colonies of 10-100 cells,
b) inducing differentiation of the cells generated in step (a) by incubating said cells for about 2 days in an induction media comprising ascorbic acid and a Wnt activator,
c) removing the Wnt activator and continuing incubation for about 3 days,
d) detaching cell layers formed in step (c) enzymatically and/or mechanically, and plating them onto matrix-coated dishes or onto a monolayer of feeder cells,
e) culturing the cells plated in step (d) for about 12 days in alpha-MEM differentiation media comprising 10% FBS, ascorbic acid, SCF, IL-3, IL-7, IL-15, and FLT3-L, resulting in the formation of a three-dimensional vascular organoid which promotes differentiation of NK cells,
f) collecting floating cells formed during step (e) and re-plating them onto matrix-coated dishes or onto a semi-confluent monolayer of feeder cells,
g) culturing the replated cells of step (f) for about 10-16 days in maturation media comprising cytokines and the Wnt activator, but not comprising IL-3, to promote NK cell maturation, and
h) optionally, purifying NK cells generated in step (g).

2. The method of claim 1, wherein the purification of NK cells is achieved by isolating CD56+ cells.

3. The method of claim 2, wherein the cytokines present in the maturation media of step (g) comprise SCF, IL-7, and/or IL-15.

4. The method of claim 3, wherein the Wnt activator is selected from Wnt4 protein, CHIR99021 (CAS registry number 252917-06-9), SB-216763, BIO (6-bromoindirubin-3′-oxime), LY2090314, WAY-316606, ABC99, (hetero)arylpyrimidines, IQ1, QS11, Deoxycholic acid (DCA) and 2-amino-4-[3,4-(methylenedioxy)benzyl-amino]-6-(3-methoxyphenyl)pyrimidine.

5. The method of claim 4, wherein the feeder cells are stromal cells.

6. The methods of claim 5, where no cytokines are present in steps (a)-(c).

7. The method of claim 6 wherein the pluripotent stem cells are induced pluripotent stem cells (iPSCs) or embryonic stem cells (ESCs).

8. The method of claim 2, further comprising expanding the NK cells produced in step (g) by culturing them in expansion media comprising IL-2.

9. The method of claim 2, further comprising expanding NK cells produced in step (g) by culturing them in the presence of allogeneic feeder cells; and wherein step (d) further comprises purifying CD31/CD34/CD144 triple-positive cells.

10. The method of claim 1, further comprising treating a disease or disorder in a subject in need thereof comprising administering to the subject a therapeutically effective amount of pluripotent stem cell derived NK.

11. A method of suppressing expression of NKG2A gene in a natural killer (NK) cell, said method comprising subjecting said NK cell or a pluripotent stem cell used to generate said NK cell to a CRISPR-Cas editing system and a guide RNA (gRNA) comprising the sequence selected from SEQ ID NO: 1-4 to generate a NKG2A modified pluripotent stem cell derived NK cell; or administering to said NK cell or expressing in said NK cell a siRNA comprising the sequence selected from SEQ ID NO: 5-10 to generate a NKG2A modified pluripotent stem cell derived NK cell.

12. The method of claim 11, further comprising treating a disease or disorder in a subject in need thereof comprising administering to the subject a therapeutically effective amount of the NKG2A modified pluripotent stem cell derived NK cell or a pharmaceutical composition of the NKG2A modified pluripotent stem cell derived NK cell.

13. The method of claim 12, wherein the disease or the disorder is an infection, a cancer, an autoimmune disease, myocardial infarction, ischemia, liver cirrhosis, lung fibrosis, or liver fibrosis.

14. The method of claim 13, wherein the cancer is a type of solid cancer, type of brain cancer, type of lymphoma or a type of leukemia.

15. The method of claim 13, wherein the infection is a bacterial infection or a viral infection.

16. The method of claim 11, further comprising inducing elimination of senescent cells or cancer cells or virus-infected cells in a subject in need thereof by administering to the subject a therapeutically effective amount of the NKG2A modified pluripotent stem cell derived NK cell or the pharmaceutical composition of the NKG2A modified pluripotent stem cell derived NK cell.

17. The method of claim 16, further comprising treating cancer, or an autoimmune disease, or neutropenia or non-malignant blood disorder in a subject in need thereof comprising administering to the subject a therapeutically effective amount of the HPCs of claims 18-20 or a therapeutically effective amount of the myeloid cells produced by claim 25.

18. A method for producing a population of hematopoietic progenitor cells from pluripotent stem cells, the method comprising:
a) plating the pluripotent stem cells at a seeding density of 1-5×10⁶ cells per 60 mm dish and culturing them overnight to produce colonies of 10-100 cells,
b) inducing differentiation of the cells generated in step (a) by incubating said cells for about 2 days in an induction media comprising ascorbic acid and a Wnt activator,
c) removing the Wnt activator and continuing incubation for about 4-7 days to produce a first population of hematopoietic progenitor cells, and
d) optionally, purifying the first population of hematopoietic progenitor cells generated in step (c).

19. The method of claim 18, further comprising:
e) detaching cell layers formed in step (c) enzymatically and/or mechanically, and plating them onto matrix-coated dishes or onto a monolayer of feeder cells,
f) culturing the cells plated in step (e) for about 2-5 days in alpha-MEM differentiation media comprising 10% FBS, ascorbic acid, and one or more cytokines, resulting in the formation of a second population of hematopoietic progenitor cells, and
g) optionally, purifying the second population of hematopoietic progenitor cells generated in step (f).

20. The method of claim 18, further comprising:
e) detaching cell layers formed in step (c) enzymatically and/or mechanically, and plating them onto matrix-coated dishes or onto a monolayer of feeder cells,
f) culturing the cells plated in step (e) for about 9-13 days in alpha-MEM differentiation media comprising 10% FBS, ascorbic acid, and one or more cytokines, resulting in the formation of a third population of hematopoietic progenitor cells from a three-dimensional vascular organoid, and
g) optionally, purifying the third population of hematopoietic progenitor cells generated in step (f).

21. The method of claim 18, wherein the purifying step is achieved by isolating CD34+ cells.

22. The method of claim 21, wherein step (a) further comprises dissociating the pluripotent cells into single cells using a Ca²⁺ and Mg²⁺ free phosphate-buffered saline (PBS) solution before the plating step.

23. The method of claim 22, wherein the pluripotent stem cells are plated onto a plate that is coated with fibronectin.

24. The method of claim 23, wherein the one or more cytokines used in step (f) comprise SCF, IL-3, and TPO.

25. A method for producing myeloid cells from pluripotent stem cells, the method comprising:
a) plating the pluripotent stem cells at a seeding density of 1-5×10⁶ cells per 60 mm dish and culturing them overnight to produce colonies of 10-100 cells,
b) inducing differentiation of the cells generated in step (a) by incubating said cells for about 2 days in an induction media comprising ascorbic acid and a Wnt activator,
c) removing the Wnt activator and continuing incubation for about 3 days,
d) detaching cell layers formed in step (c) enzymatically and/or mechanically, and plating them onto matrix-coated dishes or onto a monolayer of feeder cells,
e) culturing the cells plated in step (d) for about 12 days in alpha-MEM differentiation media comprising 10% FBS, ascorbic acid, and cytokines specific to a particular myeloid lineage, resulting in the formation of a three-dimensional vascular organoid which promotes differentiation of myeloid cells,
f) collecting floating cells formed during step (e) and re-plating them onto matrix-coated dishes or onto a semi-confluent monolayer of feeder cells,
g) culturing the replated cells of step (f) for about 7-14 days in maturation media comprising cytokines to promote myeloid cell maturation, and
h) optionally, purifying the myeloid cells generated in step (g).

26. The method of claim 25, wherein the purification of myeloid cells is achieved by isolating CD15+ or CD14+ or CD11b+ or CD33+ or CD235+ cells.

27. The method of claim 25, wherein the myeloid cells are selected from granulocytes, monocytes, macrophages, erythrocytes, megakaryocytes, and mast cells.

28. The method of claim 25, further comprising creating artificial vasculature, 3D organs and organoids, or treating myocardial infarction and wound healing in a subject in need thereof comprising administering to the subject a therapeutically effective amount of the HPCs of claims 18-20.
